SEQUENCE LISTING (57 sequences)

SEQ ID NO: 1 - DNA, 2032 bp, Homo sapiens
gggctcggccggggagcgtgcggagtctgcacgccgcggccgtcggtccaacgcggggaggctcagggattttcggcagcagcgacttcatccaccccggaccgccggctggaggacgtgcctgcagccggcgggtacctgcggccaattcggtggaacgaggacgcggactgcgaaggcaggtaccctcctctgaaatcaaaactctttaattaaagagacatgaaatgacaagattttaaaaaaatcccatgaggagaaggcattctaatttaggaagaactcccccttggaaaaaccatcagtgccggaagatttcctattgtgttgatccatggcaaaggagactgcagatacacaagggatattatggagcccagacgacctgaataaaacccttccctactacaaggacagctgtcccttccctacacactccctacaggctgatgagagaccttttttggaagcagaaacttatactttatgctgccttcttcctgactgccaggattatactcttcctttccatcccagatctagcaatgctgttgatgaggctaagtcatgatgatttctttaatatcttggaacacagtagatgcctgatatttgcggatggactggagaaaaactgaaagtataaaccacaacatctcaagagatgtcatgaatggagaagcatatggtaaaatataatgaaaattaaatctactttacaagtggtatcttcttgacaatagtggcattacctgagccagtatcagggaccagaaatacaacataaaactgatacatttctcttaatccccacatgattttgtttttgagtttatagttatcagtgatcattgattaataattgatcagtgatcagtgacaaatgtatactttttctacttgtattgttagccctcagattgatggtagttagcccaggaatggaaagaccaaggggagatgatttaattctagtcaaatattaaaaggtcatcatgtaaatgaaataatcccaagactacaactagaggggcagatttcagcttactattagggagtaatttcaacaggatatggaagggatatccaaatggcagggaggccattgtgggatatgacagggacgccattgcaggaaatataagaacagaagatcctctgggggatttttgttctttgtgggagtttggattagctgctcctgcctgggatcctttcagctctcaattctgtgaaactcaacaatgaaagcagaggtgttttcctttacttagctagtgccaacacaaaactttgaactcaagtgacctgataacataactgtgggtcaataccgaggaactaacactcctagttccttttcaattccccaagacaacatcagtatagattttcaagatgaatatatgcccagtaagtatggcttatcctgtactggaagaataggttctttcacacttaatgtgattattttttgtcttcatagggtgacttcaagtcttttttaatgtgaaccttacagtggggtttgttctgaatatatttatttgcatttgatatagagaggctcaaaaaaggaaaactgacggacctaataaaataaaaaaatagtagttgaataaaaaaatttctggagggacacacaaataatggttactctctctgcatgccatgggagatagtggtgaagggaggagggacacttttgcaattcatttgatctcttccacagtatctgaattatttgcaagtatacgtattccatttgtaataaaaaagtaaagactagaaattctttttggaaaactgtacatactgtagaacatggctctgtgttgtttgtctctggatttccagtgactatcaaataggtgctcaagaaattggataattatgtgaatgaattcatgaattgaatgaatgaatgaacagactatgctgttctgtttgaaaataagtatggacttacaagaatgaaaagtcttcaacacttggaatatgttttcaataaagatcaggcctctcatctata

SEQ ID NO: 2 - DNA, 2184 bp, Homo sapiens
aagcccaagaggctttccctaactccaaaacggcgacctttaaagtaagggagggttggggcaccacagcggccggatttctaattgtggaaagtttaaacaaatttgtccctcggggaatgggggaagggagagaagaacaagtctgggctcggccggggagcgtgcggagtctgcacgccgcggccgtcggtccaacgcggggaggctcagggattttcggcagcagcgacttcatccaccccggaccgccggctggaggacgtgcctgcagccggcgggtacctgcggccaattcggtggaacgaggacgcggactgcgaaggcaggtaccctcctctgaaatcaaaactctttaattaaagagacatgaaatgacaagattttaaaaaaatcccatgaggagaaggcattctaatttaggaagaactcccccttggaaaaaccatcagtgccggaagatttcctattgtgttgatccatggcaaaggagactgcagatacacaagggatattatggagcccagacgacctgaataaaacccttccctactacaaggacagctgtcccttccctacacactccctacaggctgatgagagaccttttttggaagcagaaacttatactttatgctgccttcttcctgactgccaggattatactcttcctttccatcccagatctagcaatgctgttgatgaggctaagtcatgatgatttctttaatatcttggaacacagtagatgcctgatatttgcggatggactggagaaaaactgaaagtataaaccacaacatctcaagagatgtcatgaatggagaagcatatggtaaaatataatgaaaattaaatctactttacaagtggtatcttcttgacaatagtggcattacctgagccagtatcagggaccagaaatacaacataaaactgatacatttctcttaatccccacatgattttgtttttgagtttatagttatcagtgatcattgattaataattgatcagtgatcagtgacaaatgtatactttttctacttgtattgttagccctcagattgatggtagttagcccaggaatggaaagaccaaggggagatgatttaattctagtcaaatattaaaaggtcatcatgtaaatgaaataatcccaagactacaactagaggggcagatttcagcttactattagggagtaatttcaacaggatatggaagggatatccaaatggcagggaggccattgtgggatatgacagggacgccattgcaggaaatataagaacagaagatcctctgggggatttttgttctttgtgggagtttggattagctgctcctgcctgggatcctttcagctctcaattctgtgaaactcaacaatgaaagcagaggtgttttcctttacttagctagtgccaacacaaaactttgaactcaagtgacctgataacataactgtgggtcaataccgaggaactaacactcctagttccttttcaattccccaagacaacatcagtatagattttcaagatgaatatatgcccagtaagtatggcttatcctgtactggaagaataggttctttcacacttaatgtgattattttttgtcttcatagggtgacttcaagtcttttttaatgtgaaccttacagtggggtttgttctgaatatatttatttgcatttgatatagagaggctcaaaaaaggaaaactgacggacctaataaaataaaaaaatagtagttgaataaaaaaatttctggagggacacacaaataatggttactctctctgcatgccatgggagatagtggtgaagggaggagggacacttttgcaattcatttgatctcttccacagtatctgaattatttgcaagtatacgtattccatttgtaataaaaaagtaaagactagaaattctttttggaaaactgtacatactgtagaacatggctctgtgttgtttgtctctggatttccagtgactatcaaataggtgctcaagaaattggataattatgtgaatgaattcatgaattgaatgaatgaatgaacagactatgctgttctgtttgaaaataagtatggacttacaagaatgaaaagtcttcaacacttggaatatgttttcaataaagatcaggcctctcatctataataat

SEQ ID NO: 3 - DNA, 15 bp, Artificial
  synthetic oligonucleotide
attaggtccgtcagt

SEQ ID NO: 4 - DNA, 16 bp, Artificial
  synthetic oligonucleotide
ttacatgatgaccttt

SEQ ID NO: 5 - DNA, 21 bp, Artificial
  synthetic oligonucleotide
gccattgcaggaaatataaga

SEQ ID NO: 6 - DNA, 25 bp, Artificial
  CAGCTCTCAATTCTGTGAAACTCAA
cagctctcaattctgtgaaactcaa

SEQ ID NO: 7 - DNA, 20 bp, Artificial
  synthetic oligonucleotide
gcgactatacgcgcaatatg

SEQ ID NO: 8 - DNA, 20 bp, Artificial
  synthetic oligonucleotide
ctccagctccccccggcaac

SEQ ID NO: 9 - DNA, 21 bp, Artificial
  synthetic oligonucleotide
aactttcgatggtagtcgccg

SEQ ID NO: 10 - DNA, 21 bp, Artificial
  synthetic oligonucleotide
ccttggatgtggtagccgttt

SEQ ID NO: 11 - DNA, 20 bp, Artificial
  synthetic oligonucleotide
gtgggatatgacagggacgc

SEQ ID NO: 12 - DNA, 20 bp, artificial
  synthetic oligonucleotide
ggcaggagcagctaatccaa

SEQ ID NO: 13 - DNA, 20 bp, artificial
  synthetic oligonucleotide
aagcccaagaggctttccct

SEQ ID NO: 14 - DNA, 25 bp, artificial
  synthetic oligonucleotide
attattatagatgagaggcctgatc

SEQ ID NO: 15 - DNA, 19 bp, artificial
  synthetic oligonucleotide
gagtggtaagaccaccacc

SEQ ID NO: 16 - DNA, 20 bp, artificial
  synthetic oligonucleotide
ggttttcctcaggatctaat

SEQ ID NO: 17 - DNA, 22 bp, artificial
  synthetic oligonucleotide
agtgctatctgaacctgtggtc

SEQ ID NO: 18 - DNA, 20 bp, Artificial
  synthetic oligonucleotide
agtgctgggtcatccaaagg

SEQ ID NO: 19 - DNA, 20 bp, Artificial
  synthetic oligonucleotide
ctccgcacggaccatgtatt

SEQ ID NO: 20 - DNA, 20 bp, Artificial
  synthetic oligonucleotide
tcggactgaacgtggaaagg

SEQ ID NO: 21 - DNA, 20 bp, Artificial
  synthetic oligonucleotide
ccagagcagcatgagtggat

SEQ ID NO: 22 - DNA, 20 bp, Artificial
  synthetic oligonucleotide
tagctgctgttcactgctcc

SEQ ID NO: 23 - DNA, 20 bp, Artificial
  synthetic oligonucleotide
agcagacacacgtatgcgaa

SEQ ID NO: 24 - DNA, 20 bp, Artificial
  synthetic oligonucleotide
gtggttcccaatccccacat

SEQ ID NO: 25 - DNA, 20 bp, Artificial
  synthetic oligonucleotide
gtcctgacgacaacggacaa

SEQ ID NO: 26 - DNA, 20 bp, Artificial
  synthetic oligonucleotide
agaatgaagaccaaccgccc

SEQ ID NO: 27 - DNA, 20 bp, Artificial
  synthetic oligonucleotide
cacgtagacattccccgctt

SEQ ID NO: 28 - DNA, 20 bp, Artificial
  synthetic oligonucleotide
ctgcctgcgtaactagaggg

SEQ ID NO: 29 - DNA, 18 bp, Artificial
  synthetic oligonucleotide
gtgcctgcagccggcggg

SEQ ID NO: 30 - DNA, 42 bp, Artificial
  synthetic oligonucleotide
cttgcggccgctaaccgtctttcttctatttcagactcctcc

SEQ ID NO: 31 - DNA, 39 bp, Artificial
  synthetic oligonucleotide
cggttagcggccgcaagcaatgaagcatgcaggtctggg

SEQ ID NO: 32 - DNA, 31 bp, Artificial
  synthetic oligonucleotide
cttgtcatttcatgtctctttaattaaagag

SEQ ID NO: 33 - DNA, 28 bp, Artificial
  synthetic oligonucleotide
ggttaagcttagatgcatgctcgagcgg

SEQ ID NO: 34 - DNA, 37 bp, Artificial
  synthetic oligonucleotide
taatacgactcactatagggtgggtctggcacgaagg

SEQ ID NO: 35 - DNA, 18 bp, Artificial
  synthetic oligonucleotide
tgtgacggacgcaactgg

SEQ ID NO: 36 - DNA, 37 bp, Artificial
  synthetic oligonucleotide
taatacgactcactataggaatacatggtccgtgcgg

SEQ ID NO: 37 - DNA, 18 bp, Artificial
  synthetic oligonucleotide
cgcggcctgaggaagggg

SEQ ID NO: 38 - DNA, 37 bp, Artificial
  synthetic oligonucleotide
taatacgactcactataggcgggaggagcgcggaggc

SEQ ID NO: 39 - DNA, 20 bp, Artificial
  synthetic oligonucleotide
ccctagactcgccccacttg

SEQ ID NO: 40 - skipped

SEQ ID NO: 41 - DNA, 37 bp, Artificial
  synthetic oligonucleotide
taatacgactcactataggggacctaggcaacggcct

SEQ ID NO: 42 - DNA, 20 bp, Artificial
  synthetic oligonucleotide
accggcaggagcttcatcac

SEQ ID NO: 43 - DNA, 37 bp, Artificial
  synthetic oligonucleotide
taatacgactcactataggctttaatgccggggtctg

SEQ ID NO: 44 - DNA, 22 bp, Artificial
  synthetic oligonucleotide
aagctaaatatggacctacgct

SEQ ID NO: 45 - DNA, 38 bp, Artificial
  synthetic oligonucleotide
taatacgactcactataggagttttagaactggaaggg

SEQ ID NO: 46 - DNA, 25 bp, Artificial
  synthetic oligonucleotide
tctttcttctatttcagactcctcc

SEQ ID NO: 47 - DNA, 41 bp, Artificial
  synthetic oligonucleotide
taatacgactcactataggcaatgaagcatgcaggtctggg

SEQ ID NO: 48 - DNA, 30 bp, Artificial
  synthetic oligonucleotide
ctgcaaaagaaaacaacaaaaaaactaaag

SEQ ID NO: 49 - DNA, 68 bp, Artificial
  synthetic oligonucleotide
ctgggtggaagttgggggtgggacggcgccgggaggccggggaggcagcggcgaaaggcggcgggcgg

SEQ ID NO: 50 - DNA, 69 bp, Artificial
  synthetic oligonucleotide
attggcggggtctcggtgggggcgggccggttgccggggggagctggagtcacgtgacggccggcgcgg

SEQ ID NO: 51 - DNA, 69 bp, Artificial
  synthetic oligonucleotide
ccgctgtggctgtggtgggcggtaggggcttttaccgtgtgagctcctttaatgccggggtctgcagtg

SEQ ID NO: 52 - DNA, 69 bp, Artificial
  synthetic oligonucleotide
tgaatagaaggagggttgaggggatatggtgtggaatgggggaaggaggagtctgaaatagaagaaaga

SEQ ID NO: 53 - DNA, 2240 bp, Homo sapiens
catttcgtcttagccacgcagaagtcgcgtgtctaggtgagtcgcggtgggtcctcgcttgcagttcagcgaccacgtttgtttcgacgccggaccgcgtaagagacgatgatgttgggcacggaaggtggagagggattcgtggtgaaggtccggggcttgccctggtcttgctcggccgatgaagtgcagaggtttttttctgactgcaaaattcaaaatggggctcaaggtattcgtttcatctacaccagagaaggcagaccaagtggcgaggcttttgttgaacttgaatcagaagatgaagtcaaattggccctgaaaaaagacagagaaactatgggacacagatatgttgaagtattcaagtcaaacaacgttgaaatggattgggtgttgaagcatactggtccaaatagtcctgacacggccaatgatggctttgtacggcttagaggacttccctttggatgtagcaaggaagaaattgttcagttcttctcagggttggaaatcgtgccaaatgggataacattgccggtggacttccaggggaggagtacgggggaggccttcgtgcagtttgcttcacaggaaatagctgaaaaggctctaaagaaacacaaggaaagaatagggcacaggtatattgaaatctttaagagcagtagagctgaagttagaactcattatgatccaccacgaaagcttatggccatgcagcggccaggtccttatgacagacctggggctggtagagggtataacagcattggcagaggagctggctttgagaggatgaggcgtggtgcttatggtggaggctatggaggctatgatgattacaatggctataatgatggctatggatttgggtcagatagatttggaagagacctcaattactgtttttcaggaatgtctgatcacagatacggggatggtggctctactttccagagcacaacaggacactgtgtacacatgcggggattaccttacagagctactgagaatgacatttataattttttttcaccgctcaaccctgtgagagtacacattgaaattggtcctgatggcagagtaactggtgaagcagatgtcgagttcgcaactcatgaagatgctgtggcagctatgtcaaaagacaaagcaaatatgcaacacagatatgtagaactcttcttgaattctacagcaggagcaagcggtggtgcttacgaacacagatatgtagaactcttcttgaattctacagcaggagcaagcggtggtgcttatggtagccaaatgatgggaggcatgggcttgtcaaaccagtccagctacgggggcccagccagccagcagctgagtgggggttacggaggcggctacggtggccagagcagcatgagtggatacgaccaagttttacaggaaaactccagtgattttcaatcaaacattgcataggtaaccaaggagcagtgaacagcagctactacagtagtggaagccgtgcatctatgggcgtgaacggaatgggagggttgtctagcatgtccagtatgagtggtggatggggaatgtaattgatcgatcctgatcactgactcttggtcaaccttttttttttttttttttttttctttaagaaaacttcagtttaacagtttctgcaatacaagcttgtgatttatgcttactctaagtggaaatcaggattgttatgaagacttaaggcccagtatttttgaatacaatactcatctaggatgtaacagtgaagctgagtaaactataactgttaaacttaagttccagcttttctcaagttagttataggatgtacttaagcagtaagcgtatttaggtaaaagcagttgaattatgttaaatgttgccctttgccacgttaaattgaacactgttttggatgcatgttgaaagacatgcttttatttttttgtaaaacaatataggagctgtgtctactattaaaagtgaaacattttggcatgtttgttaattctagtttcatttaataacctgtaaggcacgtaagtttaagctttttttttttttaagttaatgggaaaaatttgagacgcaataccaatacttaggattttggtcttggtgtttgtatgaaattctgaggccttgatttaaatctttcattgtattgtgatttccttttaggtatattgcgctaagtgaaacttgtcaaataaatcctccttttaaaaactgca

SEQ ID NO: 54 - DNA, 38 bp, Homo sapiens
  misc_feature (11)..(11): n is a, c, g, or t
ttttttttttnggggctcggccggggagcgtgcggagt

SEQ ID NO: 55 - DNA, 45 bp, Homo sapiens
ttggaatatgttttcaataaagatcaggcctctcatctaaaaaaa

SEQ ID NO: 56 - DNA, 12 bp, Homo sapiens
aggcaggtaccc

SEQ ID NO: 57 - DNA, 12 bp, Homo sapiens
atttaggaagaa

(19) United States
(12) Patent Application Publication
(10) Pub. No.: US 2021/0380978 A1
(43) Pub. Date: Dec. 9, 2021

(54) The long non-coding RNA INCA1 and Homo sapiens heterogeneous nuclear ribonucleoprotein H1 (HNRNPH1) as therapeutic targets for immunotherapy

(71) Applicant: The Brigham and Women`s Hospital, Inc., Boston, MA (US)

(72) Inventors: Marco Mineo, Brighton, MA (US); E. Antonio Chiocca, Wakefield, MA (US)

(21) Appl. No.: 17/284,919

(22) PCT Filed: Oct. 15, 2019
(86) PCT No.: PCT/US2019/056362

§ 371 Date: Apr. 13, 2021

Related U.S. Application Data
(60) Provisional application No. 62/745,554, filed on Oct. 15, 2018.

(51) Int. Cl. C12N 15/113; A61K 45/06

(52) U.S. Cl. CPC C12N 15/113; A61K 45/06; C12N 2310/11; C12N 2310/3181; C12N 2310/315; C12N 2310/3231; C12N 2310/14

(57) ABSTRACT

Compositions comprising inhibitory nucleic acids targeting the long non-coding RNA INCA1, and methods of use thereof, e.g., in combination with immunotherapy, to treat cancer.

### CLAIM OF PRIORITY

This application claims the benefit of U.S. Provisional Application Ser. No. 62/745,554, filed on Oct. 15, 2018. The entire contents of the foregoing are incorporated herein by reference.

### FEDERALLY SPONSORED RESEARCH OR DEVELOPMENT

This invention was made with Government support under Grant Nos. CA163205 and CA069246 awarded by the National Institutes of Health. The Government has certain rights in the invention.

### TECHNICAL FIELD

Described herein are compositions comprising inhibitory nucleic acids targeting the long non-coding RNA INCA1 or Homo sapiens heterogeneous nuclear ribonucleoprotein H1 (HNRNPH1), and methods of use thereof, e.g., in combination with immunotherapy and/or chemotherapy, to treat cancer.

### BACKGROUND

Immune checkpoint inhibitors have revolutionized cancer treatment (Pardoll, 2012; Sharma and Allison, 2015). These therapies have been developed based on the ability of cancers to evade anti-tumor immunity by up-regulation of immune checkpoint molecules, such as programmed cell death 1 ligand 1 (PD-L1), in response to stimuli, such as interferon-γ (IFNγ) (Beatty and Gladney, 2015; Garcia-Diaz et al., 2017). Expression of PD-L1 within the tumor microenvironment inhibits the anti-tumor immune response through the binding of the immune checkpoint receptor PD-1 expressed on T cells (Freeman et al., 2000). Immune checkpoint inhibitors that target the PD-1/PD-L1 pathway have been shown to be less toxic than standard chemotherapy and to produce durable tumor regression and overall survival benefits in several tumors including non-small cell lung cancer (NSCLC) and melanoma (Antonia et al., 2017; Larkin et al., 2015; Topalian et al., 2012). However, only a small group of patients respond to these therapies and some of the responders develop acquired resistance (Jenkins et al., 2018). Moreover, immune checkpoint inhibitors have not produced significant benefits in tumors characterized by a highly immunosuppressive microenvironment, such as glioblastoma (GBM). Resistance to immune checkpoint blockade is partly caused by constitutive expression of interferon-stimulated genes (ISGs) in tumors, as a result of a persistent IFNγ signaling (Benci et al., 2016). Therefore, a better understanding of the molecular mechanisms that control IFNγ signaling and PD-L1 expression will benefit the development of alternative and more effective strategies to overcome present therapeutic limitations.

### SUMMARY

Interaction of PD-L1 with PD-1 suppresses T cell-mediated immunosurveillance. There is intense interest in identifying mechanisms that regulate this pathway because of recent therapeutic successes with immune checkpoint inhibitors. Here, we have identified the ubiquitous Interferon-γ (IFNγ)-stimulated Non-Coding RNA 1 (INCA1) as a long non-coding RNA (lncRNA) transcribed from the PD-L1 locus and show that INCA1 controls IFNγ signaling in multiple tumor types. Silencing INCA1 decreases the expression of PD-L1, JAK2 and several other IFNγ-stimulated genes. INCA1 knockdown sensitizes tumor cells to cytotoxic T cell-mediated killing, improving CAR T cell therapy. INCA1 regulation of PD-L1 and JAK2 is dependent on its interaction with HNRNPH1, a nuclear ribonucleoprotein identified as a negative regulator of PD-L1. A region of the INCA1 primary transcript functions as a decoy that acts in cis by binding to HNRNPH1, liberating PD-L1 and JAK2 transcripts and enabling their expression. Together, these results unveil a mechanism of tumor IFNγ signaling regulation mediated by the lncRNA INCA1. INCA1 is thus a new target for cancer immunotherapy.

Thus, provided herein are isolated inhibitory nucleic acids, i.e., inhibitory nucleic acids targeting INCA1, wherein the inhibitory nucleic acid comprises a sequence of nucleotides that are identical or complementary to 10 to 50 consecutive nucleotides of SEQ ID NO:1, or an isolated inhibitory nucleic acid targeting HNRNPH1, wherein the inhibitory nucleic acid comprises a sequence of nucleotides that are identical or complementary to 10 to 50 consecutive nucleotides of SEQ ID NO:53, as well as compositions comprising the same and/or methods of use thereof.

In some embodiments, the inhibitory nucleic acid is an antisense oligo (ASO), gapmer, mixmer, shRNA, or siRNA. In some embodiments, the inhibitory nucleic acid is modified. In some embodiments, the inhibitory nucleic acid comprises one or more modified bonds or bases. In some embodiments, the inhibitory nucleic acid comprises one or more peptide nucleic acid (PNA) or locked nucleic acid (LNA) molecules. In some embodiments, at least one nucleotide of the inhibitory nucleic acid is a ribonucleic acid analogue comprising a ribose ring having a bridge between its 2′-oxygen and 4′-carbon. In some embodiments, the ribonucleic acid analogue comprises a methylene bridge between the 2′-oxygen and the 4′-carbon. In some embodiments, at least one nucleotide of the inhibitory nucleic acid comprises a modified sugar moiety selected from a 2′-O-methoxyethyl modified sugar moiety, a 2′-methoxy modified sugar moiety, a 2′-O-alkyl modified sugar moiety, and a bicyclic sugar moiety. In some embodiments, the inhibitory nucleic acid comprises at least one modified internucleoside linkage selected from phosphorothioate, phosphorodithioate, alkylphosphonothioate, phosphoramidate, carbamate, carbonate, phosphate triester, acetamidate, carboxymethyl ester, and combinations thereof. In some embodiments, the inhibitory nucleic acid is configured such that hybridization of the inhibitory nucleic acid to the INCA1 or HNRNPH1 activates an RNAse H pathway in the cell; induces substantial cleavage or degradation of the INCA1 or HNRNPH1 RNA in the cell; or interferes with interaction of the INCA1 RNA with HNRNPH1 in the cell.

Further provided herein are compositions comprising the inhibitory nucleic acids described herein, and a pharmaceutically acceptable carrier.

Also provided herein are methods of treating a subject who has cancer, and the inhibitory nucleic acids described herein for use in a method of treating a subject who has cancer. The methods include administering to the subject a therapeutically effective amount of an inhibitory nucleic acid as described herein. In some embodiments, the methods include administering a therapeutically effective amount of an immunotherapy and/or a chemotherapeutic agent. In some embodiments, the immunotherapy comprises administration of an immune checkpoint inhibitor and/or chimeric antigen receptor (CAR)-expressing immune effector cells. In some embodiments, the CAR-expressing immune effector cells are T cells or NK cells. In some embodiments, the CAR-expressing immune effector cells are autologous to the subject. In some embodiments, the immune checkpoint inhibitor is an or comprises one or more anti-CD137 antibodies; anti-PD-1 (programmed cell death 1) antibodies; anti-PDL1 (programmed cell death ligand 1) antibodies; anti-PDL2 antibodies; or anti-CTLA-4 antibodies. In some embodiments, the subject has melanoma, breast, lung, colon, or brain cancer.

The present methods to prevent, treat or slow the progress of a pathological condition or disease in an animal can include the administration to said animal of a therapeutically effective amount of an agent, or a gene technology intervention that induces or promotes immune system activation in said animal. In some embodiments, the agent or gene technology intervention induces or promotes immune system activation by regulating the expression of an immune checkpoint ligand. In some embodiments, the immune checkpoint ligand is inducible by interferon-γ. In some embodiments, the interferon-γ inducible-ligand is PD-L1. In some embodiments, the therapeutically effective agent or gene technology intervention inhibits the induction, expression, or function of a long non-coding RNA (lncRNA). In some embodiments, the lncRNA is nuclear. In some embodiments, the nuclear lncRNA is lncRNA Interferon-γ-stimulated Non-Coding RNA 1 (INCA1). In some embodiments, the agent is (but not limited to) a small molecule, an antibody, a peptide or an agent associated with RNAi therapeutics. In some embodiments, the agent associated with RNAi therapeutics is an siRNA or an antisense oligonucleotide. In some embodiments, the siRNA or antisense oligonucleotide inhibits the expression or activity of a lncRNA. In some embodiments, the lncRNA is INCA1. In some embodiments, the antibody, peptide or small molecule inhibits the interaction or association of a lncRNA with one or more target molecules. In some embodiments, the lncRNA is INCA1. In some embodiments, the target molecule is a protein. In some embodiments, the INCA1 target protein is HNRNPH1. In some embodiments, the gene technology intervention is (but not limited to) a gene editing technology. In some embodiments, the gene editing technology system is based on zinc finger nucleases, TALENS, or CRISPR-Cas. In some embodiments, the gene technology intervention is used to decrease or silence lncRNA gene expression by modulating the lncRNA gene itself or by silencing the site-specific integration of RNA-destabilizing elements.

Also provided herein is a cellular composition comprised of a T cell genetically modified to express Chimeric Antigen Receptors (CAR T cells) wherein the CAR T cells comprise an antigen-binding domain, a transmembrane domain, a costimulatory signaling region, and an expression vector or gene insert encoding an siRNA or an antisense oligonucleotide. In some embodiments, the siRNA or antisense oligonucleotide inhibits the expression or activity of a lncRNA. In some embodiments, the lncRNA is INCA1.

Further provided herein are methods to prevent, treat or slow the progress of a pathological condition or disease in an animal characterized by, or exacerbated by, T cell exhaustion comprised of administering to said animal an effective amount of T cells genetically modified ex vivo to express Chimeric Antigen Receptors (CAR T cells) wherein the CAR T cells comprise an antigen binding domain, a transmembrane domain, a costimulatory signaling region, and an expression vector or gene insert encoding an siRNA or an antisense oligonucleotide. In some embodiments, the siRNA or antisense oligonucleotide inhibits the expression or activity of a lncRNA. In some embodiments, the lncRNA is INCA1. In some embodiments, the pathological condition or disease is cancer. In some embodiments, the cancer includes (but is not limited to) glioblastoma, non-small cell lung cancer, breast cancer, or melanoma.

In some embodiments, the animal is a mammal, e.g., a human.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and Figures, and from the claims.

### DESCRIPTION OF DRAWINGS

FIGS. 1A-I. INCA1 expression is induced by IFNγ and correlates with PD-L1. (A) Volcano plot of differentially expressed lncRNAs (RNA-seq) between unstimulated patient-derived GBM cell lines (PDGCLs) and PDGCLs stimulated with IFNγ (n=3 biological replicates). A set of the most up-regulated lncRNAs is annotated. 15,768 lncRNAs were surveyed in the analysis. (B) Schematic representation of the INCA1 gene and the genes within the same locus that are transcribed from the opposite strand. (C) Heat-map showing copy number losses and gains in GBM tumors and cell lines models (LTGCLs and PDGCLs). Horizontal-axis represents genetic markers along cytobands 9p24.1 to 9p21.1 while vertical-axis includes specimens (rows) stratified by groups. (D-F) qRT-PCR analysis of INCA1 (D) and PD-L1 (E) expression in unstimulated or IFNγ-stimulated PDGCLs, and correlation of INCA1 expression with PD-L1 expression in IFNγ-stimulated PDGCLs (F). R²=0.9991 calculated using linear regression analysis. Data shown as mean±s.e.m. of three biological replicates. Data were analyzed by unpaired t-test: *P<0.05, **P<0.01, ***P<0.001 (G-I) Expression of INCA1 (G) and PD-L1 (H) in GBM patient tumor specimens, and correlation of INCA1 expression with PD-L1 expression (I). R²=0.4087 calculated using linear regression analysis. Data shown as mean±s.d. of triplicates.

FIGS. 2A-L. INCA1 regulates tumor IFNγ signaling (A) Number of differentially expressed genes in INCA1 knockdown cells relative to control cells unstimulated or stimulated with IFNγ. (B) Gene ontology analysis of genes downregulated in IFNγ-treated INCA1 knockdown cells compared to control cells. (C) Heatmap of the expression levels of IFNγ-stimulated genes that were significantly downregulated in INCA1 knockdown cells compared to control. (D-H) qRT-PCR analysis of INCA1 (D), PD-L1 (E), PD-L2 (F), JAK2 (G), STAT1 (H) and IDO1 (I) expression in control or two independent INCA1-knockdown U251 cells unstimulated or stimulated with IFNγ. (J) Western blot analysis of PD-L1, JAK2 and IDO1 expression in control or two independent INCA1-knockdown U251 cells unstimulated or stimulated with IFNγ. (K) Flow cytometry analysis of cell surface levels of PD-L1 in control or two independent INCA1-knockdown U251 cells unstimulated or stimulated with IFNγ. (L) qRT-PCR analysis of INCA1 (A) and PD-L1 (B) expression in BT333 cells transfected with GapmeR negative control (NC) or two different GapmeRs targeting INCA1 (GapmeR 1 and GapmeR 2). Error bar represents mean SD (***p<0.001, ****p<0.0001).

FIGS. 3A-H. Silencing INCA1 leads to increased T cell-mediated cytotoxicity in vitro and improves CAR T cell efficacy in vivo. (A) ELISA analysis of IFNγ secretion from non-activated or activated CD8⁺ T cells co-cultured with control or INCA1-knockdown U251 cells for 48 h. (B) ATP quantitation-based cell viability analysis of control and INCA1-knockdown U251 cells co-cultured with activated CD8⁺ T cells for 96 h. Data are normalized to the viability of corresponding cells co-cultured with non-activated CD8⁺ T cells. (C) GFP-positive control and INCA1-knockdown U251 tumorspheres were co-cultured with non-activated or activated CD8⁺ T cells and T cell cytotoxic activity was evaluated at 96 h. Shown are representative fluorescent microscopy pictures (left) and area of tumorspheres co-cultured with activated CD8⁺ T cells (right). (D) NSG mice were injected with U251-EGFRvIII shControl (black and green lines) or shINCA1 (blue and red lines) subcutaneously on day 0 and with T cells intravenously on day 7 (black arrow). Tumor volume was measured over time. (E) Flow cytometry analysis of CD4/CD8 composition of EGFRvIII specific CAR T cells infiltrating control (left) and INCA1-knockdown (right) tumors. (F-G) Flow cytometry analysis of the number of CD4⁺ (F) and CD8⁺ (G) CAR T cells in control and INCA1-knockdown tumors 21 days post intravenous injection of T cells. (H) Flow cytometry analysis of PD-1 levels in CD8⁺ CAR T cells isolated from control and INCA1-knockdown. Error bar represents mean±SD (*p<0.05, **p<0.01, ***p<0.001). Tumor volume data were analyzed by two-way ANOVA (****p<0.001).

FIGS. 4A-G. HNRNPH1 is a binding partner of INCA1. (A-B), qRT-PCR analysis of RNAs extracted from cytoplasmic and nuclear compartments of unstimulated (A) or IFNγ-stimulated (B) BT333 cells. MALAT1 and GAPDH were used to assess fractionation efficiency. Data are representative of three independent experiments and are shown as mean±s.d. (C) BT333 were stimulated with IFNγ and RNA captured using biotinylated probes antisense to INCA1 or scramble control probe was analyzed by qRT-PCR. Data shown as mean±s.d. of triplicates. (D) Top 10 proteins co-purified with INCA1 from RNA antisense purification (RAP). (E-F), HNRPNH1 RIP (RNA immunoprecipitation) followed by qRT-PCR analysis of co-purified INCA1 (E), PD-L1 (F, left), and JAK2 (F, right) in UV-crosslinked BT164 cells unstimulated or stimulated with IFNγ. Data shown as mean±s.d. of three independent experiments (****P<0.0001). (G) MDA-MB-231 cells were stimulated with IFNγ and RAP-RNA was performed using biotinylated probes antisense to INCA1 (yellow) or scramble. RNA co-purified with INCA1 was analyzed by qRT-PCR. Error bar represents mean±SD (****p<0.0001).

FIGS. 5A-L. INCA1 functions as a negative regulator of HNRNPH1 activity. (A-B) qRT-PCR analysis of HNRNPH1 (A), PD-L1 (B, left) and JAK2 (B, right) expression in unstimulated or IFNγ-stimulated A375 cells transfected with siRNA control or two different siRNAs targeting HNRNPH1. (C) Western blot analysis of PD-L1, JAK2 and HNRNPH1 expression in control or two independent HNRNPH1-knockdown A375 cells unstimulated or stimulated with IFNγ. (D) Correlation of HNRNPH1 expression with PD-L1 expression in GBM tumors. (E) Control and INCA1-knockdown A375 cells were transfected with siRNA control or two independent siRNAs targeting HNRNPH1. Cells were stimulated with IFNγ and expression of HNRNPH1 and PD-L1 was analyzed by Western blot. (F) Identification of HNRNPH1 binding sites in INCA1 (top), PD-L1 (middle), and JAK2 (bottom) by eCLIP. Read density in reads per million (RPM) are shown for HNRNPH1, IgG and input. (G) Schematic representation of INCA1 minigene with eCLIP reads and RNA fragments (F1-7) covering the 5′ and 3′ regions of the INCA1 first intron (top); and RNA pull-down validation of INCA1 interaction with HNRNPH1 using the 7 different biotinylated RNA fragments (bottom). (H) Binding curves of HNRNPH1 interaction with a scrambled RNA (top) or a 50 nucleotide RNA oligo whose sequence represents the major eCLIP peak (bottom), demonstrating a specific binding of HNRNPH1 to the latter with a K_{d} of 762.11 nM. (I) RNA pull-down assay with biotinylated fragment 4 (F4) in the presence of antisense oligonucleotide control (ASO NC) or targeting HNRNPH1 binding site (ASO H1B). (J-K) qRT-PCR analysis of INCA1 (J) and PD-L1 (K) expression in unstimulated or IFNγ-stimulated A375 cells transfected with ASO NC or ASO H1B. (L) Western blot analysis of PD-L1 and JAK2 expression in ASO NC or ASO H1B transfected A375 cells unstimulated or stimulated with IFNγ. Data shown as mean±s.d. Data were analyzed by unpaired t-test: ****P<0.0001.

FIGS. 6A-B. LncRNAs correlated with INCA1 expression. (A) Approximately 237 lncRNAs were positively correlated and 1,188 negatively correlated (p-value<0.05, FDR<0.25) with INCA1 expression. (B) The Venn diagram shows the number of coding genes, transcribed from the same loci of lncRNAs correlated with INCA1, whose expression is regulated by one or more IFN type (Type I, II or III). Venn diagram was generated using the Interferome database (interferome.org).

FIGS. 7A-D. Characterization of the INCA1 sequence. (A-B) PCR products from 5′ and 3′ RACE were purified and sequenced. Shown are DNA sequencing traces for 5′ (A) and 3′ (B) RACE. (C) Full length INCA1 was PCR amplified and purified product was sequenced. Shown are DNA sequencing traces for exon junctions of the INCA1 expressed in patient derived GBM cell lines. (D) Genome Browser alignment of the INCA1 sequence.

FIG. 8. INCA1 is a non-coding RNA. INCA1 was in vitro transcribed and translated and reaction product was analyzed by Western blot (lane 3). Absence of protein product confirms INCA1 as a non-coding RNA. pSP64-Luciferase and pcDNA3.1-GFP vectors were used as positive control (lanes 1 and 4 respectively). No template reaction was used as negative control (lane 2).

M=Protein precision plus dual color standards

1=pSP64—Luciferase control DNA (61 kDa)

2=No template control

3=pcDNA3.1-INCA1

4=pcDNA3.1-GFP control (27 kDa)

FIGS. 9A-B. PD-L1 is expressed in patient derived GBM cell lines. (A) Western blot analysis of PD-L1 expression in PDGCLs unstimulated or stimulated with IFNγ. (B) qRT-PCR analysis of INCA1 and PD-L1 copy number in IFNγ-stimulated PDGCLs.

FIGS. 10A-E. INCA1 expression does not correlate with PD-L2 or RIC1 expression. (A-B) qRT-PCR analysis of PD-L2 (A) expression in unstimulated or IFNγ-stimulated PDGCLs, and correlation of INCA1 expression with PD-L2 expression in IFNγ-stimulated PDGCLs (B). R²=0.5239 calculated using linear regression analysis. Data shown as mean±s.e.m. of three biological replicates. (C-D) qRT-PCR analysis of PD-L2 (C) expression in GBM patient tumor specimens, and correlation of INCA1 expression with PD-L2 expression (D). R²=0.2085 calculated using linear regression analysis. Data shown as mean±s.d. of triplicates. (E) qRT-PCR analysis of RIC1 expression in unstimulated or IFNγ-stimulated PDGCLs. Data shown as mean±s.e.m. of three biological replicates.

FIGS. 11A-D. INCA1 is expressed in different tumor types. (A-D) qRT-PCR analysis of INCA1 (A) and PD-L1 (B) expression in unstimulated or IFNγ-stimulated long term cell lines from different tumor types and correlation of INCA1 expression with PD-L1 expression in unstimulated (C) and IFNγ-stimulated cells (D). Tumor cell types include glioblastoma (GBM), melanoma, non-small cell lung cancer (NSCLC) and breast cancer (BC). R² calculated using linear regression analysis. Data shown as mean±s.e.m. of three biological replicates.

FIGS. 12A-H. INCA1 regulates PD-L1 expression in different tumor types. (A-B) qRT-PCR analysis of INCA1 (A) and PD-L1 (B) expression in control or two independent INCA1-knockdown A375 melanoma cells unstimulated or stimulated with IFNγ. (C) Western blot analysis of PD-L1 expression in control or two independent INCA1-knockdown A375 melanoma cells unstimulated or stimulated with IFNγ. (D) Flow cytometry analysis of cell surface levels of PD-L1 in control or two independent INCA1-knockdown A375 melanoma cells unstimulated or stimulated with IFNγ. (E-F) qRT-PCR analysis of INCA1 (E) and PD-L1 (F) expression in control or two independent INCA1-knockdown MDA-MB-231 breast cancer cells unstimulated or stimulated with IFNγ. (G) Western blot analysis of PD-L1 expression in control or two independent INCA1-knockdown MDA-MB-231 breast cancer cells unstimulated or stimulated with IFNγ. (H) Flow cytometry analysis of cell surface levels of PD-L1 in control or two independent INCA1-knockdown MDA-MB-231 breast cancer cells unstimulated or stimulated with IFNγ. Data are representative of two (A-H) independent experiments. Data shown as mean±s.d. of triplicates (A, B, D, E, F, H). Data were analyzed by unpaired t-test: ***P<0.001, ****P<0.0001.

FIGS. 13A-C. INCA1 promotes tumor cell escape from T cell-mediated killing in different tumor types. (A-C) Flow cytometry analysis of cell viability in control and INCA1-knockdown U251 (A), A375 (B) and MDA-MB-231 (C) cells co-cultured with activated CD8⁺ T cells for 96 h. Data are representative of two independent experiments. Data are shown as mean±s.d. of 4 replicates and analyzed by unpaired t-test: ***P<0.001.

FIGS. 14A-F. Validation of HNRNPH1 RNA-immunoprecipitation. (A-D) HNRPNH1 RIP followed by qRT-PCR analysis of co-purified RNAs in UV-crosslinked BT164, BT245 and BT333 cells unstimulated or stimulated with IFNγ. MALAT1 (A) and NORAD (B) lncRNAs were used as positive control of HNRNPH1 binding. RMRP (C) and 18S (D) lncRNAs were used as negative control. Data are shown as mean±s.d. of triplicates. (E) qRT-PCR analysis of the expression of the lncRNAs MALAT1, NORAD and RMRP in PDGCLs. Data shown as mean±s.d. of Ct values. (F) qRT-PCR analysis of RNAs extracted from cytoplasmic and nuclear compartments of IFNγ-stimulated PDGCLs. Data are shown as mean±s.d.

FIGS. 15A-F. HNRNPH1 regulates PD-L1 and JAK2 expression in an INCA1-dependent manner. (A-C) qRT-PCR analysis of HNRNPH1 (A), PD-L1 (B, top), JAK2 (B, bottom), and INCA1 (C) expression in unstimulated or IFNγ-stimulated U251 cells transfected with siRNA control or two different siRNAs targeting HNRNPH1. (D) qRT-PCR analysis of INCA1 expression in unstimulated or IFNγ-stimulated A375 cells transfected with siRNA control or two different siRNAs targeting HNRNPH1. (E) Western blot analysis of PD-L1 and HNRNPH1 expression in control or two independent HNRNPH1-knockdown U251 cells unstimulated or stimulated with IFNγ. (F) Control and INCA1-knockdown U251 cells were transfected with siRNA control or two independent siRNAs targeting HNRNPH1. Cells were stimulated with IFNγ and HNRNPH1 and PD-L1 expression analyzed by Western blot. Data are representative two independent experiments (A-F). Data shown as mean±s.d. of triplicates (A-D). Data were analyzed by unpaired t-test: *P<0.05, **P<0.01, ****P<0.0001.

FIGS. 16A-B. HNRNPH1 binding to INCA1 is inhibited by antisense oligonucleotide. (A) EMSA analysis of HNRNPH1 binding to radiolabeled oligonucleotide (50 bases) whose sequence represents the major eCLIP peak. The highest protein concentration used was 350 μM, and 2-fold serial dilutions were assayed. No protein was added to the lane marked “0”. (B) RNA pull-down analysis of biotinylated fragment 4 (F4) in the presence of increasing concentrations of antisense oligonucleotide targeting HNRNPH1 binding site (ASO H1B). No RNA fragment was added in the lanes marked “-”. Data are representative of three independent experiments.

### DETAILED DESCRIPTION

Long non-coding RNAs (lncRNAs), transcripts longer than 200 nucleotides that lack protein coding potential, have emerged as major regulators of a wide range of cellular processes (10, 16). LncRNAs are involved in several biological processes, including cell proliferation, migration and adaptation to stress (11-13). De-regulation of lncRNA expression has been shown to drive cancer progression (14). However, lncRNAs have not yet been implicated in tumor immune evasion.

A poorly characterized lncRNA, referred to herein as Interferon-γ-stimulated Non-Coding RNA 1 (INCA1), is shown herein to be a major regulator of IFNγ signaling in tumors by in-cis post-transcriptional modulation of PD-L1 and JAK2 expression. Unexpectedly, the primary INCA1 transcript, and not the mature lncRNA, modulates the activity of an RNA binding protein, HNRNPH1, to affect PD-L1 and JAK2 levels. INCA1 is transcribed as an antisense RNA from the PD-L1/PD-L2 locus and its expression strongly correlates with PD-L1 but not PD-L2 expression. INCA1 is expressed in human patients and across multiple tumor types, and its levels increase after IFNγ stimulation. As shown herein, silencing INCA1 represses the expression of ISGs, including PD-L1, in both control and IFNγ-stimulated cells. Furthermore, INCA1 knockdown cells are more susceptible to cytotoxic T cell-mediated killing compared to control cells. In vivo, silencing INCA1 resulted in increased susceptibility to CAR T cell therapy in an experimental tumor model. Finally, INCA1 functions as a decoy RNA to sequester HNRNPH1, a member of the heterogeneous ribonucleoprotein family that binds PD-L1 and JAK2 transcripts to negatively regulate their expression. Together, the present data reveal a mechanism of interferon signaling regulation mediated by the lncRNA INCA1. As shown herein, targeting INCA1 is a valid strategy to downregulate multiple immunosuppressive molecules and improve cancer immunotherapy.

### Methods of Treatment

In view of the discovery that targeting INCA1 or hnRPH1 is a valid strategy to downregulate multiple immunosuppressive molecules and improve cancer immunotherapy, provided herein are methods of treating a cancer in a subject that include administering an inhibitory nucleic acid targeting INCA1 or hnRPH1 as described herein, optionally in combination with an immunotherapy. Specific embodiments and various aspects of these methods are described below.

Methods of Treating Cancer

The methods generally include identifying a subject who has a tumor, e.g., a cancer. As used herein, the term “cancer” refers to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. In general, a cancer will be associated with the presence of one or more tumors, i.e., abnormal cell masses. The term “tumor” is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. While the present study focused on pancreatic cancer because of its dismal prognosis and the lack of progress against its metastatic form, the present compositions and methods are broadly applicable to solid malignancies. Thus the cancer can be of any type of solid tumor, including but not limited to: breast, colon, kidney, lung, skin, ovarian, pancreatic, rectal, stomach, thyroid, or uterine cancer.

Tumors include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In some embodiments, cancers evaluated or treated by the methods described herein include epithelial cancers, such as a lung cancer (e.g., non-small-cell lung cancer (NSCLC)), breast cancer, colorectal cancer, kidney cancer, head and neck cancer, prostate cancer, pancreatic cancer (e.g., Pancreatic ductal adenocarcinoma (PDAC)) or ovarian cancer. Epithelial malignancies are cancers that affect epithelial tissues.

A cancer can be diagnosed in a subject by a health care professional (e.g., a physician, a physician's assistant, a nurse, or a laboratory technician) using methods known in the art. For example, a metastatic cancer can be diagnosed in a subject, in part, by the observation or detection of at least one symptom of a cancer in a subject as known in the art. A cancer can also be diagnosed in a subject using a variety of imaging techniques (e.g., alone or in combination with the observance of one or more symptoms of a cancer in a subject). For example, the presence of a cancer can be detected in a subject using computer tomography, magnetic resonance imaging, positron emission tomography, and X-ray. A cancer can also be diagnosed by performing a biopsy of tissue from the subject. A cancer can also be diagnosed from serum biomarkers, such as CA19.9, CEA, PSA, etc.

In some embodiments, the methods can include determining whether the cancer expresses or overexpresses an immune checkpoint molecule, e.g., PD-L1. Methods for detecting expression of an immune checkpoint molecule, e.g., PD-L1 in a cancer, e.g., in a biopsy or other sample comprising cells from the cancer, are known in the art, e.g., including commercially available or laboratory-developed immunohistochemistry (IHC); see, e.g., Udall et al., Diagn Pathol. 2018; 13: 12. The level can be compared to a threshold or reference level, and if a level of expression of an immune checkpoint molecule, e.g., PD-L1 above the threshold or reference level are seen, the subject can be selected for a treatment as descried herein. In some embodiments, the methods can include determining whether the cancer has high levels of microsatellite instability (MSI), e.g., as described in Kawakami et al., Curr Treat Options Oncol. 2015 July; 16(7):30; Zeinalian et al., Adv Biomed Res. 2018; 7: 28, and selecting for treatment a cancer that is MSI-high or that has levels of MSI above a threshold or reference level.

A treatment comprising any one or more of the inhibitory nucleic acids targeting INCA1 or hnRPH1 as described herein, optionally in combination with an immunotherapy, as described herein, can be administered to a subject having cancer. The treatment can be administered to a subject in a health care facility (e.g., in a hospital or a clinic) or in an assisted care facility. In some embodiments, the subject has been previously diagnosed as having a cancer. In some embodiments, the subject has already received therapeutic treatment for the cancer. In some embodiments, one or more tumors has been surgically removed prior to treatment as described herein.

In some embodiments, the administering of at least one inhibitory nucleic acid targeting INCA1 or hnRPH1 as described herein, optionally in combination with an immunotherapy, results in a decrease (e.g., a significant or observable decrease) in the size of a tumor, a stabilization of the size (e.g., no significant or observable change in size) of a tumor, or a decrease (e.g., a detectable or observable decrease) in the rate of the growth of a tumor present in a subject. A health care professional can monitor the size and/or changes in the size of a tumor in a subject using a variety of different imaging techniques, including but not limited to: computer tomography, magnetic resonance imaging, positron emission tomography, and X-ray. For example, the size of a tumor of a subject can be determined before and after therapy in order to determine whether there has been a decrease or stabilization in the size of the tumor in the subject in response to therapy. The rate of growth of a tumor can be compared to the rate of growth of a tumor in another subject or population of subjects not receiving treatment or receiving a different treatment. A decrease in the rate of growth of a tumor can also be determined by comparing the rate of growth of a tumor both prior to and following a therapeutic treatment (e.g., treatment with any of the inhibitory nucleic acids targeting INCA1 or hnRPH1 as described herein, optionally in combination with an immunotherapy, as described herein). In some embodiments, the visualization of a tumor can be performed using imaging techniques that utilize a labeled probe or molecule that binds specifically to the cancer cells in the tumor (e.g., a labeled antibody that selectively binds to an epitope present on the surface of the cancer cell).

In some embodiments, administering an inhibitory nucleic acid targeting INCA1 or hnRPH1 as described herein, optionally in combination with an immunotherapy, to the subject decreases the risk of developing a metastatic cancer (e.g., a metastatic cancer in a lymph node) in a subject having (e.g., diagnosed as having) a primary cancer (e.g., a primary breast cancer) (e.g., as compared to the rate of developing a metastatic cancer in a subject having a similar primary cancer but not receiving treatment or receiving an alternative treatment). A decrease in the risk of developing a metastatic tumor in a subject having a primary cancer can also be compared to the rate of metastatic cancer formation in a population of subjects receiving no therapy or an alternative form of cancer therapy.

A health care professional can also assess the effectiveness of therapeutic treatment of a cancer by observing a decrease in the number of symptoms of cancer in the subject or by observing a decrease in the severity, frequency, and/or duration of one or more symptoms of a cancer in a subject. A variety of symptoms of a cancer are known in the art and are described herein.

In some embodiments, the administering can result in an increase (e.g., a significant increase) in lifespan or chance of survival or of a cancer in a subject (e.g., as compared to a population of subjects having a similar cancer but receiving a different therapeutic treatment or no therapeutic treatment). In some embodiments, the administering can result in an improved prognosis for a subject having a cancer (e.g., as compared to a population of subjects having a similar cancer r but receiving a different therapeutic treatment or no therapeutic treatment).

Dosing, Administration, and Compositions

In any of the methods described herein, the inhibitory nucleic acid targeting INCA1 or hnRPH1 as described herein, optionally in combination with an immunotherapy, can be administered by a health care professional (e.g., a physician, a physician's assistant, a nurse, or a laboratory or clinic worker), the subject (i.e., self-administration), or a friend or family member of the subject. The administering can be performed in a clinical setting (e.g., at a clinic or a hospital), in an assisted living facility, or at a pharmacy.

In some embodiments of any of the methods described herein, the inhibitory nucleic acid targeting INCA1 or hnRPH1 as described herein, optionally in combination with an immunotherapy, is administered to a subject that has been diagnosed as having a cancer. In some embodiments, the subject has been diagnosed with brain cancer, e.g., GBM; breast cancer; or pancreatic cancer. In some non-limiting embodiments, the subject is a man or a woman, an adult, an adolescent, or a child. The subject can have experienced one or more symptoms of a cancer or metastatic cancer (e.g., a metastatic cancer in a lymph node). The subject can also be diagnosed as having a severe or an advanced stage of cancer (e.g., a primary or metastatic cancer). In some embodiments, the subject may have been identified as having a metastatic tumor present in at least one lymph node. In some embodiments, the subject may have already undergone surgical resection, e.g., partial or total pancreatectomy, lymphectomy and/or mastectomy.

In some embodiments of any of the methods described herein, the subject is administered at least one (e.g., at least 2, 3, 4, 5, 6, 7, 8, 9, 10, 15, 20, 25, or 30) dose of a composition containing at least one (e.g., one, two, three, or four) of any of the inhibitory nucleic acids targeting INCA1 or hnRPH1 as described herein, optionally in combination with an immunotherapy, or pharmaceutical compositions described herein. In any of the methods described herein, the at least one inhibitory nucleic acids or pharmaceutical composition (e.g., any of the inhibitory nucleic acids or pharmaceutical compositions described herein) can be administered intravenously, intraarterially, subcutaneously, intraperitoneally, or intramuscularly to the subject. In some embodiments, the at least one inhibitory nucleic acids or pharmaceutical composition is directly administered (injected) into a lymph node in a subject.

In some embodiments, the subject is administered at least one inhibitory nucleic acids targeting INCA1 or hnRPH1 as described herein, optionally in combination with an immunotherapy, or pharmaceutical composition (e.g., any of the inhibitory nucleic acids targeting INCA1 or hnRPH1 as described herein, optionally in combination with an immunotherapy or pharmaceutical compositions described herein) and at least one additional therapeutic agent. The at least one additional therapeutic agent can be a chemotherapeutic agent. By the term “chemotherapeutic agent” is meant a molecule that can be used to reduce the rate of cancer cell growth or to induce or mediate the death (e.g., necrosis or apoptosis) of cancer cells in a subject (e.g., a human). In non-limiting examples, a chemotherapeutic agent can be a small molecule, a protein (e.g., an antibody, an antigen-binding fragment of an antibody, or a derivative or conjugate thereof), a nucleic acid, or any combination thereof. Non-limiting examples of chemotherapeutic agents include one or more alkylating agents; anthracyclines; cytoskeletal disruptors (taxanes); epothilones; histone deacetylase inhibitors; inhibitors of topoisomerase I; inhibitors of topoisomerase II; kinase inhibitors; nucleotide analogs and precursor analogs; peptide antibiotics; platinum-based agents; retinoids; and/or vinca alkaloids and derivatives; or any combination thereof. In some embodiments, the chemotherapeutic agent is a nucleotide analog or precursor analog, e.g., azacitidine; azathioprine; capecitabine; cytarabine; doxifluridine; fluorouracil; gemcitabine; hydroxyurea; mercaptopurine; methotrexate; or tioguanine. Other examples include cyclophosphamide, mechlorethamine, chlorabucil, melphalan, daunorubicin, doxorubicin, epirubicin, idarubicin, mitoxantrone, valrubicin, paclitaxel, docetaxel, etoposide, teniposide, tafluposide, bleomycin, carboplatin, cisplatin, oxaliplatin, all-trans retinoic acid, vinblastine, vincristine, vindesine, vinorelbine, and bevacizumab (or an antigen-binding fragment thereof). Additional examples of chemotherapeutic agents are known in the art.

In some embodiments, the chemotherapeutic agent is chosen based on the cancer type or based on genetic analysis of the cancer; for example, for pancreatic cancer, one or more of ABRAXANE (albumin-bound paclitaxel), Gemzar (gemcitabine), capecitabine, 5-FU (fluorouracil) and ONIVYDE (irinotecan liposome injection), or combinations thereof, e.g., FOLFIRINOX, a combination of three chemotherapy drugs (5-FU/leucovorin, irinotecan and oxaliplatin), or modified FOLFIRINOX (mFOLFIRINOX) can be administered. Further combinations of targets that may work synergistically by complementary mechanisms could be used. For example, combination therapies can be used that physically alter the tumor microenviroment by enzymatic degradation via recombinant human hyaluronidase (PEGPH20),^{30,31} or other alternative chemotherapy agents, and/or alternative checkpoint inhibitors that may promote a synergistic effect in activating T-cells (e.g., anti-PD-1 and/or anti-CTLA-4).

The methods and compositions can also include administration of an analgesic (e.g., acetaminophen, diclofenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, phenylbutazone, piroxicam, sulindac, tolmetin, celecoxib, buprenorphine, butorphanol, codeine, hydrocodone, hydromorphone, levorphanol, meperidine, methadone, morphine, nalbuphine, oxycodone, oxymorphone, pentazocine, propoxyphene, and tramadol).

In some embodiments, at least one additional therapeutic agent and at least one inhibitory nucleic acids targeting INCA1 or hnRPH1 as described herein, optionally in combination with an immunotherapy, are administered in the same composition (e.g., the same pharmaceutical composition). In some embodiments, the at least one additional therapeutic agent and the at least one inhibitory nucleic acid targeting INCA1 or hnRPH1 as described herein, optionally in combination with an immunotherapy, are administered to the subject using different routes of administration (e.g., at least one additional therapeutic agent delivered by oral administration and at least one inhibitory nucleic acids targeting INCA1 or hnRPH1 as described herein, optionally in combination with an immunotherapy, delivered by intravenous administration).

In any of the methods described herein, the at least one inhibitory nucleic acids targeting INCA1 or hnRPH1 as described herein, optionally in combination with an immunotherapy, and, optionally, at least one additional therapeutic agent can be administered to the subject at least once a week (e.g., once a week, twice a week, three times a week, four times a week, once a day, twice a day, or three times a day). In some embodiments, at least two different inhibitory nucleic acids targeting INCA1 or hnRPH1 as described herein, optionally in combination with an immunotherapy, are administered in the same composition (e.g., a liquid composition). In some embodiments, at least inhibitory nucleic acid targeting INCA1 or hnRPH1 as described herein, optionally in combination with an immunotherapy, and at least one additional therapeutic agent are administered in the same composition (e.g., a liquid composition). In some embodiments, the at least one inhibitory nucleic acids targeting INCA1 or hnRPH1 as described herein, optionally in combination with an immunotherapy, and the at least one additional therapeutic agent are administered in two, three or more different compositions (e.g., a liquid composition containing at least one inhibitory nucleic acids targeting INCA1 or hnRPH1 as described herein, in combination with or separate from the optional immunotherapy, and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to administering the at least one inhibitory nucleic acids targeting INCA1 or hnRPH1 as described herein, optionally in combination with an immunotherapy. In some embodiments, the one or more additional therapeutic agents can be administered to the subject after administering the at least one inhibitory nucleic acids targeting INCA1 or hnRPH1 as described herein, optionally in combination with an immunotherapy. In some embodiments, the one or more additional therapeutic agents and the at least one inhibitory nucleic acids targeting INCA1 or hnRPH1 as described herein, optionally in combination with an immunotherapy, are administered to the subject such that there is an overlap in the bioactive period of the one or more additional therapeutic agents and the inhibitory nucleic acids targeting INCA1 or hnRPH1 as described herein, and/or the optional immunotherapy, in the subject.

In some embodiments, the subject can be administered the at least one inhibitory nucleic acids targeting INCA1 or hnRPH1 as described herein, optionally in combination with an immunotherapy, over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, 5 years, or 10 years). A skilled medical professional may determine the length of the treatment period using any of the methods described herein for diagnosing or following the effectiveness of treatment (e.g., using the methods above and those known in the art). As described herein, a skilled medical professional can also change the identity and number (e.g., increase or decrease) of inhibitory nucleic acids targeting INCA1 or hnRPH1 as described herein, and/or optional immunotherapy, (and/or one or more additional therapeutic agents) administered to the subject and can also adjust (e.g., increase or decrease) the dosage or frequency of administration of at least one inhibitory nucleic acids targeting INCA1 or hnRPH1 as described herein, and/or optional immunotherapy, (and/or one or more additional therapeutic agents) to the subject based on an assessment of the effectiveness of the treatment (e.g., using any of the methods described herein and known in the art). A skilled medical professional can further determine when to discontinue treatment (e.g., for example, when the subject's symptoms are significantly decreased).

Inhibitory Nucleic Acids Targeting INCA1 or HNRNPH1

The compositions useful in the present methods can include one or more inhibitory nucleic acids targeting INCA lncRNA or heterogeneous nuclear ribonucleoprotein H1 (HNRNPH1).

An exemplary sequence for human INCA1 is NC_000009.12, Reference GRCh38.p7 Primary Assembly, Range 5452251-5629895 (complement).

In some embodiments, the sequence of human INCA1 is

(SEQ ID NO: 1)
GGGCTCGGCCGGGGAGCGTGCGGAGTCTGCACGCCGCGGCCGTCGGTCCA
ACGCGGGGAGGCTCAGGGATTTTCGGCAGCAGCGACTTCATCCACCCCGG
ACCGCCGGCTGGAGGACGTGCCTGCAGCCGGCGGGTACCTGCGGCCAATT
CGGTGGAACGAGGACGCGGACTGCGAAGGCAGGTACCCTCCTCTGAAATC
AAAACTCTTTAATTAAAGAGACATGAAATGACAAGATTTTAAAAAAATCC
CATGAGGAGAAGGCATTCTAATTTAGGAAGAACTCCCCCTTGGAAAAACC
ATCAGTGCCGGAAGATTTCCTATTGTGTTGATCCATGGCAAAGGAGACTG
CAGATACACAAGGGATATTATGGAGCCCAGACGACCTGAATAAAACCCTT
CCCTACTACAAGGACAGCTGTCCCTTCCCTACACACTCCCTACAGGCTGA
TGAGAGACCTTTTTTGGAAGCAGAAACTTATACTTTATGCTGCCTTCTTC
CTGACTGCCAGGATTATACTCTTCCTTTCCATCCCAGATCTAGCAATGCT
GTTGATGAGGCTAAGTCATGATGATTTCTTTAATATCTTGGAACACAGTA
GATGCCTGATATTTGCGGATGGACTGGAGAAAAACTGAAAGTATAAACCA
CAACATCTCAAGAGATGTCATGAATGGAGAAGCATATGGTAAAATATAAT
GAAAATTAAATCTACTTTACAAGTGGTATCTTCTTGACAATAGTGGCATT
ACCTGAGCCAGTATCAGGGACCAGAAATACAACATAAAACTGATACATTT
CTCTTAATCCCCACATGATTTTGTTTTTGAGTTTATAGTTATCAGTGATC
ATTGATTAATAATTGATCAGTGATCAGTGACAAATGTATACTTTTTCTAC
TTGTATTGTTAGCCCTCAGATTGATGGTAGTTAGCCCAGGAATGGAAAGA
CCAAGGGGAGATGATTTAATTCTAGTCAAATATTAAAAGGTCATCATGTA
AATGAAATAATCCCAAGACTACAACTAGAGGGGCAGATTTCAGCTTACTA
TTAGGGAGTAATTTCAACAGGATATGGAAGGGATATCCAAATGGCAGGGA
GGCCATTGTGGGATATGACAGGGACGCCATTGCAGGAAATATAAGAACAG
AAGATCCTCTGGGGGATTTTTGTTCTTTGTGGGAGTTTGGATTAGCTGCT
CCTGCCTGGGATCCTTTCAGCTCTCAATTCTGTGAAACTCAACAATGAAA
GCAGAGGTGTTTTCCTTTACTTAGCTAGTGCCAACACAAAACTTTGAACT
CAAGTGACCTGATAACATAACTGTGGGTCAATACCGAGGAACTAACACTC
CTAGTTCCTTTTCAATTCCCCAAGACAACATCAGTATAGATTTTCAAGAT
GAATATATGCCCAGTAAGTATGGCTTATCCTGTACTGGAAGAATAGGTTC
TTTCACACTTAATGTGATTATTTTTTGTCTTCATAGGGTGACTTCAAGTC
TTTTTTAATGTGAACCTTACAGTGGGGTTTGTTCTGAATATATTTATTTG
CATTTGATATAGAGAGGCTCAAAAAAGGAAAACTGACGGACCTAATAAAA
TAAAAAAATAGTAGTTGAATAAAAAAATTTCTGGAGGGACACACAAATAA
TGGTTACTCTCTCTGCATGCCATGGGAGATAGTGGTGAAGGGAGGAGGGA
CACTTTTGCAATTCATTTGATCTCTTCCACAGTATCTGAATTATTTGCAA
GTATACGTATTCCATTTGTAATAAAAAAGTAAAGACTAGAAATTCTTTTT
GGAAAACTGTACATACTGTAGAACATGGCTCTGTGTTGTTTGTCTCTGGA
TTTCCAGTGACTATCAAATAGGTGCTCAAGAAATTGGATAATTATGTGAA
TGAATTCATGAATTGAATGAATGAATGAACAGACTATGCTGTTCTGTTTG
AAAATAAGTATGGACTTACAAGAATGAAAAGTCTTCAACACTTGGAATAT
GTTTTCAATAAAGATCAGGCCTCTCATCTATA.

In some embodiments, the splice variant provided in XR_001746611.1 (ensembl gene id ENSG00000286162.2) is the reference INCA1 sequence:

Homo sapiens uncharacterized LOC107987045, transcript variant X2, ncRNA

| (SEQ ID NO: 2) | |
|---|---|
| 1 | aagcccaaga ggctttccct aactccaaaa cggcgacctt taaagtaagg gagggttggg |
| 61 | gcaccacagc ggccggattt ctaattgtgg aaagtttaaa caaatttgtc cctcggggaa |
| 121 | tgggggaagg gagagaagaa caagtctggg ctcggccggg gagcgtgcgg agtctgcacg |
| 181 | ccgcggccgt cggtccaacg cggggaggct cagggatttt cggcagcagc gacttcatcc |
| 241 | accccggacc gccggctgga ggacgtgcct gcagccggcg ggtacctgcg gccaattcgg |
| 301 | tggaacgagg acgcggactg cgaaggcagg taccctcctc tgaaatcaaa actctttaat |
| 361 | taaagagaca tgaaatgaca agattttaaa aaaatcccat gaggagaagg cattctaatt |
| 421 | taggaagaac tcccccttgg aaaaaccatc agtgccggaa gatttcctat tgtgttgatc |
| 481 | catggcaaag gagactgcag atacacaagg gatattatgg agcccagacg acctgaataa |
| 541 | aacccttccc tactacaagg acagctgtcc cttccctaca cactccctac aggctgatga |
| 601 | gagacctttt ttggaagcag aaacttatac tttatgctgc cttcttcctg actgccagga |
| 661 | ttatactctt cctttccatc ccagatctag caatgctgtt gatgaggcta agtcatgatg |
| 721 | atttctttaa tatcttggaa cacagtagat gcctgatatt tgcggatgga ctggagaaaa |
| 781 | actgaaagta taaaccacaa catctcaaga gatgtcatga atggagaagc atatggtaaa |
| 841 | atataatgaa aattaaatct actttacaag tggtatcttc ttgacaatag tggcattacc |
| 901 | tgagccagta tcagggacca gaaatacaac ataaaactga tacatttctc ttaatcccca |
| 961 | catgattttg tttttgagtt tatagttatc agtgatcatt gattaataat tgatcagtga |
| 1021 | tcagtgacaa atgtatactt tttctacttg tattgttagc cctcagattg atggtagtta |
| 1081 | gcccaggaat ggaaagacca aggggagatg atttaattct agtcaaatat taaaaggtca |
| 1141 | tcatgtaaat gaaataatcc caagactaca actagagggg cagatttcag cttactatta |
| 1201 | gggagtaatt tcaacaggat atggaaggga tatccaaatg gcagggaggc cattgtggga |
| 1261 | tatgacaggg acgccattgc aggaaatata agaacagaag atcctctggg ggatttttgt |
| 1321 | tctttgtggg agtttggatt agctgctcct gcctgggatc ctttcagctc tcaattctgt |
| 1381 | gaaactcaac aatgaaagca gaggtgtttt cctttactta gctagtgcca acacaaaact |
| 1441 | ttgaactcaa gtgacctgat aacataactg tgggtcaata ccgaggaact aacactccta |
| 1501 | gttccttttc aattccccaa gacaacatca gtatagattt tcaagatgaa tatatgccca |
| 1561 | gtaagtatgg cttatcctgt actggaagaa taggttcttt cacacttaat gtgattattt |
| 1621 | tttgtcttca tagggtgact tcaagtcttt tttaatgtga accttacagt ggggtttgtt |
| 1681 | ctgaatatat ttatttgcat ttgatataga gaggctcaaa aaaggaaaac tgacggacct |
| 1741 | aataaaataa aaaaatagta gttgaataaa aaaatttctg gagggacaca caaataatgg |
| 1801 | ttactctctc tgcatgccat gggagatagt ggtgaaggga ggagggacac ttttgcaatt |
| 1861 | catttgatct cttccacagt atctgaatta tttgcaagta tacgtattcc atttgtaata |
| 1921 | aaaaagtaaa gactagaaat tctttttgga aaactgtaca tactgtagaa catggctctg |
| 1981 | tgttgtttgt ctctggattt ccagtgacta tcaaataggt gctcaagaaa ttggataatt |
| 2041 | atgtgaatga attcatgaat tgaatgaatg aatgaacaga ctatgctgtt ctgtttgaaa |
| 2101 | ataagtatgg acttacaaga atgaaaagtc ttcaacactt ggaatatgtt ttcaataaag |
| 2161 | atcaggcctc tcatctataa taat |

Exemplary sequences for HNRNPH1 are available in genbank, including NM_001257293.2 (nucleic acid, variant 1) and NP_001244222.1 (protein: heterogeneous nuclear ribonucleoprotein H isoform a). This variant 1 represents the longest transcript and encodes the longer isoform a. An exemplary sequence is as follows:

| (SEQ ID NO: 53) | |
|---|---|
| 1 | catttcgtct tagccacgca gaagtcgcgt gtctaggtga gtcgcggtgg gtcctcgctt |
| 61 | gcagttcagc gaccacgttt gtttcgacgc cggaccgcgt aagagacgat gatgttgggc |
| 121 | acggaaggtg gagagggatt cgtggtgaag gtccggggct tgccctggtc ttgctcggcc |
| 181 | gatgaagtgc agaggttttt ttctgactgc aaaattcaaa atggggctca aggtattcgt |
| 241 | ttcatctaca ccagagaagg cagaccaagt ggcgaggctt ttgttgaact tgaatcagaa |
| 301 | gatgaagtca aattggccct gaaaaaagac agagaaacta tgggacacag atatgttgaa |
| 361 | gtattcaagt caaacaacgt tgaaatggat tgggtgttga agcatactgg tccaaatagt |
| 421 | cctgacacgg ccaatgatgg ctttgtacgg cttagaggac ttccctttgg atgtagcaag |
| 481 | gaagaaattg ttcagttctt ctcagggttg gaaatcgtgc caaatgggat aacattgccg |
| 541 | gtggacttcc aggggaggag tacgggggag gccttcgtgc agtttgcttc acaggaaata |
| 601 | gctgaaaagg ctctaaagaa acacaaggaa agaatagggc acaggtatat tgaaatcttt |
| 661 | aagagcagta gagctgaagt tagaactcat tatgatccac cacgaaagct tatggccatg |
| 721 | cagcggccag gtccttatga cagacctggg gctggtagag ggtataacag cattggcaga |
| 781 | ggagctggct ttgagaggat gaggcgtggt gcttatggtg gaggctatgg aggctatgat |
| 841 | gattacaatg gctataatga tggctatgga tttgggtcag atagatttgg aagagacctc |
| 901 | aattactgtt tttcaggaat gtctgatcac agatacgggg atggtggctc tactttccag |
| 961 | agcacaacag gacactgtgt acacatgcgg ggattacctt acagagctac tgagaatgac |
| 1021 | atttataatt ttttttcacc gctcaaccct gtgagagtac acattgaaat tggtcctgat |
| 1081 | ggcagagtaa ctggtgaagc agatgtcgag ttcgcaactc atgaagatgc tgtggcagct |
| 1141 | atgtcaaaag acaaagcaaa tatgcaacac agatatgtag aactcttctt gaattctaca |
| 1201 | gcaggagcaa gcggtggtgc ttacgaacac agatatgtag aactcttctt gaattctaca |
| 1261 | gcaggagcaa gcggtggtgc ttatggtagc caaatgatgg gaggcatggg cttgtcaaac |
| 1321 | cagtccagct acgggggccc agccagccag cagctgagtg ggggttacgg aggcggctac |
| 1381 | ggtggccaga gcagcatgag tggatacgac caagttttac aggaaaactc cagtgatttt |
| 1441 | caatcaaaca ttgcataggt aaccaaggag cagtgaacag cagctactac agtagtggaa |
| 1501 | gccgtgcatc tatgggcgtg aacggaatgg gagggttgtc tagcatgtcc agtatgagtg |
| 1561 | gtggatgggg aatgtaattg atcgatcctg atcactgact cttggtcaac cttttttttt |
| 1621 | tttttttttt tttctttaag aaaacttcag tttaacagtt tctgcaatac aagcttgtga |
| 1681 | tttatgctta ctctaagtgg aaatcaggat tgttatgaag acttaaggcc cagtattttt |
| 1741 | gaatacaata ctcatctagg atgtaacagt gaagctgagt aaactataac tgttaaactt |
| 1801 | aagttccagc ttttctcaag ttagttatag gatgtactta agcagtaagc gtatttaggt |
| 1861 | aaaagcagtt gaattatgtt aaatgttgcc ctttgccacg ttaaattgaa cactgttttg |
| 1921 | gatgcatgtt gaaagacatg cttttatttt tttgtaaaac aatataggag ctgtgtctac |
| 1981 | tattaaaagt gaaacatttt ggcatgtttg ttaattctag tttcatttaa taacctgtaa |
| 2041 | ggcacgtaag tttaagcttt tttttttttt aagttaatgg gaaaaatttg agacgcaata |
| 2101 | ccaatactta ggattttggt cttggtgttt gtatgaaatt ctgaggcctt gatttaaatc |
| 2161 | tttcattgta ttgtgatttc cttttaggta tattgcgcta agtgaaactt gtcaaataaa |
| 2221 | tcctcctttt aaaaactgca |

Other variants of HNRNPH1 can also be targeted. The gene sequence can be found in NCBI at NC_000005.10 (Reference GRCh38.p13 Primary Assembly), Range 179614178-179634784 (complement).

Inhibitory nucleic acids targeting INCA or hnRPH1 useful in the present methods and compositions include antisense oligonucleotides, ribozymes, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics that hybridize to at least a portion of the target INCA or hnRPH1 lncRNA and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19,20,21,22,23,24,25,26,27,28,29,30,31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this disclosure, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

Antisense

In some embodiments, the inhibitory nucleic acids are antisense oligonucleotides. Antisense oligonucleotides are typically designed to block expression of a DNA or RNA target by binding to the target and halting expression at the level of transcription, translation, or splicing. Antisense oligonucleotides of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to give the desired effect.

siRNA/shRNA

In some embodiments, the nucleic acid sequence that is complementary to a target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002).

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261: 1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. In some embodiments, the oligonucleotide is a gapmer (contain a central stretch (gap) of DNA monomers sufficiently long to induce RNase H cleavage, flanked by blocks of LNA modified nucleotides; see, e.g., Stanton et al., Nucleic Acid Ther. 2012. 22: 344-359; Nowotny et al., Cell, 121:1005-1016, 2005; Kurreck, European Journal of Biochemistry 270:1628-1644, 2003; Fluiter et al., Mol Biosyst. 5(8):838-43, 2009). In some embodiments, the oligonucleotide is a mixmer (includes alternating short stretches of LNA and DNA; Naguibneva et al., Biomed Pharmacother. 2006 November; 60(9):633-8; Ørom et al., Gene. 2006 May 10; 372:137-41). Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NHO—CH₂, CH, —N(CH₃)—O—CH₂ (known as a methylene(methylimino) or MMI backbone], CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH; amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃OCH₃, OCH₃O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O-, S-, or N-alkenyl; SOCH3; SO2 CH3; ONO2; NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given oligonucleotide to be uniformly modified, and in fact more than one of the aforementioned modifications may be incorporated in a single oligonucleotide or even at within a single nucleoside within an oligonucleotide.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition’, 1991, 30, page 613, and those disclosed by Sanghvi, Y. S., Chapter 15, Antisense Research and Applications', pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the oligonucleotide. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxygen and the 4′-carbon—i.e., oligonucleotides containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of oligonucleotides of 10-30 nucleotides spanning the length of a target RNA can be prepared, followed by testing for activity. Optionally, gaps, e.g., of 5-10 nucleotides or more, can be left between the LNAs to reduce the number of oligonucleotides synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) oligonucleotides). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2-modified nucleotide, e.g., a 2′-deoxy, 2′-deoxy-2′-fluoro, 2-O-methyl, 2′-0-methoxyethyl (2-O-MOE), 2′-O-aminopropyl (2-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-0 atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I. Theory and Nucleic Acid Preparation, Tijssen, ed. Elsevier, N.Y. (1993).

Pharmaceutical Compositions

The methods described herein can include the administration of pharmaceutical compositions and formulations comprising inhibitory nucleic acid sequences designed to target an RNA.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Krützfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

Immunotherapy

The methods can also include administering an immunotherapy, e.g., an immune checkpoint inhibitor; cancer vaccines; dendritic cell vaccine; adaptive T cell therapy; and/or chimeric antigen receptor-expressing immune effector cells, e.g., CAR-T cells. In preferred embodiments, the immunotherapy results in an increase in IFNγ activity and/or levels.

Currently approved immune checkpoint inhibitors include monoclonal antibodies (mAbs) that target the programmed cell death protein 1 (PD-1)/PD-L1/2 or cytotoxic T-lymphocyte-associated protein 4 (CTLA-4) pathways, and agents targeting other pathways are in clinical development (including OX40, Tim-3, and LAG-3) (See, e.g., Leach et al., Science 271, 1734-1736 (1996); Pardoll, Nat. Rev. Cancer 12, 252-264 (2012); Topalian et al., Cancer Cell 27, 450-461 (2015); Mahoney et al., Nat Rev Drug Discov 14, 561-584 (2015)). The present methods can include the administration of checkpoint inhibitors such as antibodies including anti-CD137 (BMS-663513); anti-PD-1 (programmed cell death 1) antibodies (including those described in U.S. Pat. Nos. 8,008,449; 9,073,994; and US20110271358, pembrolizumab, nivolumab, Pidilizumab (CT-011), BGB-A317, MEDI0680, BMS-936558 (ONO-4538)); anti-PDL1 (programmed cell death ligand 1) or anti-PDL2 (e.g., BMS-936559, MPDL3280A, atezolizumab, avelumab and durvalumab); or anti-CTLA-4 (e.g., ipilumimab or tremelimumab). See, e.g., Krüger et al., “Immune based therapies in cancer,” Histol Histopathol. 2007 June; 22(6):687-96; Eggermont et al., “Anti-CTLA-4 antibody adjuvant therapy in melanoma,” Semin Oncol. 2010 October; 37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells:

Alternatively or in addition, the immunotherapy can include administration of a population of immune effector cells (e.g., T cells or Natural Killer (NK) cells) that can be engineered to express one or more Chimeric Antigen Receptors (CARs). CARs are hybrid molecules comprising three essential units: (1) an extracellular antigen-binding motif, (2) linking/transmembrane motifs, and (3) intracellular T-cell signaling motifs (Long A H, Haso W M, Orentas R J. Lessons learned from a highly-active CD22-specific chimeric antigen receptor. Oncoimmunology. 2013; 2 (4):e23621). Such T cells are referred to as CAR-T cells. See, e.g., US20180355052A1; WO2017117112A1; US20190309307; US20190292246; US 20190298772; US20190000880; US20150376296; Bollino and Webb, “Chimeric antigen receptor-engineered natural killer and natural killer T cells for cancer.” Immunotherapy. Translational Research 2017; 187:32-43; Fu and Tang, Recent patents on Anti-Cancer Drug Discovery, 14(1), 2019; 14(1):60-69, DOI: 10.2174/1574892814666190111120908; Jürgens and Clarke, Nature Biotechnology 37:370-375 (2019); and references cited therein. In some embodiments, the CAR-T cells are autologous, i.e., derived from the same individual to whom it is later to be re-introduced e.g., during therapy. In some embodiments, the CAR-T cells are non-autologous, i.e., derived from a different individual relative to the individual to whom the material is to be introduced. Alternatively, a T cell-engaging therapeutic agent, such as a bispecific or other multispecific agent, e.g., antibody that is capable of recruiting and/or engaging the activity of one or more T cells, such as in a target-specific manner, can be used (see US 20190292246).

A number of immunotherapies are known in the art. In some embodiments, these therapies may primarily target immunoregulatory cell types such as regulatory T cells (Tregs) or M2 polarized macrophages, e.g., by reducing number, altering function, or preventing tumor localization of the immunoregulatory cell types. For example, Treg-targeted therapy includes anti-GITR monoclonal antibody (TRX518), cyclophosphamide (e.g., metronomic doses), arsenic trioxide, paclitaxel, sunitinib, oxaliplatin, PLX4720, anthracycline-based chemotherapy, Daclizumab (anti-CD25); Immunotoxin eg. Ontak (denileukin diftitox); lymphoablation (e.g., chemical or radiation lymphoablation) and agents that selectively target the VEGF-VEGFR signaling axis, such as VEGF blocking antibodies (e.g., bevacizumab), or inhibitors of VEGFR tyrosine kinase activity (e.g., lenvatinib) or ATP hydrolysis (e.g., using ectonucleotidase inhibitors, e.g., ARL67156 (6-N,N-Diethyl-D-β,γ-dibromomethyleneATP trisodium salt), 8-(4-chlorophenylthio) cAMP (pCPT-cAMP) and a related cyclic nucleotide analog (8-[4-chlorophenylthio] cGMP; pCPT-cGMP) and those described in WO 2007135195, as well as mAbs against CD73 or CD39). Docetaxel also has effects on M2 macrophages. See, e.g., Zitvogel et al., Immunity 39:74-88 (2013). In another example, M2 macrophage targeted therapy includes clodronate-liposomes (Zeisberger, et al., Br J Cancer. 95:272-281 (2006)), DNA based vaccines (Luo, et al., J Clin Invest. 116(8): 2132-2141 (2006)), and M2 macrophage targeted pro-apoptotic peptides (Cieslewicz, et al., PNAS. 110(40): 15919-15924 (2013)). Immnotherapies that target Natural Killer T (NKT) cells can also be used, e.g., that support type I NKT over type II NKT (e.g., CD1d type I agonist ligands) or that inhibit the immune-suppressive functions of NKT, e.g., that antagonize TGF-beta or neutralize CD1d.

Some useful immunotherapies target the metabolic processes of immunity, and include adenosine receptor antagonists and small molecule inhibitors, e.g., istradefylline (KW-6002) and SCH-58261; indoleamine 2,3-dioxygenase (IDO) inhibitors, e.g., Small molecule inhibitors (e.g., 1-methyl-tryptophan (1MT), 1-methyl-d-tryptophan (D1MT), and Toho-1) or IDO-specific siRNAs, or natural products (e.g., Brassinin or exiguamine) (see, e.g., Munn, Front Biosci (Elite Ed). 2012 Jan. 1; 4:734-45) or monoclonal antibodies that neutralize the metabolites of IDO, e.g., mAbs against N-formyl-kynurenine.

In some embodiments, the immunotherapies may antagonize the action of cytokines and chemokines such as IL-10, TGF-beta, IL-6, CCL2 and others that are associated with immunosuppression in cancer. For example, TGF-beta neutralizing therapies include anti-TGF-beta antibodies (e.g. fresolimumab, Infliximab, Lerdelimumab, GC-1008), antisense oligodeoxynucleotides (e.g., Trabedersen), and small molecule inhibitors of TGF-beta (e.g. LY2157299), (Wojtowicz-Praga, Invest New Drugs. 21(1): 21-32 (2003)). Another example of therapies that antagonize immunosuppression cytokines can include anti-IL-6 antibodies (e.g. siltuximab) (Guo, et al., Cancer Treat Rev. 38(7):904-910 (2012). mAbs against IL-10 or its receptor can also be used, e.g., humanized versions of those described in Llorente et al., Arthritis & Rheumatism, 43(8): 1790-1800, 2000 (anti-IL-10 mAb), or Newton et al., Clin Exp Immunol. 2014 July; 177(1):261-8 (Anti-interleukin-10R1 monoclonal antibody). mAbs against CCL2 or its receptors can also be used. In some embodiments, the cytokine immunotherapy is combined with a commonly used chemotherapeutic agent (e.g., gemcitabine, docetaxel, cisplatin, tamoxifen) as described in U.S. Pat. No. 8,476,246.

In some embodiments, immunotherapies can include agents that are believed to elicit “danger” signals, e.g., “PAMPs” (pathogen-associated molecular patterns) or “DAMPs” (damage-associated molecular patterns) that stimulate an immune response against the cancer. See, e.g., Pradeu and Cooper, Front Immunol. 2012, 3:287; Escamilla-Tilch et al., Immunol Cell Biol. 2013 November-December; 91(10):601-10. In some embodiments, immunotherapies can agonize toll-like receptors (TLRs) to stimulate an immune response. For example, TLR agonists include vaccine adjuvants (e.g., 3M-052) and small molecules (e.g., Imiquimod, muramyl dipeptide, CpG, and mifamurtide (muramyl tripeptide)) as well as polysaccharide krestin and endotoxin. See, Galluzi et al., Oncoimmunol. 1(5): 699-716 (2012), Lu et al., Clin Cancer Res. Jan. 1, 2011; 17(1): 67-76, U.S. Pat. Nos. 8,795,678 and 8,790,655. In some embodiments, immunotherapies can involve administration of cytokines that elicit an anti-cancer immune response, see Lee & Margolin, Cancers. 3: 3856-3893(2011). For example, the cytokine IL-12 can be administered (Portielje, et al., Cancer Immunol Immunother. 52: 133-144 (2003)) or as gene therapy (Melero, et al., Trends Immunol. 22(3): 113-115 (2001)). In another example, interferons (IFNs), e.g., IFNgamma, can be administered as adjuvant therapy (Dunn et al., Nat Rev Immunol. 6: 836-848 (2006)).

In some embodiments, immunotherapies can antagonize cell surface receptors to enhance the anti-cancer immune response. For example, antagonistic monoclonal antibodies that boost the anti-cancer immune response can include antibodies that target CTLA-4 (ipilimumab, see Tarhini and Iqbal, Onco Targets Ther. 3:15-25 (2010) and U.S. Pat. No. 7,741,345 or Tremelimumab) or antibodies that target PD-1 (nivolumab, see Topalian, et al., NEJM. 366(26): 2443-2454 (2012) and WO2013/173223A1, pembrolizumab/MK-3475, Pidilizumab (CT-011)).

Some immunotherapies enhance T cell recruitment to the tumor site (such as Endothelin receptor-A/B (ETRA/B) blockade, e.g., with macitentan or the combination of the ETRA and ETRB antagonists BQ123 and BQ788, see Coffman et al., Cancer Biol Ther. 2013 February; 14(2):184-92), or enhance CD8 T-cell memory cell formation (e.g., using rapamycin and metformin, see, e.g., Pearce et al., Nature. 2009 Jul. 2; 460(7251):103-7; Mineharu et al., Mol Cancer Ther. 2014 Sep. 25. pii: molcanther.0400.2014; and Berezhnoy et al., Oncoimmunology. 2014 May 14; 3:e28811). Immunotherapies can also include administering one or more of: adoptive cell transfer (ACT) involving transfer of ex vivo expanded autologous or allogeneic tumor-reactive lymphocytes, e.g., dendritic cells or peptides with adjuvant; cancer vaccines such as DNA-based vaccines, cytokines (e.g., IL-2), cyclophosphamide, anti-interleukin-2R immunotoxins, Prostaglandin E2 Inhibitors (e.g., using SC-50) and/or checkpoint inhibitors including antibodies such as anti-CD137 (BMS-663513), anti-PD1 (e.g., Nivolumab, pembrolizumab/MK-3475, Pidilizumab (CT-011)), anti-PDL1 (e.g., BMS-936559, MPDL3280A), or anti-CTLA-4 (e.g., ipilumimab; see, e.g., Krüger et al., “Immune based therapies in cancer,” Histol Histopathol. 2007 June; 22(6):687-96; Eggermont et al., “Anti-CTLA-4 antibody adjuvant therapy in melanoma,” Semin Oncol. 2010 October; 37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells: overcoming suppression of T-cell immunity,” Cancer J. 2010 July-August; 16(4):342-7. In some embodiments, the methods include administering a composition comprising tumor-pulsed dendritic cells, e.g., as described in WO2009/114547 and references cited therein. See also Shiao et al., Genes & Dev. 2011. 25: 2559-2572.

For further information regarding immunotherapies that can be used in the present methods, see Christofi et al., Cancers (Basel). 2019 Sep. 30; 11(10). pii: E1472; Demaria et al., Nature. 2019 October; 574(7776):45-56; and Bastien et al., Seminars in Immunology 42 (2019) 101306, and references cited therein.

### EXAMPLES

The invention is further described in the following examples, which do not limit the scope of the invention described in the claims.

Materials and Methods

The following materials and methods were used in the Examples below.

Cell Lines

Patient-derived primary GBM cells (PDGCLs, BT cell lines) were generated as previously described (25). U251 cells were purchased from the American Type Culture Collection (ATCC). U1242 cells were obtained from James Van Brocklyn (Ohio State University). A375 cells were obtained from Frank Stephen Hodi (Dana-Farber Cancer Institute). H2122 and H1703 cells were obtained from Sandro Santagata (Brigham and Women's Hospital). MDA-MB-231 cells were obtained from David Walt (Brigham and Women's Hospital). BT cell lines were cultured as neurospheres in stem cell conditions using Neurobasal (Thermo Fisher Scientific) supplemented with Glutamine (Thermo Fisher Scientific), B27 (Thermo Fisher Scientific), 20 ng/ml epidermal growth factor (EGF) and fibroblast growth factor (FGF)-2 (PrepoTech). U251, U1242, A375, H2122 and H1703 cells were cultured in DMEM (Thermo Fisher Scientific) supplemented with 10% fetal bovine serum (FBS, Sigma-Aldrich) and 100 U/ml penicillin-streptomycin (Thermo Fisher Scientific). MDA-MB-231 cells were cultured in RPMI (Thermo Fisher Scientific) supplemented with 10% FBS and 100 U/ml penicillin-streptomycin. Unless otherwise specified, IFNγ stimulation was performed at 100 U/ml IFNγ for a period of 24 h. GapmeRs (Exiqon) knockdown experiments were performed transfecting BT cells with 50 nM of GapmeR 1 (ATTAGGTCCGTCAGT (SEQ ID NO:3)) or GapmeR 2 (TTACATGATGACCTTT (SEQ ID NO:4)) using Lipofectamine 2000 (Thermo Fisher Scientific). Stable U251, A375 and MDA-MB-231 knockdown were obtained by infecting cells with shRNA 1 (clone: CS-SH128T-3-LVRU6GP; target sequence: GCCATTGCAGGAAATATAAGA (SEQ ID NO:5), GeneCopoeia) and shRNA 2 (clone: CS-SH128T-6-LVRU6GP; target sequence: CAGCTCTCAATTCTGTGAAACTCAA (SEQ ID NO:6), GeneCopoeia). Stable U251-EGFRvIII were obtained by infecting cells with pLV-IRES-mCherry-EGFRvIII vector. HNRNPH1 knockdown was performed transfecting 50 pmol/well of Duplex siRNAs (hs.Ri.HNRNPH1.13.1 and hs.Ri.HNRNPH1.13.2, Integrated DNA Technologies) for 6 well plate using Lipofectamine RNAiMAX (Thermo Fisher Scientific). HNRNPH1 binding site blocking experiments were performed transfecting cells with 100 nM of fully 2′-O-Methoxyethyl (2′-MOE) and phosphorothioate bond modified antisense oligonucleotide control (ASO NC, GCGACTATACGCGCAATATG (SEQ ID NO:7)) or targeting HNRNPH1 binding site on the INCA1 gene (ASO H1B, CTCCAGCTCCCCCCGGCAAC (SEQ ID NO:8)) (Integrated DNA Technologies).

Human Specimens

Tumor tissue samples were obtained as approved by the Institutional Review Board (IRB) at the Dana-Farber Cancer Institute. Patient samples were processed for extraction of total RNA.

RNA-Seq and Analysis of RNA-Seq Data

BT333, U251 shControl and U251 shINCA1 cells were stimulated with IFNγ for 24 h. RNA was extracted using RNeasy kit (QIAGEN). 1p g of total RNA from unstimulated and IFNγ-stimulated cells was used and ribosomal RNA was removed using Ribo-Zero rRNA Removal kit (Illumina). RNA libraries were prepared using NEBNext Ultra II RNA Library Prep Kit for Illumina (New England BioLabs). Paired-end reads were sequences on a HiSeq System (Illumina) to achieve at least 40 million reads per sample. Libraries prepared from three independent experiments were analyzed. Raw reads were examined for quality issues using FastQC. Trimmed reads were aligned to the UCSC build 38 of the human genome (h38), augmented with transcript information from Ensembl release GRCh38 using STAR. Count of reads aligning known genes was generated by HT-Seq. Differential expression at the gene level was calculated with DESeq. To identify lncRNAs associated with INCA1 expression, we used PARIS algorithm, a Genepattern module that uses a mutual information-based metric (RNMI) to rank genetic features based on the degree of correlation to the target profile (INCA1).

Determination of Somatic Copy Number Alterations

Somatic copy-number alterations (SCNAs) were determined from whole-exome sequencing data (PDGCLs) and SNP 6.0 Affymetrix microarray data (LTGCLs and TCGA samples) using Genomic Identification of Significant Targets in Cancer (GISTIC 2.0) (26). SNP array data from the LTGCLs and 187 normal control samples were downloaded from the CCLE data portal (broadinstitute.org/ccle/data) on 29 Sep. 2012 and analyzed using the GenePattern Copy Number Inference pipeline (27) to generate raw copy-number estimates. These estimates were further refined using Tangent normalization (27) against 3,000 normal samples profiled by TCGA. Copy-number calls were made using circular binary segmentation (CBS) (28). Copy-number calls for TCGA samples were generated as previously described (29, 30). Copy-number calls were made from PDGCLs using ReCapSeg (gatkforums.broadinstitute.org/gatk/categories/recapseg-documentation). For cell lines (PDGCLs and LTGCLs), GISTIC 2.0 algorithm was calculated at gene level using next parameters: Amplification/Deletion threshold=0.3, joint segment size=10, Significance threshold (q-value)=0.25. For each gene, GISTIC provided the following SCNA calls: High Level Gain (Amplifications), Low Level Gains, Low Level Loss, and High Level Loss (Homozygous Deletions). Amplifications and Low Level Gains represent copy-number log 2ratios of >0.9 and between 0.9 and 0.3, respectively; Homozygous Deletions and Low Level Losses represent copy-number ratios of <−1.3 and between −1.3 and 0.3, respectively.

Quantitative Real-Time PCR Analysis

Total RNA from cell lines and patients' tissues was extracted using TRIzol (Thermo Fisher Scientific). Nuclear/cytoplasmic fractionation was performed as previously described (13). RNA was reverse-transcribed using iScript cDNA Synthesis Kit (BioRad) and quantitative real-time PCR was performed using SYBR Green Master Mix (Applied Biosystem). 18S expression levels were used as control. For copy number analysis, absolute quantification of INCA1 and PD-L1 RNA was performed using the standard-curve method. The primers in the following table were used throughout the study.

| | SEQ |
|---|---|
| Name: Sequence | ID NO: |
| 18S For: AACTTTCGATGGTAGTCGCCG | 9 |
| 18S Rev: CCTTGGATGTGGTAGCCGTTT | 10 |
| INCA1 For: GTGGGATATGACAGGGACGC | 11 |
| INCA1 Rev: GGCAGGAGCAGCTAATCCAA | 12 |
| INCA1 Full-length For: | 13 |
| AAGCCCAAGAGGCTTTCCCT | |
| INCA1 Full-length Rev: | 14 |
| ATTATTATAGATGAGAGGCCTGATC | |
| PD-L1 For: GAGTGGTAAGACCACCACC | 15 |
| PD-L1 Rev: GGTTTTCCTCAGGATCTAAT | 16 |
| PD-L2 For: AGTGCTATCTGAACCTGTGGTC | 17 |
| PD-L2 Rev: AGTGCTGGGTCATCCAAAGG | 18 |
| RIC1 For: CTCCGCACGGACCATGTATT | 19 |
| RIC1 Rev: TCGGACTGAACGTGGAAAGG | 20 |
| HNRNPH1 For: CCAGAGCAGCATGAGTGGAT | 21 |
| HNRNPH1 Rev: TAGCTGCTGTTCACTGCTCC | 22 |
| MALAT1 For: AGCAGACACACGTATGCGAA | 23 |
| MALAT1 Rev: GTGGTTCCCAATCCCCACAT | 24 |
| NORAD For: GTCCTGACGACAACGGACAA | 25 |
| NORAD Rev: AGAATGAAGACCAACCGCCC | 26 |
| RMRP For: CACGTAGACATTCCCCGCTT | 27 |
| RMRP Rev: CTGCCTGCGTAACTAGAGGG | 28 |

Immunoblot Analysis and Antibodies

Immunoblotting was performed as previously described (13). The following antibodies were used: anti-PD-L1, anti-IDO and anti-β-Actin (13684, 86630 and 3700, respectively, Cell Signaling Technology); anti-hnRNP-H (A300-511A, Bethyl Laboratories).

In Vitro Transcription/Translation Assay

In vitro transcription and translation was performed using TnT Quick Coupled Transcription/Translation System and Transcend Non-Radioactive Translation Detection System (Promega) following the manufacturer's instructions. The lncRNA was transcribed from a T7 promoter of a pcDNA3.1 plasmid (Thermo Fisher Scientific). The pSP64-Luciferase plasmid supplied with the TnT Quick Coupled Transcription/Translation System and a GFP cloned in pcDNA3.1 plasmid were used as a positive control.

CD8⁺ T cell isolation

Peripheral blood mononuclear cells (PBMCs) were obtained from healthy human donors as approved by the IRB at the Brigham and Women's Hospital. PBMCs were isolated using Ficoll Paque Plus (GE Healthcare Life Sciences) following the manufacturer's instructions. CD8⁺ T cells were isolated by negative selection using the CD8⁺ T Cell Isolation Kit (Miltenyi Biotec). Isolation was performed according to the manufacturer's recommendations.

Flow Cytometry

Cells were harvested at the indicated time-points and washed with FACS buffer (PBS supplemented with 2% FBS). Cells were stained incubating with the indicated antibodies diluted in FACS buffer. After staining cells were fixed 2% paraformaldehyde (PFA). Flow cytometry was performed on a BD LSR II (BD Biosciences) and data analyzed using FlowJo. The following antibodies were used: anti-CD274 (PD-L1, 329706, BioLegend); LIVE/DEAD Fixable Near-IR Dead Cell Stain Kit (L10119, Thermo Fisher Scientific).

ELISA Assay

Control and INCA1 knockdown tumor cells were co-cultured at a 1:1 ratio with non-stimulated CD8⁺ T cells or CD8⁺ T cells stimulated with Dynabeads Human T-Activator CD3/CD28 (Thermo Fisher Scientific). Culture media was collected and secreted levels of IFNγ were analyzed at 48 h using Human IFNγ ELISA MAX Deluxe (BioLegend). The absorbance was read at 450 nm using a microplate reader. Secreted IFNγ was quantified based on the standard curve

T Cell Cytotoxicity Assay

For 2D assays, control or INCA1 knockdown tumor cells were co-cultured for 96 h at a 1:1 ratio with non-stimulated CD8⁺ T cells or CD8⁺ T cells stimulated with Dynabeads Human T-Activator CD3/CD28 (Thermo Fisher Scientific) and 10 ng/ml interleukin-2 (PeproTech). T cells were then removed washing with PBS. Cell viability was determined using CellTiter-Glo Luminescent Cell Viability Assay (Promega). Alternatively, after incubation, tumor cells were harvested and stained using LIVE/DEAD staining. Percent of dead cells was determined by flow cytometry. For 3D assays, 750 GFP positive control or INCA1 knockdown tumor cells were seeded in a round bottom low-attachment 96 well plate. Cells were allowed to form tumorspheres for 72 h. After tumorspheres were formed, two thousand non-stimulated CD8⁺ T cells or CD8⁺ T cells stimulated with Dynabeads Human T-Activator CD3/CD28 and 10 ng/ml interleukin-2 were added. Tumorspheres and CD8⁺ T cells were co-cultured for 96 h and changes in GFP intensity were measured using ImageJ.

In Vitro T Cells Transduction

Generation of T cells expressing chimeric antigen receptor (CAR) against EGFRvIII was constructed as described previously (31) using the self-inactivating lentiviral transfer vector pRRL.PPT.EFS bearing an IRS-GFP cassette and packaged as described previously (32). pRRL.PPT.EFS-GFP vector served as control. T cells were isolated from PBMCs by EasySep Human T cell isolation kit (Stem Cell Technology). Isolated T cells were counted and cultured at 1:1 ratio with Dynabeads human T activator CD3/CD28 (Thermo Fisher Scientific) in X-vivo15 medium supplemented with 30U/ml IL-2. Next day, 1.5 million T cells/ml were transduced with EGFRvIII-CAR or control lentivirus at MOI 10 and 6 ug/ml polybrene in 6 well plates. Medium was replaced next morning and GFP expression was checked 48 hours post-infection. Before injecting T cells in mice, the ability of EGFRvIII-specific CAR T cells to kill target cells was tested in vitro by 3D T cell cytotoxicity assay.

In Vivo Studies

Mice studies were conducted according to the protocols approved by the Institutional Animal Care and Use Committee (IACUC). 2.5×10⁶ U251 EGFRvIII shControl or shINCA1 were injected subcutaneously into 6- to 8-week-old male and female NSG mice (Jackson), with 6 mice per group (n=2 males and n=4 females). Seven days after tumor implantation, 1×10⁶ CAR T cells or GFP transduced T cells were injected intravenously via tail vein in 200 μl of PBS. Tumor size was measured by calipers in three dimensions, L×W×H, for the duration of the experiments. Tumor growth was followed for 4 weeks, or until predetermined IACUC-approved endpoint was reached.

RNA Antisense Purification (RAP)

RAP was performed as previously described with some modifications (33). For RAP-MS (Mass Spectrometry), BT cells were stimulated with IFNγ and nuclear extracts incubated with biotinylated probes against INCA1 at 67° C. for 2 h. Scrambled biotinylated probe was used as control. RNA was purified using streptavidin agarose beads (Thermo Fisher Scientific). Co-purified proteins were analyzed by Mass Spectrometry as previously described (13). For RAP-RNA, proteins were digested using Proteinase K (Thermo Fisher Scientific) and RNA was extracted using TRIzol.

UV-Crosslink RNA Immunoprecipitation

UV-crosslink RNA immunoprecipitation assay was performed as previously described with some modifications (13). Briefly, cells were UV irradiated at 400 mJ/cm² and nuclear extracts were prepared by incubating cells in RLN Buffer (50 mM Tris, 1.5 mM MgCl₂, 150 mM NaCl, 0.5% NP-40, protease inhibitors) for 5 min. Nuclei were pelleted by centrifuging at 1450×g for 2 min and lysed for 10 min in CLIP Buffer (50 mM Tris, 150 mM NaCl, 1% NP-40, 0.1% Sodium Deoxycholate, phosphatase and protease inhibitors, 100 U/ml RNase inhibitor [New England BioLabs]). Samples were sonicated with microtip, 5 watts power (25% duty) for 60 seconds total in pulses of 1 second on followed by 3 seconds off. DNA was digested incubating samples for 15 min at 37° C. in 1× DNase salt solution (2.5 mM MgCl₂, 0.5 mM CaCl₂) with 30 U TurboDNase. EDTA was added to the samples to a final concentration of 4 mM and samples centrifuged at 16,000×g for 10 min. Nuclear extracts were precleared with Protein A/G Plus Agarose beads (Thermo Fisher Scientific) and incubated with primary antibody (anti-hnRNP-H) or rabbit IgG control (Bethyl Laboratories) overnight at 4° C. Protein/RNA complexes were precipitated using Protein A/G Plus Agarose beads. Beads were washed and incubated with Proteinase K (Thermo Fisher Scientific) and RNA was extracted using TRIzol.

Enhanced CLIP (eCLIP)

A375 cells were stimulated with IFNγ for 6 h and UV irradiated at 400 mJ/cm². eCLIP was performed as previously described (34).

Expression and Purification of HNRNPH1

HNRNPH1 isoform A was cloned in pET21-His-Smt3 and protein expressed by transformation of Rosetta-2 (DE3) pLys(S) E. coli (EMD Millipore). Cells were lysed in 20 mL of 50 mM Tris [pH 8.0], 300 mM KCl, 0.02% NP-40, 10 mM Imidazol, 10% Glycerol, 0.1 mM EDTA, 0.1 mM DTT, 0.1 μg/μl lysozyme. Resuspended cells were incubated on ice for 20 min. Cells were further disrupted and DNA was sheared by sonication (3, 20 second bursts with 20 seconds rests). Insoluble material was pelleted by centrifugation (30 min at 20,000×g at 4° C.). Soluble material was decanted. Insoluble pellet was resolubilized in 50 mM Tris [pH 8.0], 300 mM KCl, 0.02% NP-40, 10 mM Imidazol, 10% Glycerol, 0.1 mM EDTA 0.1 mM DTT, 6 M Urea followed by sonication. Remaining insoluble material was pelleted by centrifugation (30 min at 20,000×g at 4° C.). Soluble material was decanted to new tube. Expression was analyzed by Coomassie staining. 1 mL of TALON resin (Clontech) was equilibrated in respective lysis buffers and added to lysates. Beads and lysates were tumbled at 4° C. for 2 hours. Beads were washed 2 times in 50 mL lysis buffer and loaded onto column. Column was washed with 10 mL of lysis buffer and then eluted in Lysis buffer containing 300 mM Imidazole. 10 fractions of 1.5 mL were collected and analyzed by Coomassie staining. Protein purified under denaturing conditions was dialyzed against 50 mM Tris [pH 8.0], 300 mM KCl, 0.02% NP-40, 10 mM Imidazole, 10% Glycerol, 0.1 mM EDTA 0.1 mM DTT, 4 M Urea overnight. The following day, protein was dialyzed against 50 mM Tris [pH 8.0], 300 mM KCl, 0.02% NP-40, 10 mM Imidazole, 10% Glycerol, 0.1 mM EDTA 0.1 mM DTT, 2 M Urea for 4 hours and then 50 mM Tris pH 8.0, 300 mM KCl, 0.02% NP-40, 10 mM Imidazole, 10% Glycerol, 0.1 mM EDTA 0.1 mM DTT, 1 M Urea for 4 hours. Finally, the protein was dialyzed against 50 mM Tris [pH 8.0], 300 mM KCl, 0.02% NP-40, 10% Glycerol, 0.1 mM EDTA 0.1 mM DTT, 0 M Urea overnight. Dialyzed protein was clarified by centrifugation (30 min at 20,000×g at 4° C.). Purity of protein was analyzed by Coomassie staining.

Biotinylated RNA Pulldown Assay

Genomic DNA was extracted from cell cultures to generate amplicons corresponding the 5′ and 3′ ends of the INCA1 intron 1. PCR products were cloned in pCR2.1-TOPO (Thermo Fisher Scientific) to generate pCR2.1-INCA1 Intron 1 (5′ half) and pCR2.1-INCA1 Intron 1 (3′ half). PCR products from pCR2.1-INCA1 Intron 1 (3′ half) were generated to add HindIII linkers to the 3′ end and this fragment was cloned between SpeI and HindIII in pCR2.1-INCA1 Intron1 (5′ half) to generate a pCR2.1-INCA1 minigene. In vitro transcripts of biotinylated RNA were generated by PCR and numbered fragment 1-7 in a 5′ to 3′ direction. Each fragment allowed for transcription of a 300 nucleotide RNA, each with a 50 nucleotide overlap to the adjacent fragment. T7 promoter sequence was added by PCR. In vitro transcription reactions were performed using T7 HiScribe (New England Biolabs) according to manufacturer's instructions, except the final concentration of UTP was reduced to 7.5 μM and instead supplemented with 2.5 μM Biotin-16-UTP (Sigma Aldrich). Transcribed RNAs were extracted by acidic phenol chloroform extraction (Thermo Fisher Scientific) and precipitated with ammonium acetate. Unincorporated nucleotides from resuspended RNAs were removed by gel filtration chromatography through Illustra Microspin G-25 columns (GE Healthcare). Concentrations of each RNA was brought to 4 μM with DEPC-treated H₂O (Thermo Fisher Scientific). 1 μl of 4 μM biotinylated in vitro transcribed RNA was added to cell lysates and protein complexes allowed to assemble for 2 h at 4° C. After incubation, 10 μl of streptavidin-agarose (Thermo Fisher Scientific) were added and tumbled for an additional hour. Beads were washed 4 times with lysis buffer and complexes were eluted with 2×SDS loading buffer. Eluted proteins were resolved on 4-20% gradient gel (Bio-Rad) and assayed by western blotting. For RNA pulldown assays with blocking oligos, prior to preforming pulldown assay, 4 pmol of biotinylated RNA was incubated with indicated amount of blocking oligo in 20 μl of binding buffer (10 mM Tris [pH 7.9], 50 mM NaCl, 10 mM MgCl₂ 1 mM DTT). RNA/oligo mixture was incubated at 90° C. for 5 minutes and allowed to cool to room temperature for 30 minutes to facilitate annealing. The following primers were used in the assay: INCA1 Exon1 Forward: GTGCCTGCAGCCGGCGGG (SEQ ID NO:29); INCA1 Intron 1 Reverse: cttgcggccgctaaccgTCTTTCTTCTATTTCAGACTCCTCC (SEQ ID NO:30); INCA1 Intron 1 Forward: cggttagcggccgcaagCAATGAAGCATGCAGGTCTGGG (SEQ ID NO:31); INCA1 Exon 2 Reverse: CTTGTCATTTCATGTCTCTTTAATTAAAGAG (SEQ ID NO:32); pCR2.1—HindIII Reverse: ggttaagcttAGATGCATGCTCGAGCGG (SEQ ID NO:33); Frag 1 Forward T7: taatacgactcactatagGGTGGGTCTGGCACGAAGG (SEQ ID NO:34); Frag 1 Reverse: TGTGACGGACGCAACTGG (SEQ ID NO:35); Frag 2 Forward T7: taatacgactcactataggAATACATGGTCCGTGCGG (SEQ ID NO:36); Frag 2 Reverse: CGCGGCCTGAGGAAGGGG (SEQ ID NO:37); Frag 3 Forward T7: taatacgactcactatagGCGGGAGGAGCGCGGAGGC (SEQ ID NO: 38); Frag 3 Reverse: CCCTAGACTCGCCCCACTTG (SEQ ID NO:39); Frag 4 Forward T7: taatacgactcactatagggGACCTAGGCAACGGCCT (SEQ ID NO:41); Frag 4 Reverse: ACCGGCAGGAGCTTCATCAC (SEQ ID NO:42); Frag 5 Forward T7: taatacgactcactataggCTTTAATGCCGGGGTCTG (SEQ ID NO:43); Frag 5 Reverse: AAGCTAAATATGGACCTACGCT (SEQ ID NO:44); Frag 6 Forward T7: taatacgactcactataggAGTTTTAGAACTGGAAGGG (SEQ ID NO:45); Frag 6 Reverse: TCTTTCTTCTATTTCAGACTCCTCC (SEQ ID NO:46); Frag 7 Forward T7: taatacgactcactataggCAATGAAGCATGCAGGTCTGGG (SEQ ID NO:47); Frag 7 Reverse: CTGCAAAAGAAAACAACAAAAAAACTAAAG (SEQ ID NO:48).

RNA Electrophoretic Mobility Shift Assay

Synthetic RNA was obtained from IDT and radiolabeled with γ-³²P ATP (6000 Ci/mmol, Perkin-Elmer) using T4 Polynucleotide Kinase (New England Biolabs). Unincorporated nucleotides were removed by gel filtration chromatography through Illustra Microspin G-25 columns (GE Healthcare). RNA/protein complexes were allowed to form at room temperature by adding indicated amount of protein to 1 pmol of radiolabeled RNA in 20 μl reaction containing 50 mM Tris [pH 8.0], 300 mM KCl, 0.02% NP-40, 10% Glycerol, 0.5 μg/μl Heparin for 10 minutes. Complexes were loaded onto native polyacrylamide gels and ran for 3 hours at 150 V. Gels were dried and visualized by autoradiography.

Microscale Thermophoresis

MicroScale Thermophoresis experiments were performed according to the NanoTemper technologies protocol in a Monolith NT.115Pico instrument (NanoTemper Technologies). Serial dilutions of HNRNPH1 were done using a buffer containing 50 mM Tris [pH 8.0], 300 mM KCl, 0.02% NP-40, 10% Glycerol, and 0.5 mg/ml Heparin. RNA oligos were labelled with a FAM moiety at their 3′ ends (IDT). The RNA concentration was kept constant at 20 nM throughout the experiments. The RNA-protein mixture was incubated at room temperature for 15 mins before running into the MST instrument. The experiments were performed using 40% and 60% MST power and between 20-80% LED power at 22° C. The MST traces were recorded using the standard parameters: 5 s MST power off, 30 s medium MST power on and 5 s MST power off. The reported measurement values are the combination of two effects: the fast, local environment dependent responses of the fluorophore to the temperature jump and the slower diffusive thermophoresis fluorescence changes. The data presented here are the average of 3 independent experiments. Average normalized fluorescence (%) was plotted against HNRNPH1 concentration to determine the binding constant (K_{d}). Ligand depletion model with one binding site was used (Using GraphPad Prism 8) to fit the binding which follows the following model: Y=Bmax*X/(Kd+X).

### Example 1. Tumor Interferon Signaling is Regulated by a Cis-Acting lncRNA INCA1 Transcribed from the PD-L1 Locus

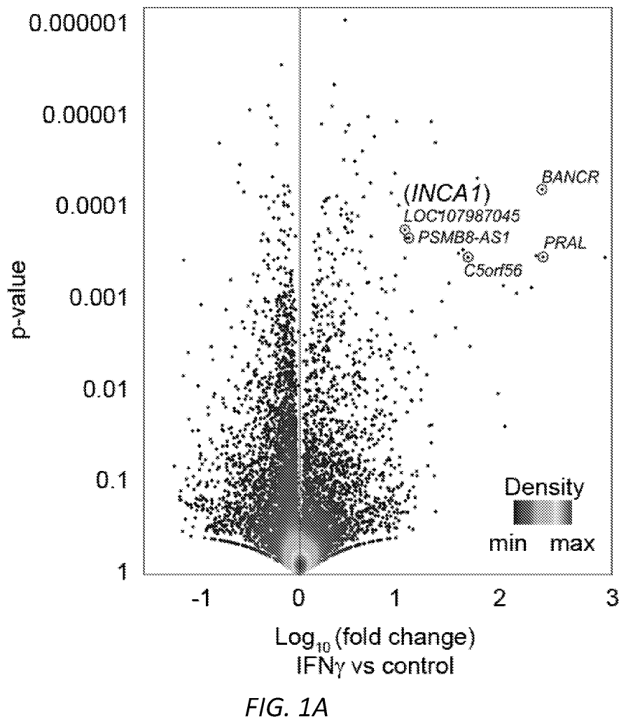

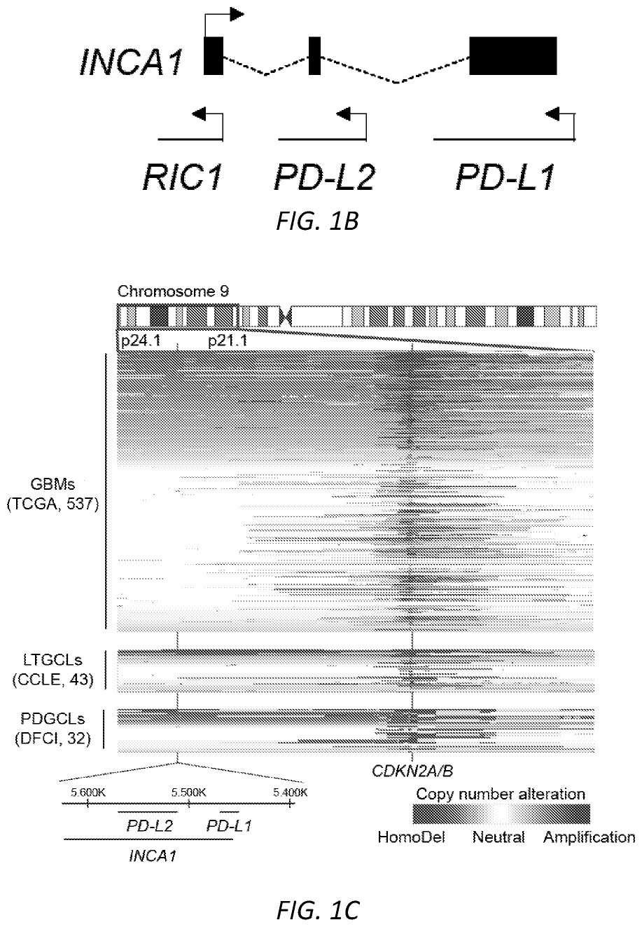

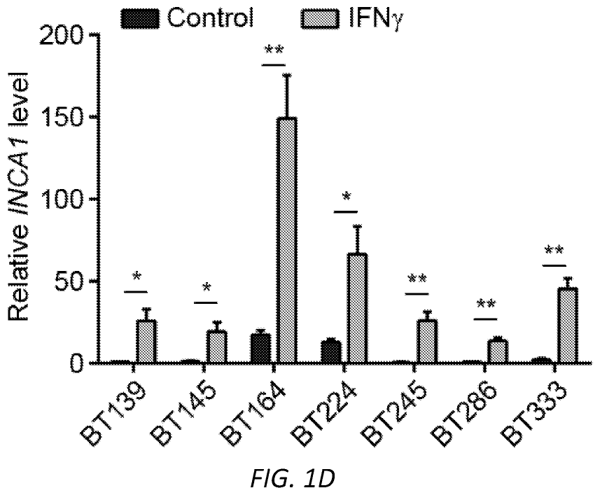

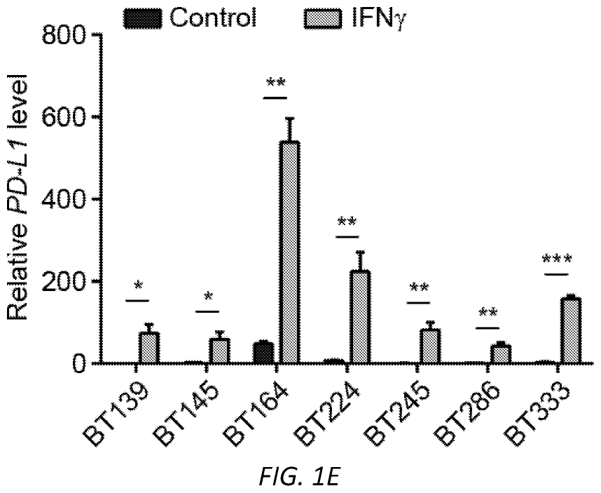

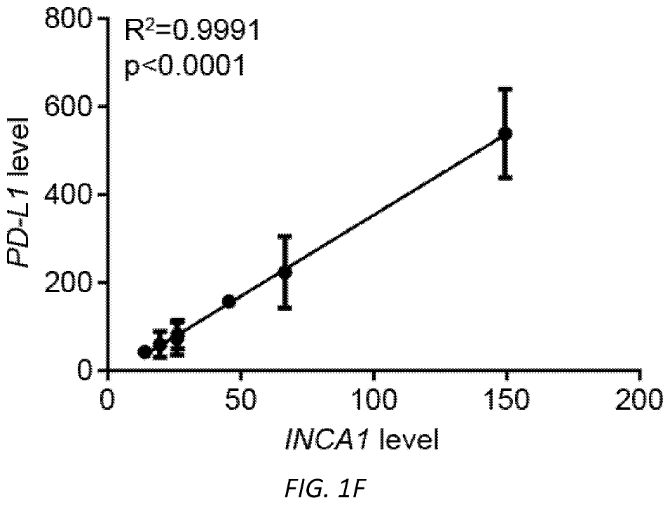

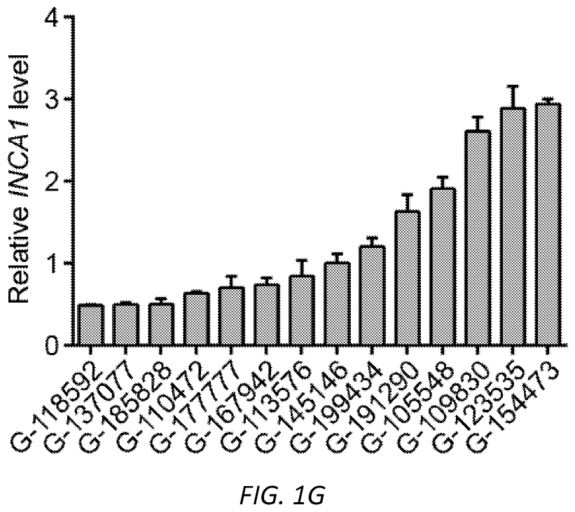

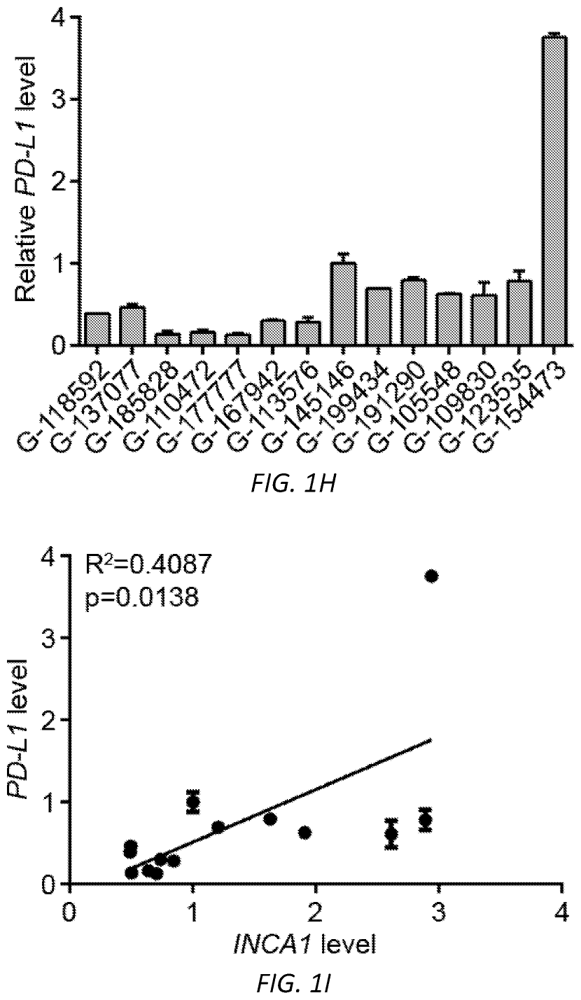

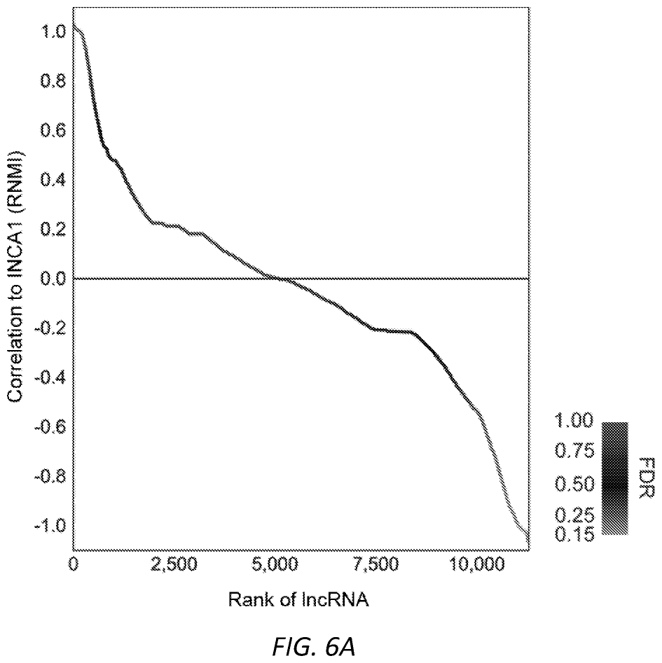

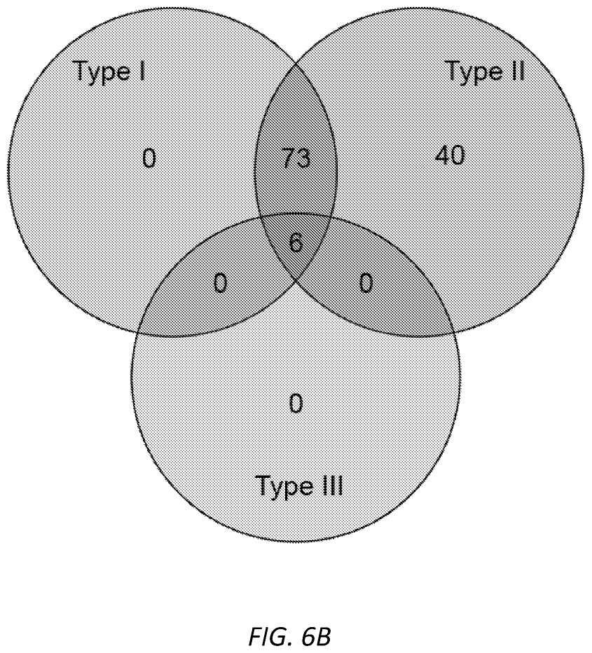

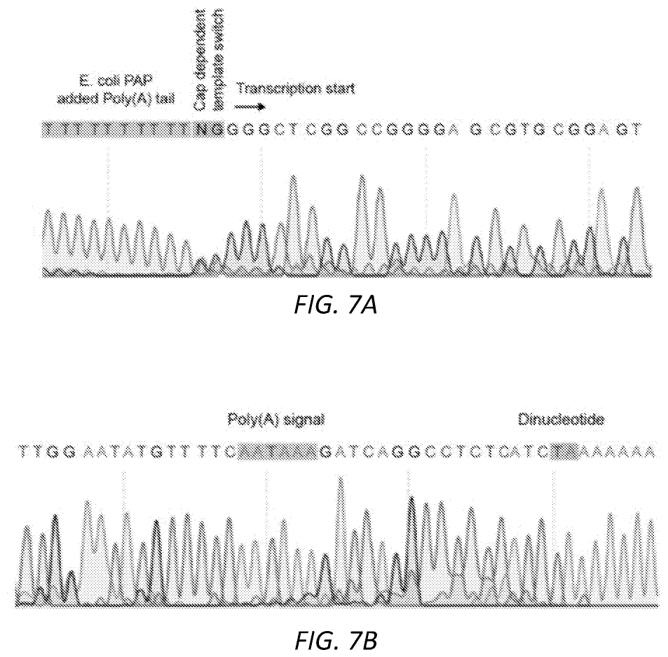

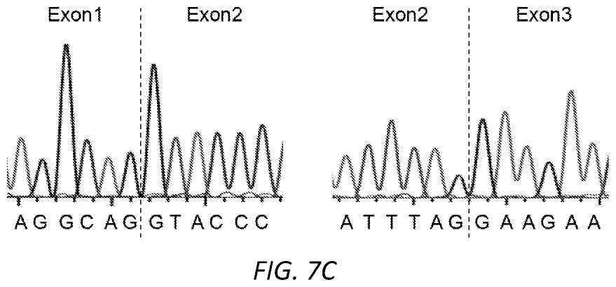

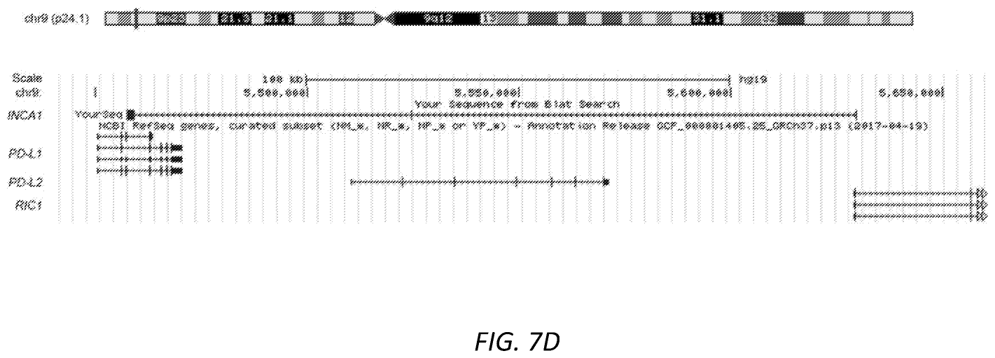

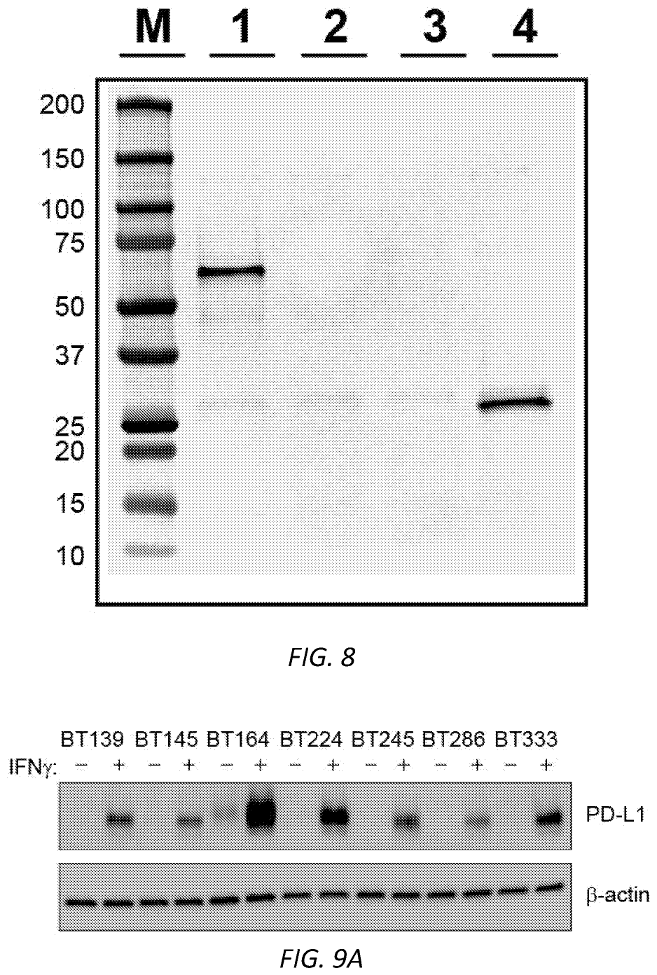

To identify tumor lncRNAs with immunomodulatory functions, whole-transcriptome analysis (RNA-seq) of patient-derived glioblastoma (GBM) cell lines (PDGCLs) stimulated with IFNγ was performed (FIG. 1A). IFNγ stimulated the transcription of 113 lncRNAs (p<0.01, fold change >2) including BANCR, a lncRNA previously shown to be upregulated by IFNγ (15), validating the approach. Among the most up-regulated lncRNAs was a novel lncRNA expressed from the opposite DNA strand of the PD-L1/PD-L2 locus (FIG. 1). Due to its IFNγ-dependent expression, we named this lncRNA Interferon-γ-stimulated Non-Coding RNA 1 (INCA1). INCA1 expression was positively correlated to the expression of 237 other lncRNAs (FDR≤0.25), several of which were transcribed from loci of protein-coding genes known to be IFNγ regulated (FIGS. 6A-B). Previously annotated in RefSeq as LOC107987045 (the predicted RNA was previously, but is not presently, annotated in NCBI as XR_001746611.1, and is an annotated in Ensembl as ENSG00000286162.2), the INCA1 gene was predicted to span a genomic region of 172.5 kilobases (kb) located in chr9p24.1 that produces a spliced lncRNA of about 2 kb. Using 5′ and 3′ rapid amplification of cDNA ends (RACE), we identified the 5′ and 3′ ends of the INCA1 transcript. RACE sequencing data demonstrated that INCA1 has a canonical polyadenylation signal at the 3′ end whose coordinates are chr9:5,629,748-5,457,434 (FIGS. 7A and 7B). Moreover, we performed PCR amplification to obtain the full sequence of the INCA1 transcript. Sequencing PCR products from 3 different cell lines revealed that INCA1 is a 2,030 nt long lncRNA, composed of 3 exons (FIG. 7C and SEQ ID NO:1). The 5′ end of the INCA1 transcript consists of a 182 nt exon located within the first intron of the RIC1 gene. This is followed by a short 94 nt long exon located in the antisense orientation of the second intron of the PD-L2 gene and finally a 3′ end exon of 1,754 nt found in an antisense direction of the third intron of the PD-L1 gene (FIG. 7D). We also confirmed that this INCA1 isoform was not translated into protein, using an in vitro transcription/translation assay (FIG. 8). Analysis of the INCA1 locus, and the nearby 9p21 cytoband that includes the CDKN2A/B genes, in 32 PDGCLs and 43 long-term GBM cell lines (LTGCLs) revealed a pattern of copy number alterations similar to GBM tumors from the TCGA cohort, suggesting that our experimental cell line models reflected the human disease (FIG. 1C).

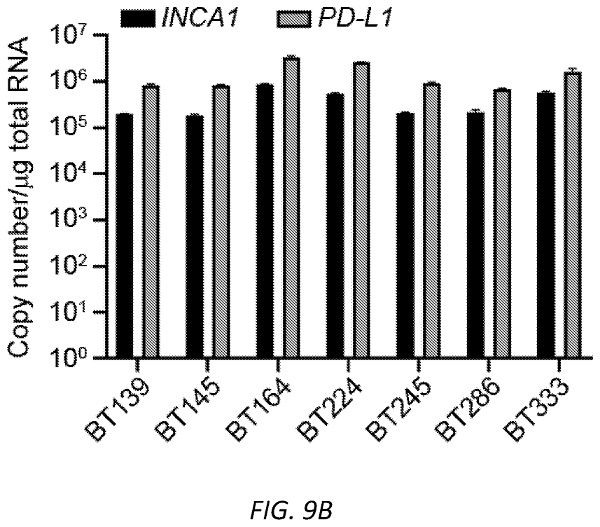

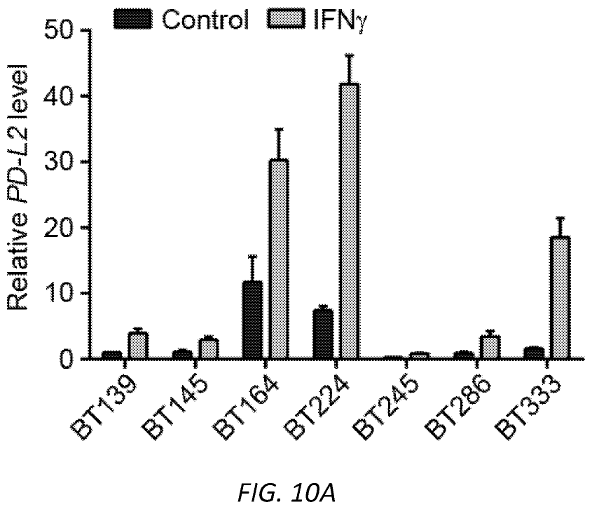

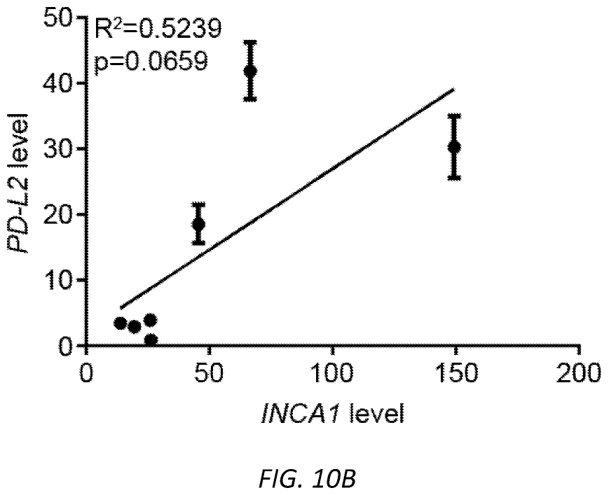

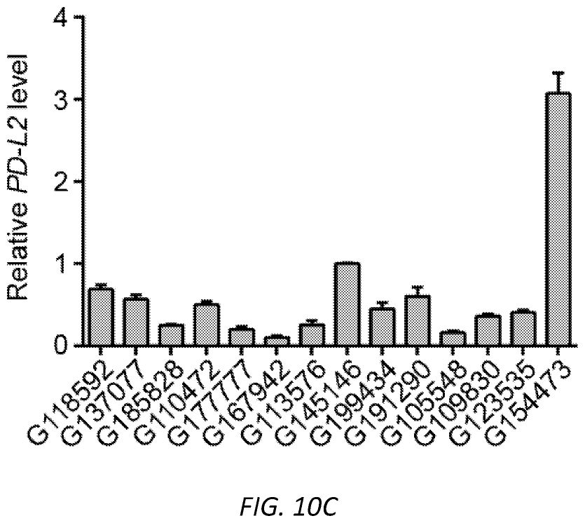

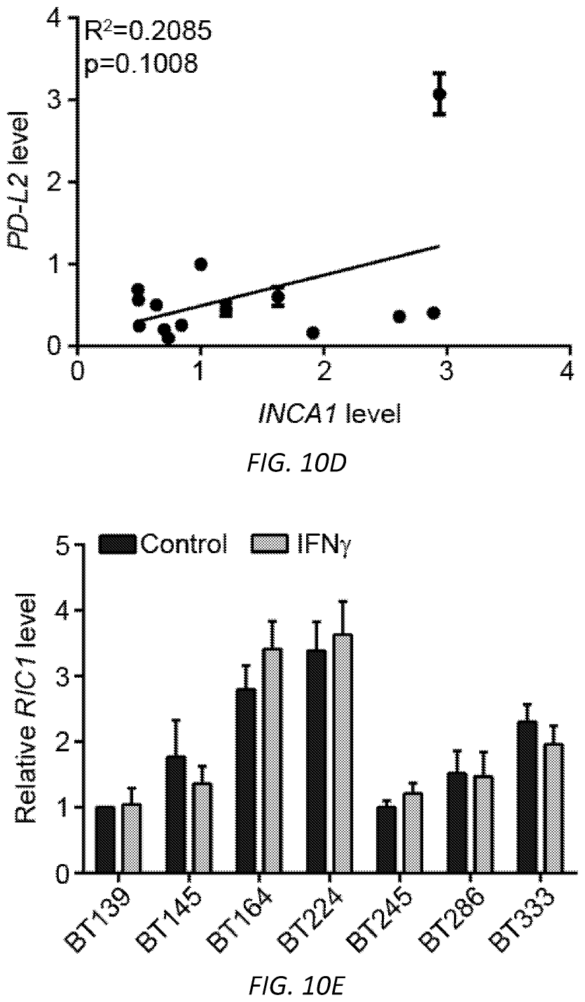

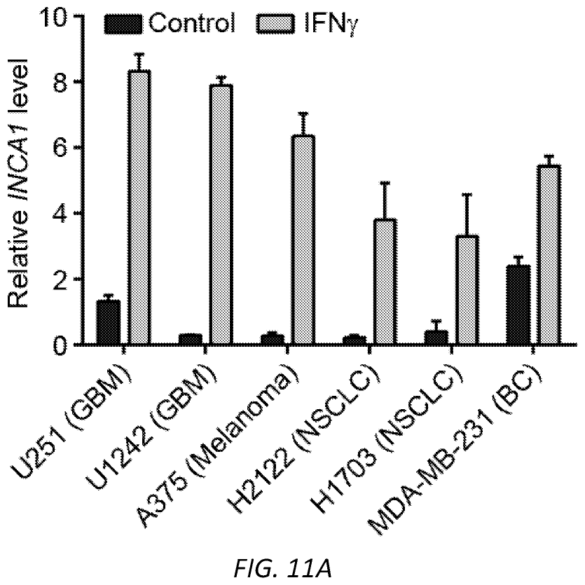

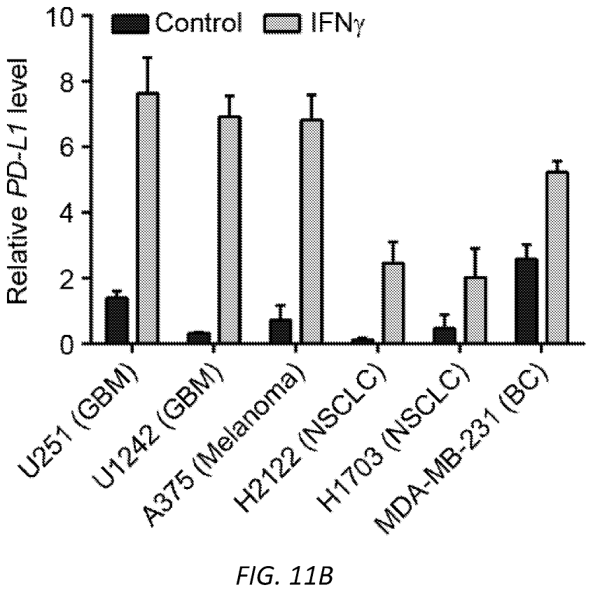

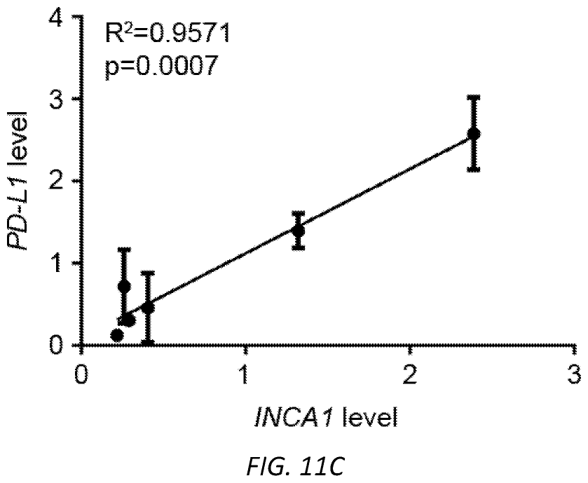

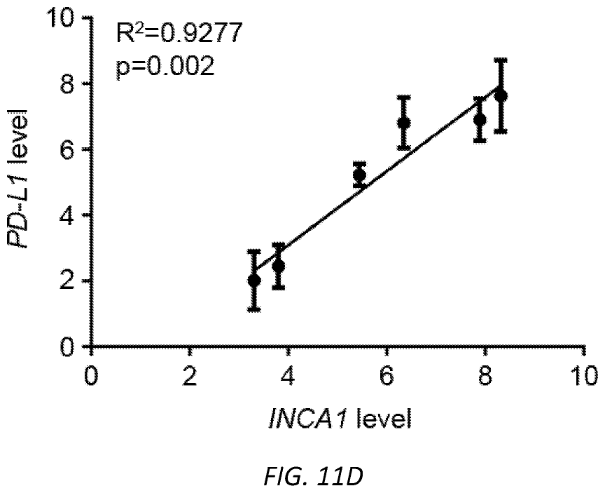

To validate the RNA-seq data, we performed qPCR analysis in a select number of PDGCLs (n=7). In all lines, INCA1 was up-regulated in response to IFNγ stimulation (FIG. 1D). IFNγ-treated PDGCLs also expressed high levels of PD-L1 mRNA and protein (FIG. 1E and FIG. 9A), with only minor differences in RNA copies compared to INCA1 (FIG. 9B). Notably, we observed a highly significant correlation between INCA1 and PD-L1 expression (FIG. 1F). Consistent with these findings, INCA1 expression correlated with PD-L1 mRNA levels in patient GBMs, with INCA1-high tumors also expressing higher PD-L1 levels (FIGS. 1G-I). We also observed up-regulation of PD-L2 in PDGCLs stimulated with IFNγ (FIG. 10A). However, there was no significant correlation between PD-L2 and INCA1 expression, in either PDGCLs or GBM patient tumors (FIGS. 10B-D). In addition, no correlation in expression was observed between INCA1 and its third overlapping gene RIC1 (FIG. 1B and FIG. 10E). To determine if the correlation between INCA1 and PD-L1 expression extended beyond our PDGCL model, we analyzed the response to IFNγ treatment in cells from other cancers. Our panel included 6 cell lines representing GBM, melanoma, non-small cell lung cancer (NSCLC) and breast cancer (BC). All showed increased expression of INCA1 after IFNγ stimulation (FIG. 11A) that also correlated with PD-L1 expression, both in untreated and IFNγ-treated cells (FIGS. 11B-D).

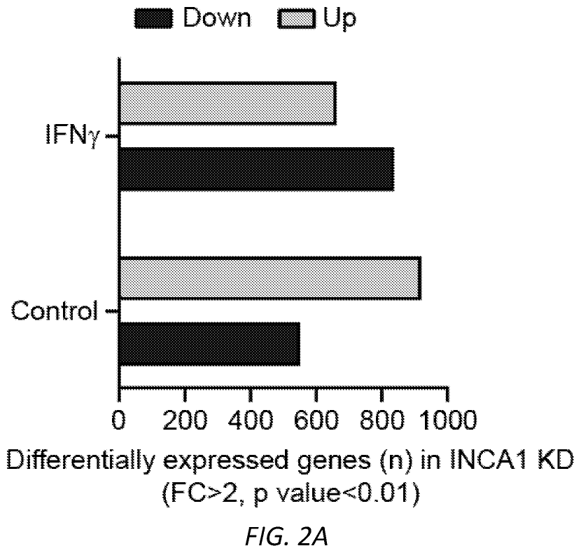

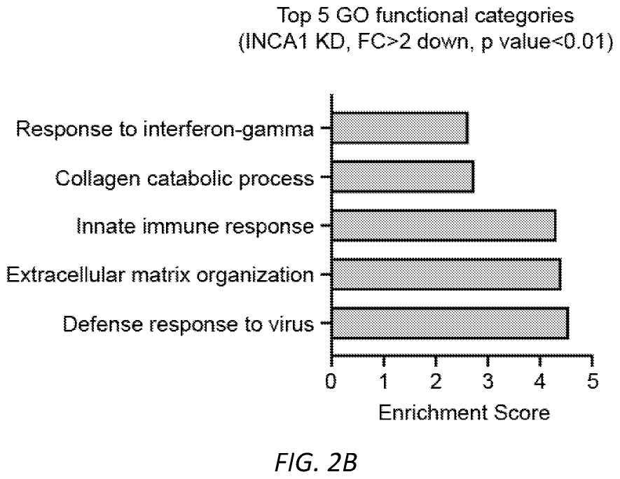

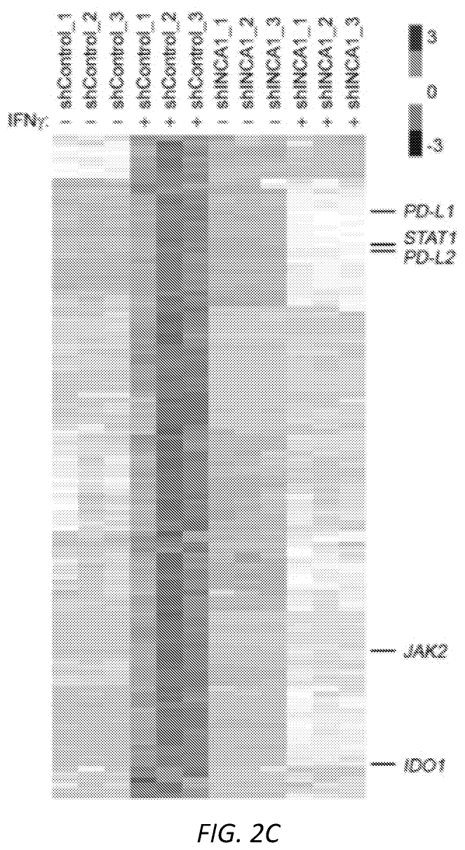

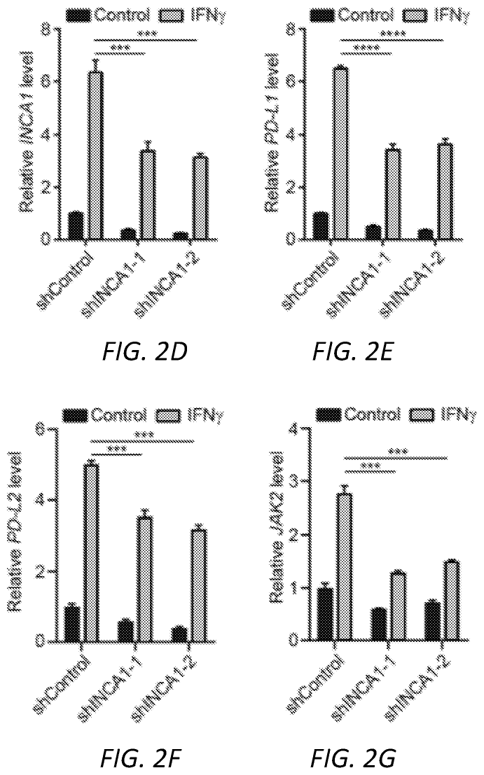

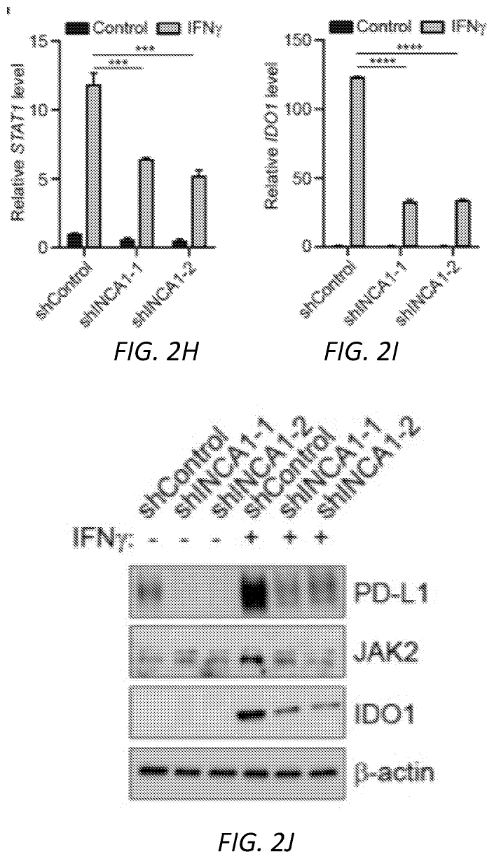

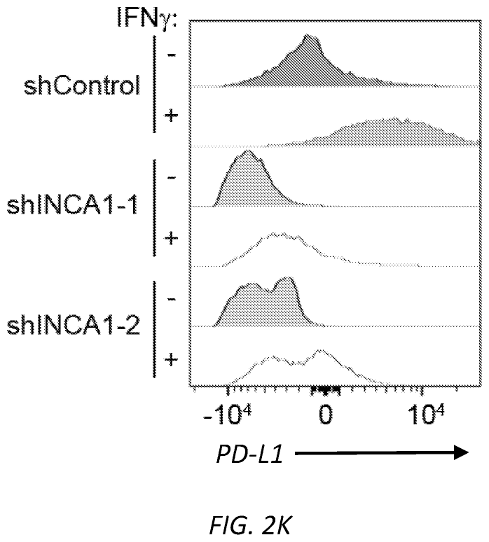

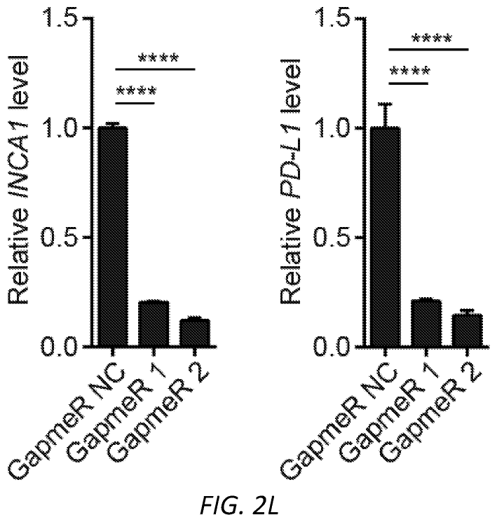

Since several lncRNAs act as major modulators of gene expression in response to stimuli (Atianand et al., 2016; Mineo et al., 2016), we hypothesized that INCA1 would regulate the expression of its neighboring genes and the tumor response to IFNγ stimulation. To test our hypothesis, we generated stable knockdown of INCA1 in the U251 GBM cell line using short hairpin RNAs (shRNAs), and we assessed the impact of INCA1 silencing on global gene expression by RNA-seq. Silencing INCA1 altered the expression of 1,501 genes (p<0.01, fold change >2, FIG. 2A). Gene ontology (GO) enrichment analysis showed downregulation of genes involved in immune-related functions, such as IFNγ response, innate immune response and defense response to virus (FIG. 2B). Moreover, INCA1 knockdown resulted in reduced expression of 124 ISGs (FIG. 2C). Among those genes were important components of the IFNγ signaling pathway (JAK2 and STAT1), as well as major immunosuppressive molecules (PD-L1 and IDO1). We further validated the deregulated expression of selected genes by qPCR. Our data confirmed that silencing INCA1 resulted in reduced mRNA levels of its overlapping genes PD-L1 and PD-L2, both at basal level and in response to IFNγ (FIGS. 2D-F). Notably, the mRNA of JAK2, whose gene is in the same locus as INCA1 and PD-L1, was significantly downregulated (FIG. 2G), along with ISGs from different genomic regions, such as STAT1 and IDO1 (FIGS. 2H and 2I). Moreover, we showed that silencing INCA1 resulted in downregulation of PD-L1, JAK2 and IDO1 protein levels (FIG. 2J). Silencing INCA1 reduced both total PD-L1 protein and the levels of cell surface PD-L1 (FIG. 2K). Moreover, silencing INCA1 expression in PDGCLs using chemically modified antisense oligonucleotide (gapmers) resulted in a 5- to 7-fold reduction in PD-L1 expression compared to cells transfected with a scrambled control gapmer (FIG. 2L).

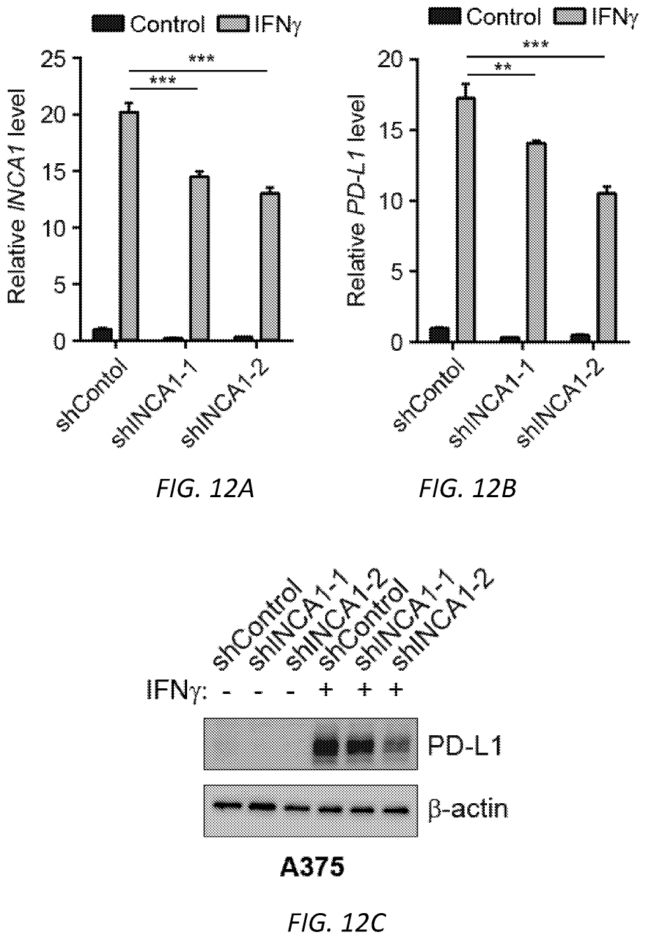

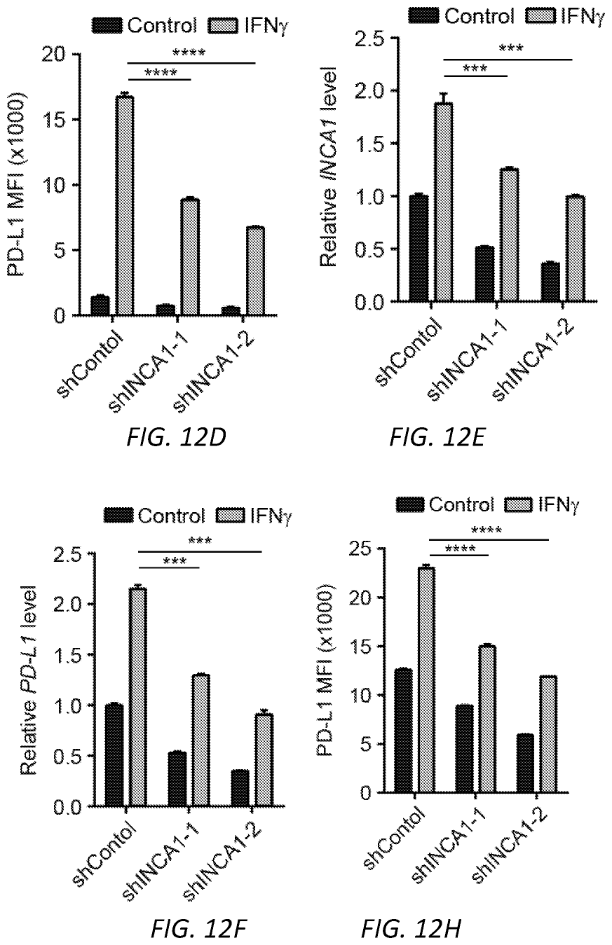

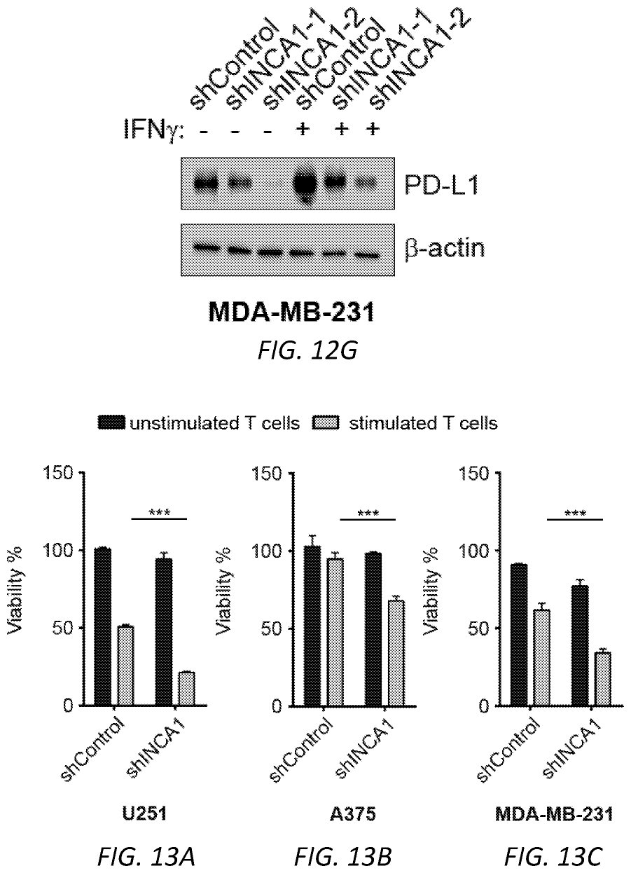

Since PD-L1 expression significantly contributes to cancer-associated immunosuppression (Beatty and Gladney, 2015), to assess whether INCA1 is able to regulate PD-L1 expression in tumors other than GBM, we selected two non-GBM cell lines with different basal levels of PD-L1 to generate stable INCA1 knockdowns; the A375 melanoma cell line, which showed no detectable basal levels of PD-L1 by immunoblot, and the MDA-MB-231 breast cancer cell line, which exhibited the highest basal levels of PD-L1 among the cell lines analyzed. INCA1 knockdown in A375 cells led to a significant reduction of IFNγ-mediated PD-L1 expression compared to control cells (FIGS. 12A-C). Silencing INCA1 in MDA-MB-231 cells resulted in a significant decrease of PD-L1 basal levels and a strongly attenuated response to IFNγ stimulation (FIGS. 12E-G). Moreover, both cell lines showed reduced levels of PD-L1 at their surface when INCA1 was silenced (FIGS. 12D and 12H). Together, these results indicate that INCA1 modulates tumor response to IFNγ treatment by regulation of multiple ISGs in GBM and in other cancer types.

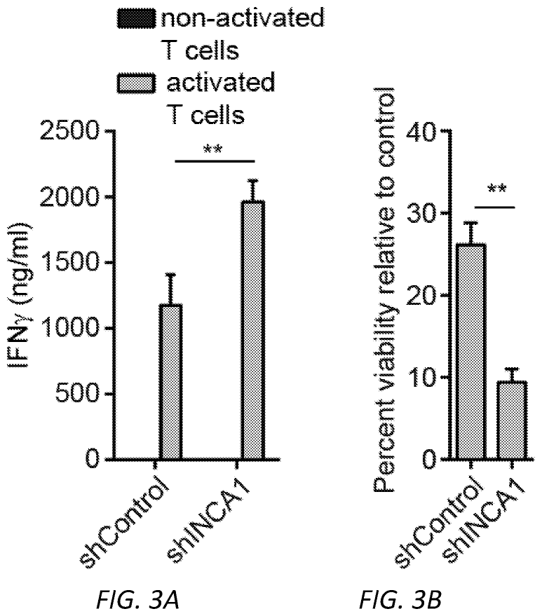

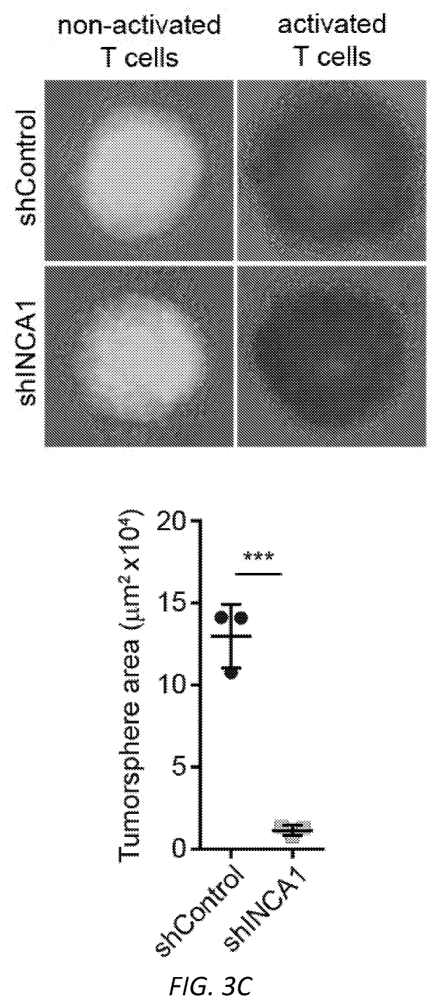

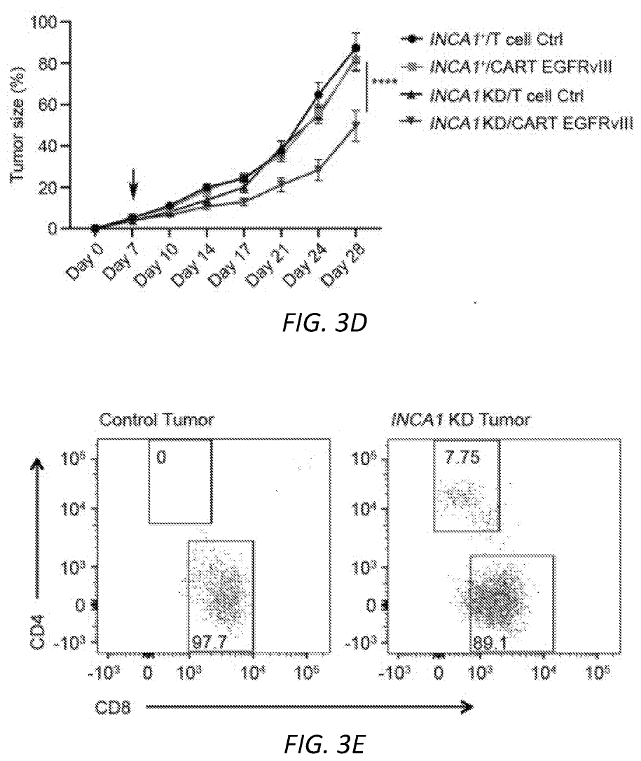

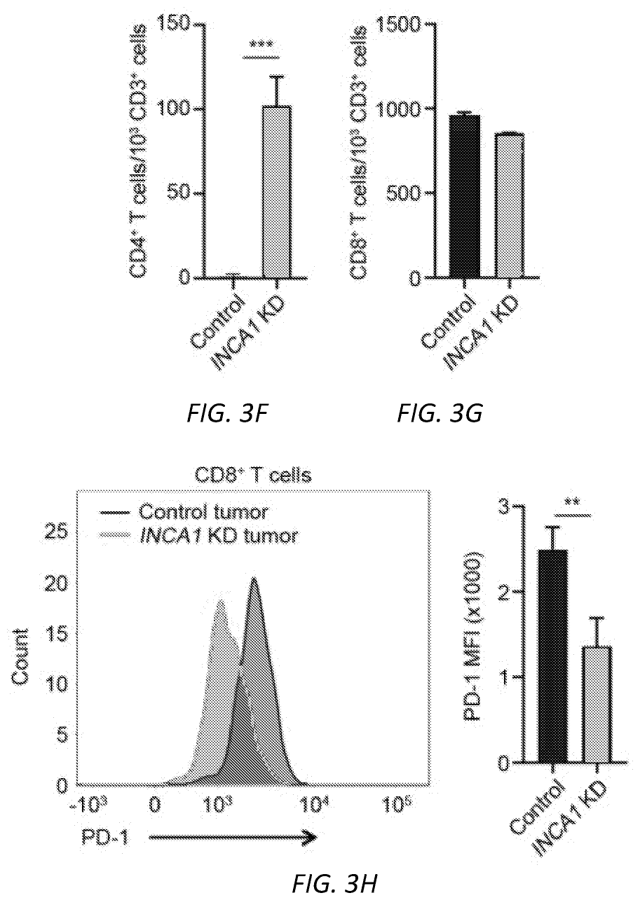

CD8⁺ cytotoxic T lymphocytes (CTLs) act as important effectors of cancer immunoediting (Mittal et al., 2014). Activation of CD8⁺ CTLs induces the secretion of cytokines, such as IFNγ, which promote their proliferation and anti-antitumor activity (Bhat et al., 2017; Zhang and Bevan, 2011). Because INCA1 silencing reduced the expression of major IFNγ-regulated immune inhibitory molecules in tumor cells, we tested whether silencing INCA1 would increase CD8⁺ CTL activity. Using a 2D culture system, we found that CD8⁺ CTLs co-cultured with INCA1 knockdown cells generated significantly more IFNγ when compared to CD8⁺ CTLs co-cultured with control cells (FIG. 3A). This was associated with increased cytotoxicity of INCA1 knockdown cells mediated by activated CD8⁺ CTLs (FIG. 3B). We further confirmed the greater activity of CD8⁺ CTLs in killing U251, A375 and MDA-MB-231 cells silenced for INCA1 using live/dead staining and FACS analysis (FIGS. 13A-C). Further, using a 3D culture system, we found a significant reduction in the size of INCA1 knockdown tumor spheres compared to control tumor spheres when these were co-incubated with activated CD8⁺ CTLs (FIG. 3C).

T cells engineered to express a Chimeric Antigen Receptor (CAR) against a specific tumor antigen are a potential curative therapy for different cancer types, but has produced only modest success in solid tumors mainly due to the highly immunosuppressive microenvironment (D'Aloia et al., 2018). To test if silencing INCA1 could improve CAR T cell function in vivo, U251-EGFRvIII control and INCA1 knockdown tumors were implanted subcutaneously. Seven days after implantation, human T cells expressing GFP alone (control) or the EGFRvIII-directed CAR were injected with a single intravenous dose that is about 1/10 of the dose that is standard (Hillerdal et al., 2014; Song et al., 2015; Wing et al., 2018; Zhang et al., 2019). Under these conditions, mice with control tumors showed no significant response to the CAR T cell therapy. In contrast, CAR T cells significantly reduced tumor growth in mice bearing INCA1 knockdown tumors (FIG. 3D). At the end of the study, twenty-one days after T cell injection, tumors were analyzed for the presence of CAR T cells. INCA1 knockdown tumors presented infiltrates of both CD4⁺ and CD8⁺ T cells with a predominance of CD8⁺ T cells. In contrast, no CD4⁺ T cell infiltrates were observed in control tumors (FIGS. 3E-G). Furthermore, CD8⁺ T cells in control tumors expressed a significantly higher level of PD-1 compared to those infiltrating tumors with silenced INCA1 (FIG. 3H). Taken together, these results show that INCA1 plays a functional role in controlling tumor IFNγ signaling, and that its knockdown leads to increased susceptibility of human tumor cells to T cell-mediated killing.

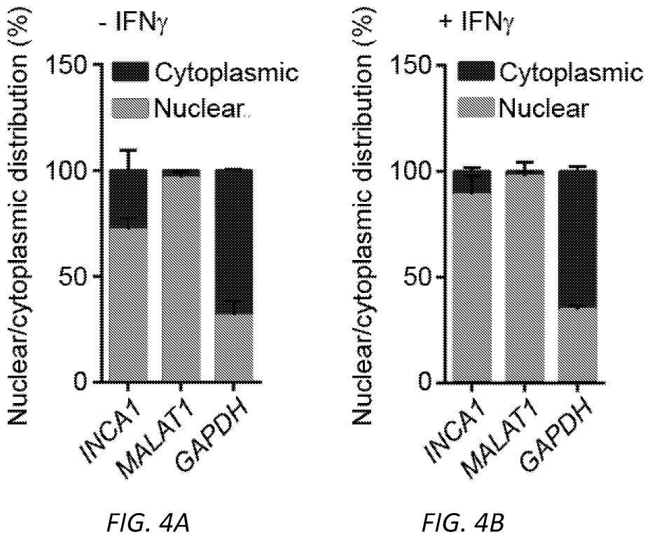

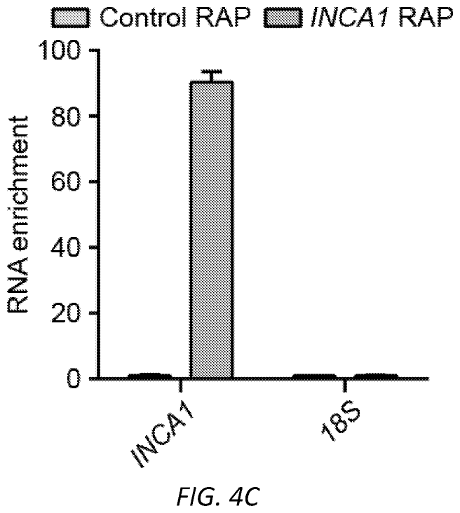

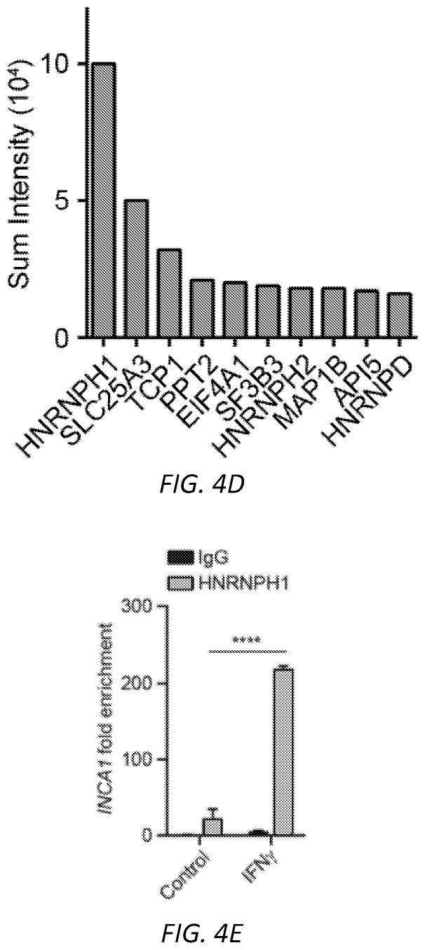

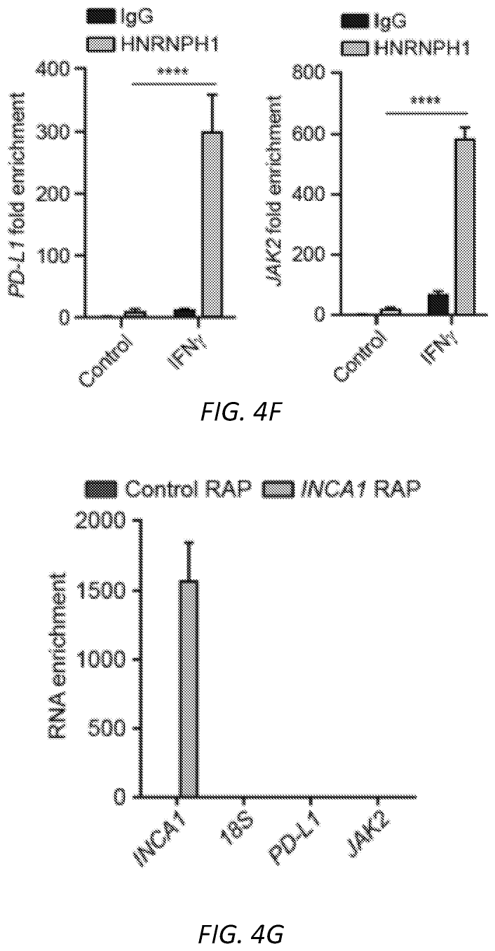

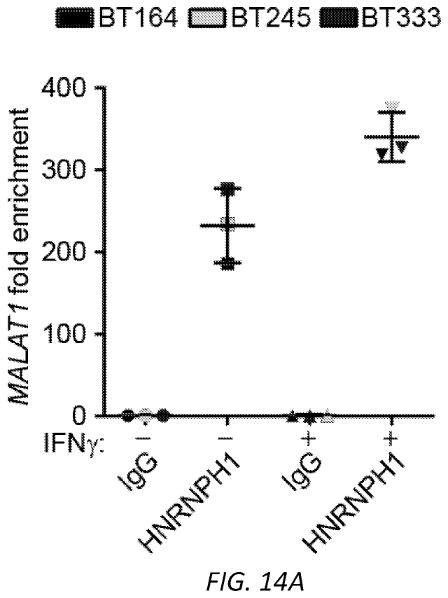

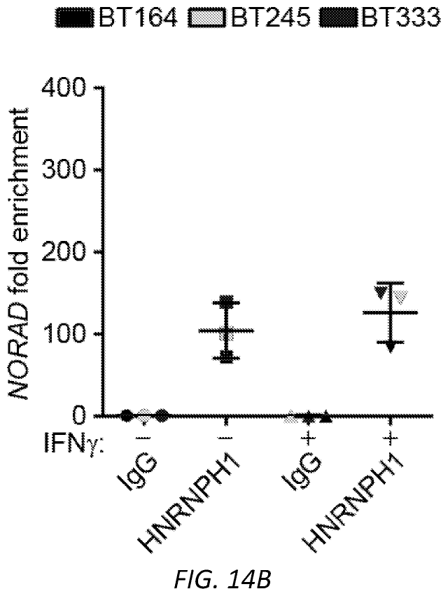

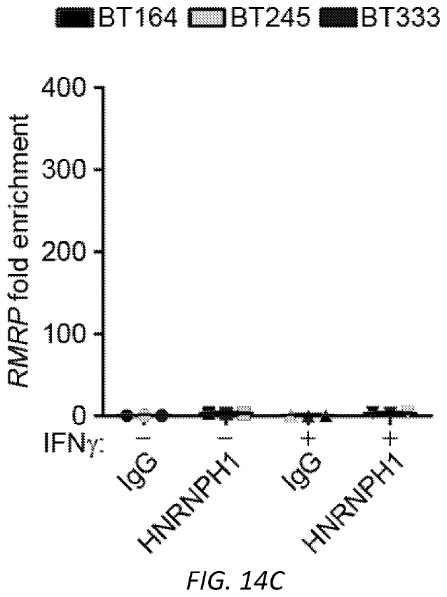

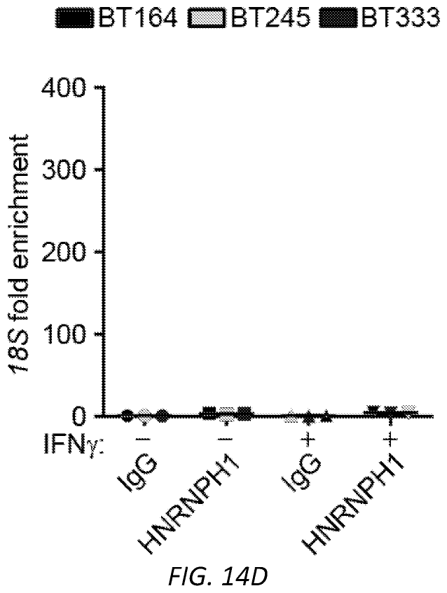

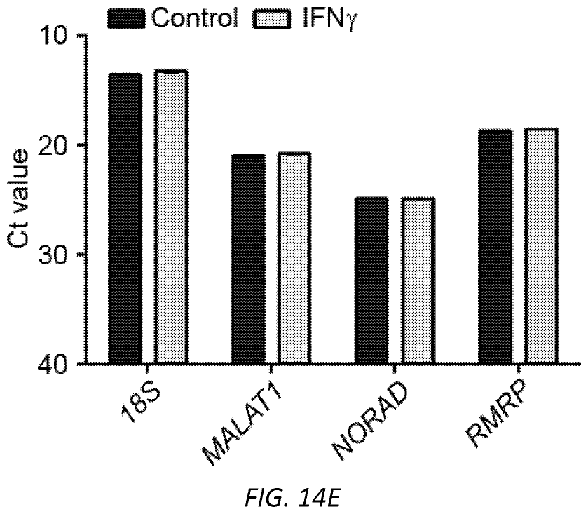

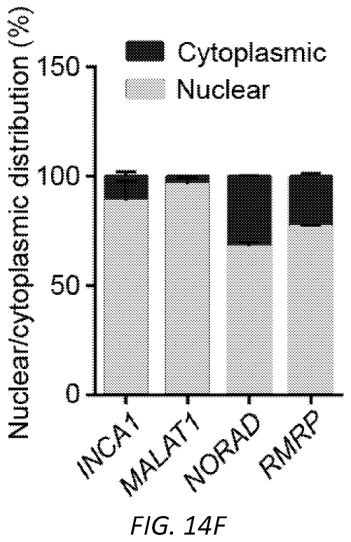

Most lncRNAs have been shown to function through their interaction with proteins, such as transcription factors or heterogeneous nuclear ribonucleoproteins (hnRNPs) (20-23). To identify the molecular mechanism through which INCA1 regulated PD-L1 expression, we first examined localization of INCA1 in PDGCLs using qPCR analysis of cellular fractions. We found that INCA1 was mostly nuclear in untreated cells, and nuclear localization increased after cells were stimulated with IFNγ (FIGS. 4A-B). To identify proteins that interact with INCA1, we performed RNA Antisense Purification (RAP) to purify endogenous INCA1 and proteins with which it is in direct contact in IFNγ-stimulated PDGCLs. We designed probes covering the entire INCA1 sequence, crosslinked RNA-protein complexes by UV irradiation and performed RAP in denaturing conditions to maximize the recovery of specific RNA-protein interactions. We observed greater than 80-fold enrichment in INCA1 RNA compared to control purification (FIG. 4C) and identified HNRNPH1 as the top hit by mass spectrometry (FIG. 4D). We then validated the interaction between INCA1 and HNRNPH1 by in vivo RNA-immunoprecipitation of UV-crosslinked samples (CLIP) using three different PDGCLs. To increase the strength of our validation, we included two lncRNAs previously demonstrated to be binding partners of HNRNPH1 (MALAT1 and NORAD) and one lncRNA known to not bind HNRNPH1 (RMRP) (FIGS. 14A-D) (24). We showed that all the lncRNAs tested were highly expressed in PDGCLs (FIG. 14E) and their localization was mostly nuclear (FIG. 14F). qPCR analysis of RNA co-purified with HNRNPH1 confirmed binding of HNRNPH1 to MALAT1 and NORAD and the absence of interaction with RMRP and 18S, and showed significant binding of INCA1 in IFNγ-stimulated cells compared to untreated cells and isotype control (FIG. 4E). Because INCA1 is expressed from a locus containing IFNγ-stimulated coding genes, we tested if mRNAs of those genes were also bound to HNRNPH1. Our data showed that HNRNPH1 bound PD-L1 and JAK2 mRNA (FIG. 4F), but did not bind PD-L2 mRNA. Moreover, to assess if INCA1 was bound to HNRNPH1 in complex with PD-L1 and/or JAK2 mRNA, we performed RAP-RNA analysis to identify RNA-RNA interactions. Using this approach, we did not detect direct or indirect binding between INCA1 and PD-L1 or JAK2 mRNA (FIG. 4G). These results indicate that INCA1 binding to HNRNPH1 was independent of PD-L1 and JAK2 mRNA binding.

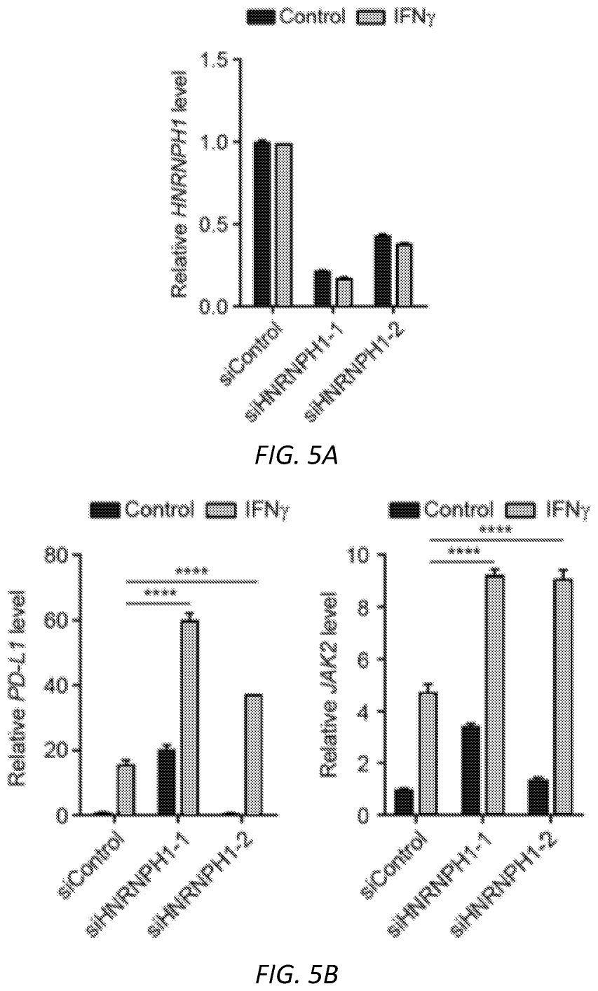

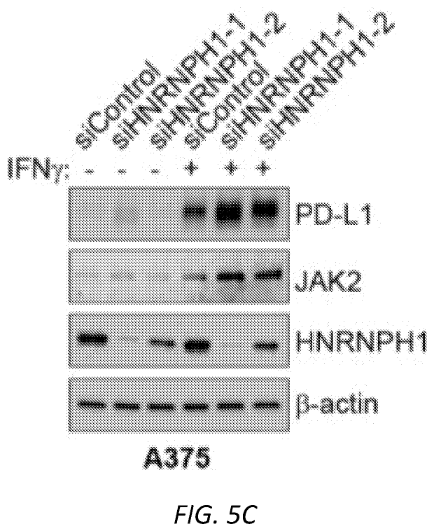

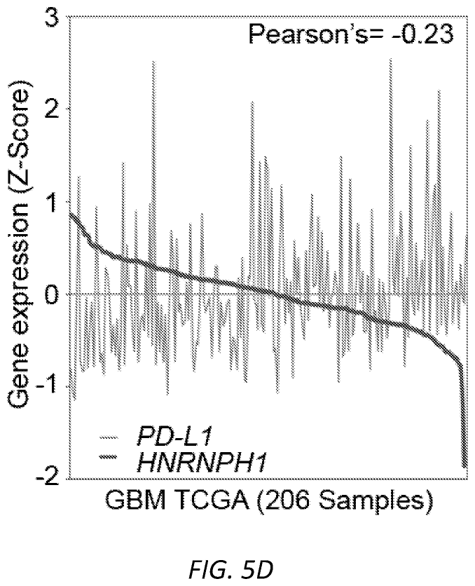

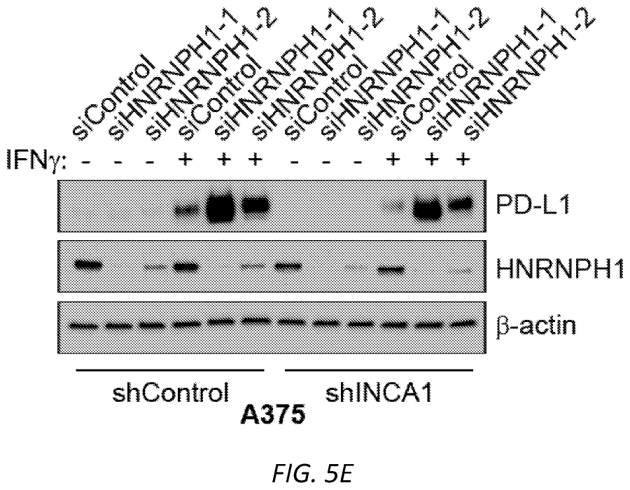

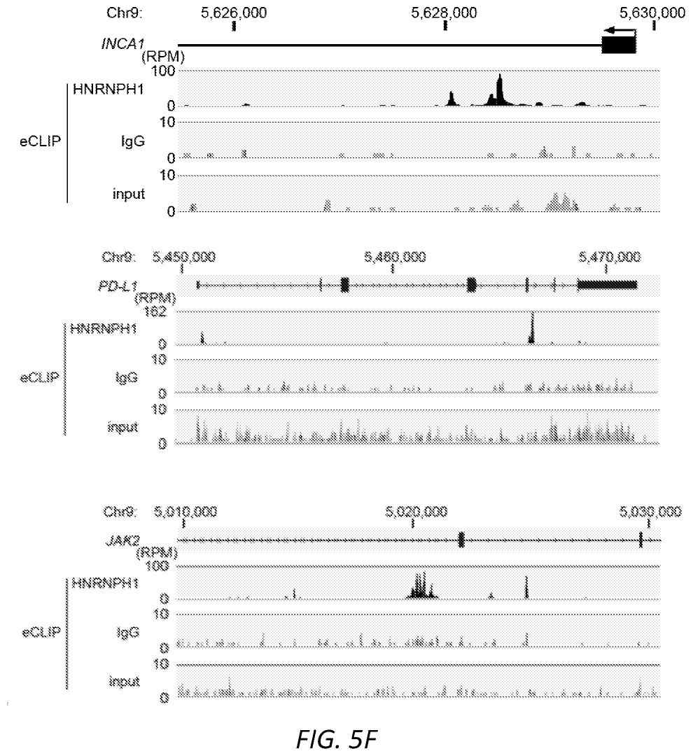

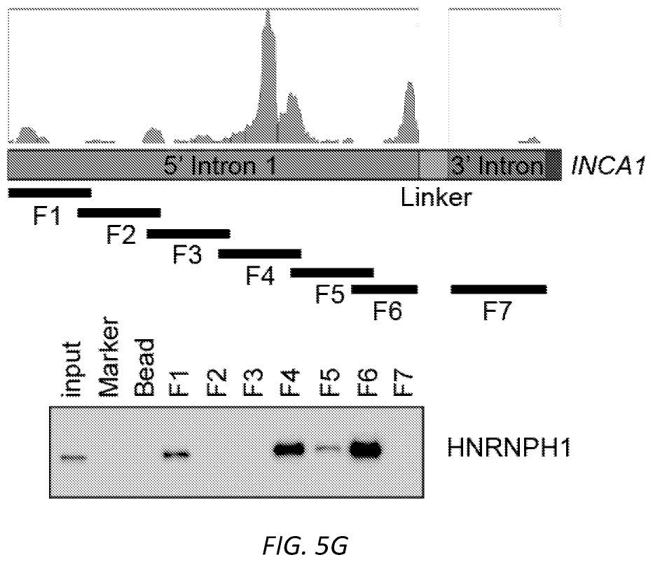

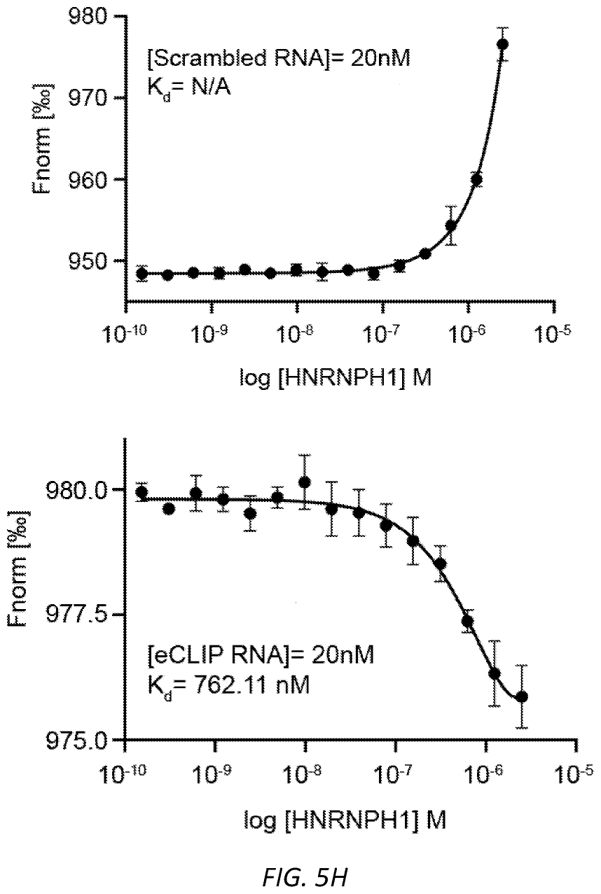

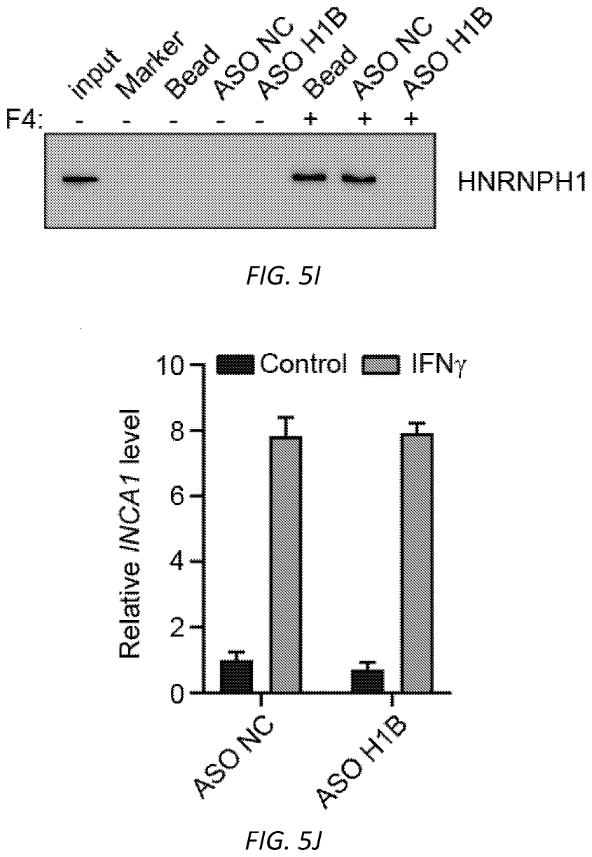

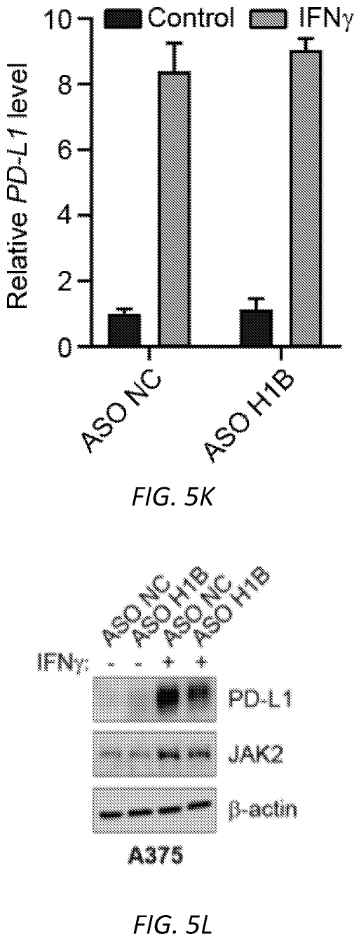

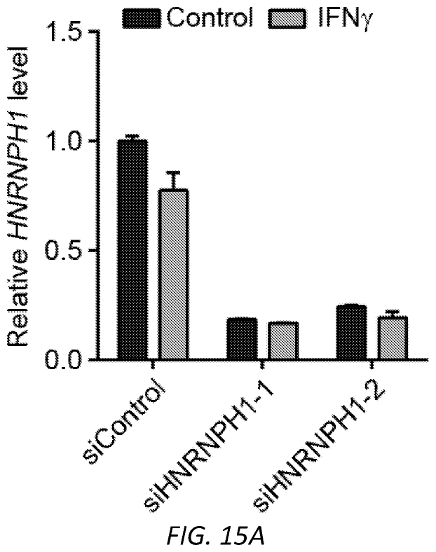

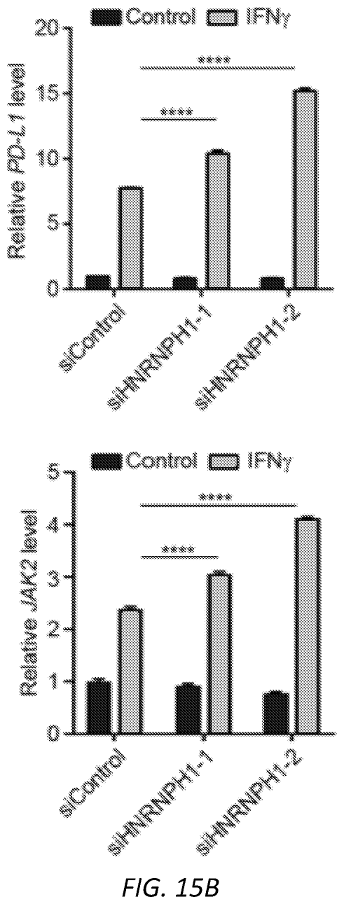

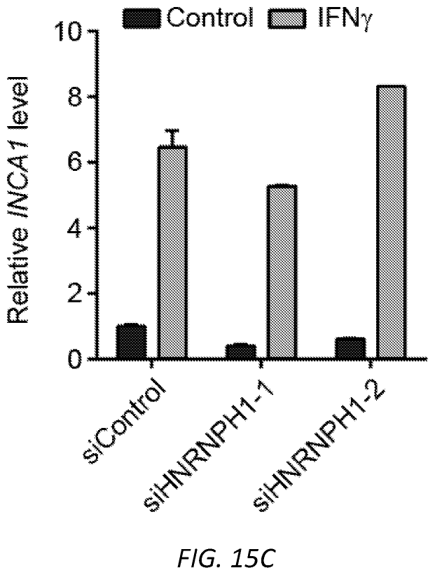

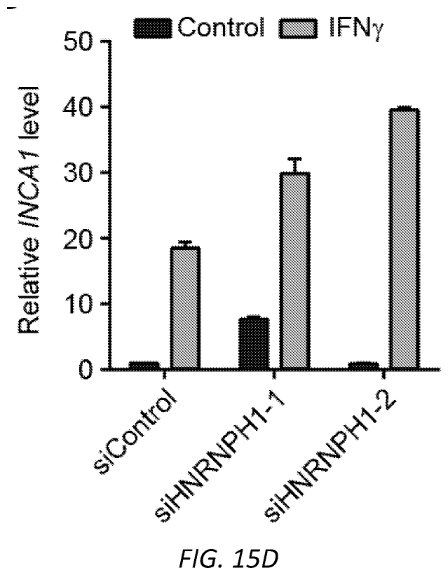

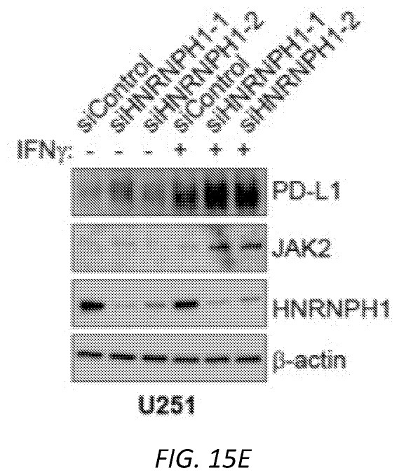

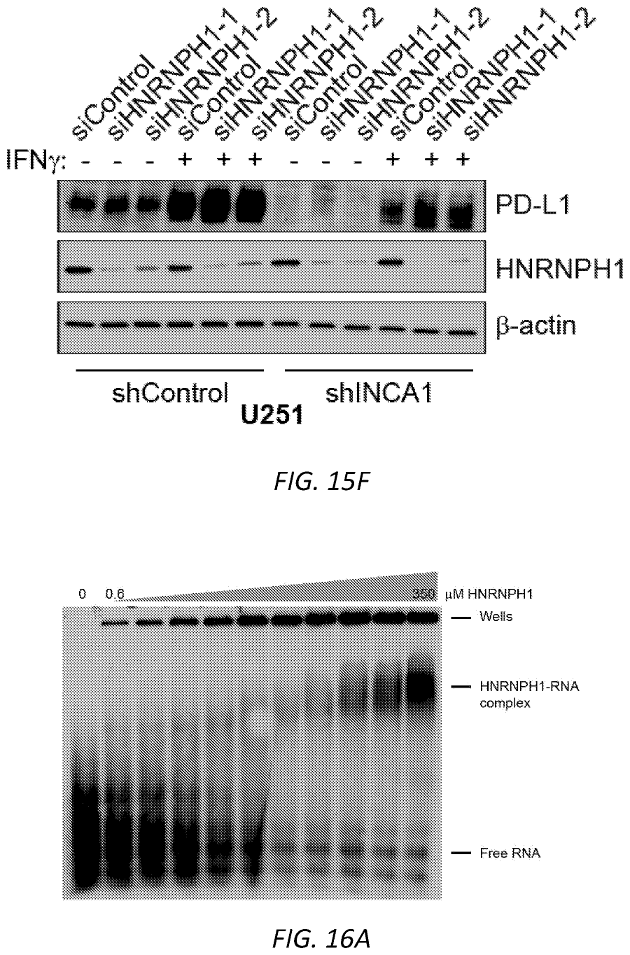

The observed absence of INCA1 binding to PD-L1 and JAK2 transcripts coupled with the observed binding of INCA1, PD-L1 and JAK2 transcripts to HNRNPH1 led us to hypothesize that INCA1 acted as a decoy RNA that competitively inhibits HNRNPH1 binding to PD-L1 and JAK2 transcripts. To test our hypothesis, we first investigated the effects of modulating HNRNPH1 levels. Silencing HNRNPH1 resulted in an increase in PD-L1 and JAK2 mRNA levels in both A375 cells (FIGS. 5A-B) and U251 cells treated with IFNγ (FIGS. 15A-B). This was associated with a significant increase in PD-L1 and JAK2 protein in both cell lines in response to IFNγ stimulation (FIGS. 5C and 15E). No consistent effect of HNRNPH1 knockdown on INCA1 expression was observed in the two tested cell lines, with INCA1 levels that increased in A375 with both siRNAs but downregulated in U251 transfected with the siHNRNPH1-1 (FIGS. 15C-D). These data suggested that HNRNPH1 is a negative regulator of PD-L1 and JAK2 expression and that binding of INCA1 is required to prevent HNRNPH1 function. This hypothesis was supported by an inverse correlation observed between HNRNPH1 levels and PD-L1 expression in GBM tumors (TCGA) (FIG. 5D). To further prove our hypothesis, we conducted knockdown experiments to silence HNRNPH1 expression in INCA1 knockdown cells. As expected INCA1 knockdown resulted in reduced PD-L1 expression in IFNγ-treated cells. However, silencing HNRNPH1 in those knockdown cells was able to rescue PD-L1 expression in both A375 and U251 cell lines (FIG. 5E and FIG. 15F).

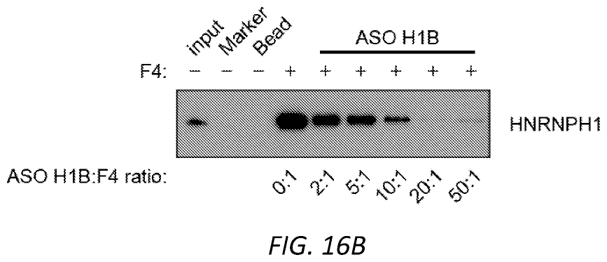

To confirm that the interaction between INCA1 and HNRNPH1 mediates the regulation of PD-L1 and JAK2, we first mapped HNRNPH1 binding sites in vivo by enhanced CLIP sequencing (eCLIP-seq). We identified a cluster of peaks in the proximal intron of INCA1 that were enriched in G-stretches, commonly found in HNRNPH1 interacting RNAs (FIG. 5F). We also identified HNRNPH1 binding sites in the 272 intronic regions of PD-L1 and JAK2, confirming our RIP results (FIG. 5F). To validate sequencing data in vitro, we cloned an INCA1 minigene containing the 5′ and 3′ region of the first intron and used it to generate 7 different in vitro transcribed biotinylated RNA fragments. RNA pull-down assays showed that HNRNPH1 interacted with the RNA fragments containing the sequences of the major peaks found in the eCLIP-seq (FIG. 5G and Table 1). We further proved HNRNPH1 binding to the sequence with strongest eCLIP signal by electrophoretic mobility shift assay (EMSA) and microscale thermophoresis (FIG. 16A and FIG. 5H). Using an antisense oligonucleotide with 2′-O-Methoxyethyl (2′-MOE) modifications targeting the HNRNPH1 binding site (ASO H1B, Table 2) we were able to reduce in vitro INCA1 interaction to HNRNPH1 (FIG. 5I), which occurred in a dose-dependent manner (FIG. 16B). On the contrary, no effect on binding ability was observed using a control ASO (FIG. 5I). To study the effect of ASO H1B in vivo, we transfected A375 melanoma cells with ASO H1B or control ASO. Even though we did not observe changes in INCA1 and PD-L1 RNA levels, ASO H1B significantly reduced INFγ-stimulated PD-L1 protein expression (FIGS. 5J-L). Significant change of JAK2 was observed both at mRNA and protein levels. These results indicate that INCA1 specifically interacts with HNRNPH1 and that blocking this interaction affects PD-L1 and JAK2 expression.

TABLE 1
INCA1 sequences identified to be bound by HNRNPH1.

| | | | | SEQ |
|---|---|---|---|---|
| ID | Chr | Location | Sequence | ID NO: |
| F1 | 9 | 5,629,531.. | CTGGGTGGAAGTTGGGGGTG | 49 |
| | | 5,629,464 | GGACGGCGCCGGGAGGCCGG | |
| | | | GGAGGCAGCGGCGAAAGGCG | |
| | | | GCGGGCGG | |
| F4 | 9 | 5,628,697.. | ATTGGCGGGGTCTCGGTGGG | 50 |
| | | 5,628,629 | GGCGGGCCGGTTGCCGGGGG | |
| | | | GAGCTGGAGTCACGTGACGG | |
| | | | CCGGCGCGG | |
| F5 | 9 | 5,628,607.. | CCGCTGTGGCTGTGGTGGGC | 51 |
| | | 5,628,539 | GGTAGGGGCTTTTACCGTGT | |
| | | | GAGCTCCTTTAATGCCGGGG | |
| | | | TCTGCAGTG | |
| F6 | 9 | 5,628,202.. | TGAATAGAAGGAGGGTTGAG | 52 |
| | | 5,628,134 | GGGATATGGTGTGGAATGGG | |
| | | | GGAAGGAGGAGTCTGAAATA | |
| | | | GAAGAAAGA | |

TABLE 2
Sequence of antisense oligonucleotide
targeting HNRNPH1 binding site.

| | | SEQ |
|---|---|---|
| ID | Sequence | ID NO: |
| ASO H1B | CTCCAGCTCCCCCCGGCAAC* | 8 |

*Modifications: Phosphorothioate Bond/2′-Methoxyethyl

### REFERENCES

1. P. Sharma, J. P. Allison, The future of immune checkpoint therapy. Science 348, 56-61 (2015).
2. S. L. Topalian et al., Safety, activity, and immune correlates of anti-PD-1 antibody in cancer. N Engl J Med 366, 2443-2454 (2012).
3. J. Larkin et al., Combined Nivolumab and Ipilimumab or Monotherapy in Untreated Melanoma. N Engl J Med 373, 23-34 (2015).
4. S. J. Antonia et al., Durvalumab after Chemoradiotherapy in Stage III Non-Small-Cell Lung Cancer. N Engl J Med 377, 1919-1929 (2017).
5. G. P. Dunn, C. M. Koebel, R. D. Schreiber, Interferons, immunity and cancer immunoediting. Nat Rev Immunol 6, 836-848 (2006).
6. A. Garcia-Diaz et al., Interferon Receptor Signaling Pathways Regulating PD-L1 and PD-L2 Expression. Cell Rep 19, 1189-1201 (2017).
7. D. M. Pardoll, The blockade of immune checkpoints in cancer immunotherapy. Nat Rev Cancer 12, 252-264 (2012).
8. D. Pardoll, Cancer and the Immune System: Basic Concepts and Targets for Intervention. Semin Oncol 42, 523-538 (2015).
9. S. L. Topalian, J. M. Taube, R. A. Anders, D. M. Pardoll, Mechanism-driven biomarkers to guide immune checkpoint blockade in cancer therapy. Nat Rev Cancer 16, 275-287 (2016).
10. S. Geisler, J. Coller, RNA in unexpected places: long non-coding RNA functions in diverse cellular contexts. Nat Rev Mol Cell Biol 14, 699-712 (2013).
11. J. R. Prensner et al., Transcriptome sequencing across a prostate cancer cohort identifies PCAT-1, an unannotated lincRNA implicated in disease progression.

Nat Biotechnol 29, 742-749 (2011).
12. T. Gutschner et al., The noncoding RNA MALAT1 is a critical regulator of the metastasis phenotype of lung cancer cells. Cancer Res 73, 1180-1189 (2013).
13. M. Mineo et al., The Long Non-coding RNA HIF1A-AS2 Facilitates the Maintenance of Mesenchymal Glioblastoma Stem-like Cells in Hypoxic Niches. Cell Rep 15, 2500-2509 (2016).
14. J. R. Prensner, A. M. Chinnaiyan, The emergence of lncRNAs in cancer biology. Cancer Discov 1, 391-407 (2011).
15. R. K. Kutty et al., Proinflammatory cytokine interferon-gamma increases the expression of BANCR, a long non-coding RNA, in retinal pigment epithelial cells. Cytokine 104, 147-150 (2018).
16. J. L. Rinn, H. Y. Chang, Genome regulation by long noncoding RNAs. Annu Rev Biochem 81, 145-166 (2012).
17. G. J. Freeman et al., Engagement of the PD-1 immunoinhibitory receptor by a novel B7 family member leads to negative regulation of lymphocyte activation. J Exp Med 192, 1027-1034 (2000).
18. T. Okazaki, S. Chikuma, Y. Iwai, S. Fagarasan, T. Honjo, A rheostat for immune responses: the unique properties of PD-1 and their advantages for clinical application. Nat Immunol 14, 1212-1218 (2013).
19. M. M. D'Aloia, I. G. Zizzari, B. Sacchetti, L. Pierelli, M. Alimandi, CAR-T cells: the long and winding road to solid tumors. Cell Death Dis 9, 282 (2018).
20. A. T. Willingham et al., A strategy for probing the function of noncoding RNAs finds a repressor of NFAT. Science 309, 1570-1573 (2005).
21. M. K. Atianand et al., A Long Noncoding RNA lincRNA-EPS Acts as a Transcriptional Brake to Restrain Inflammation. Cell 165, 1672-1685 (2016).
22. M. Huarte et al., A large intergenic noncoding RNA induced by p53 mediates global gene repression in the p53 response. Cell 142, 409-419 (2010).
23. Y. Hasegawa et al., The matrix protein hnRNP U is required for chromosomal localization of Xist RNA. Dev Cell 19, 469-476 (2010).
24. P. J. Uren et al., High-throughput analyses of hnRNP H1 dissects its multi-functional aspect. RNA Biol 13, 400-411 (2016).
25. M. M. Stevens et al., Drug sensitivity of single cancer cells is predicted by changes in mass accumulation rate. Nat Biotechnol 34, 1161-1167 (2016).
26. C. H. Mermel et al., GISTIC2.0 facilitates sensitive and confident localization of the targets of focal somatic copy-number alteration in human cancers.

Genome Biol 12, R41 (2011).
27. R. Beroukhim et al., The landscape of somatic copy-number alteration across human cancers. Nature 463, 899-905 (2010).
28. A. M. Snijders et al., Assembly of microarrays for genome-wide measurement of DNA copy number. Nat Genet 29, 263-264 (2001).
29. C. W. Brennan et al., The somatic genomic landscape of glioblastoma. Cell 155, 462-477 (2013).
30. T. I. Zack et al., Pan-cancer patterns of somatic copy number alteration. Nat Genet 45, 1134-1140 (2013).
31. L. A. Johnson et al., Rational development and characterization of humanized anti-EGFR variant III chimeric antigen receptor T cells for glioblastoma. Sci Transl Med 7, 275ra222 (2015).
32. K. Shah et al., Bimodal viral vectors and in vivo imaging reveal the fate of human neural stem cells in experimental glioma model. J Neurosci 28, 4406-4413 (2008).
33. C. A. McHugh et al., The Xist lncRNA interacts directly with SHARP to silence transcription through HDAC3. Nature 521, 232-236 (2015).
34. E. L. Van Nostrand et al., Robust transcriptome-wide discovery of RNA-binding protein binding sites with enhanced CLIP (eCLIP). Nat Methods 13, 508-514 (2016).
35. Beatty, G. L., and Gladney, W. L. (2015). Immune escape mechanisms as a guide for cancer immunotherapy. Clin Cancer Res 21, 687-692.
36. Jenkins, R. W., Barbie, D. A., and Flaherty, K. T. (2018). Mechanisms of resistance to immune checkpoint inhibitors. Br J Cancer 118, 9-16.
37. Benci, J. L., Xu, B., Qiu, Y., Wu, T. J., Dada, H., Twyman-Saint Victor, C., Cucolo, L., Lee, D. S. M., Pauken, K. E., Huang, A. C., et al. (2016). Tumor Interferon Signaling Regulates a Multigenic Resistance Program to Immune Checkpoint Blockade. Cell 167, 1540-1554 e1512

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated inhibitory nucleic acid targeting INCA1, wherein the inhibitory nucleic acid comprises a sequence of nucleotides that are identical or complementary to 10 to 50 consecutive nucleotides of SEQ ID NO:1, or an isolated inhibitory nucleic acid targeting HNRNPH1, wherein the inhibitory nucleic acid comprises a sequence of nucleotides that are identical or complementary to 10 to 50 consecutive nucleotides of SEQ ID NO:53.

2. The inhibitory nucleic acid of claim 1, wherein the inhibitory nucleic acid is an antisense oligo (ASO), gapmer, mixmer, shRNA, or siRNA.

3. The inhibitory nucleic acid of claim 1, wherein the inhibitory nucleic acid is modified.

4. The inhibitory nucleic acid of claim 3, wherein the inhibitory nucleic acid comprises one or more modified bonds or bases.

5. The inhibitory nucleic acid of claim 4, wherein the inhibitory nucleic acid comprises one or more peptide nucleic acid (PNA) or locked nucleic acid (LNA) molecules.

6. The inhibitory nucleic acid of claim 4, wherein at least one nucleotide of the inhibitory nucleic acid is a ribonucleic acid analogue comprising a ribose ring having a bridge between its 2′-oxygen and 4′-carbon.

7. The inhibitory nucleic acid of claim 6, wherein the ribonucleic acid analogue comprises a methylene bridge between the 2′-oxygen and the 4′-carbon.

8. The inhibitory nucleic acid of claim 3, wherein at least one nucleotide of the inhibitory nucleic acid comprises a modified sugar moiety selected from a 2′-O-methoxyethyl modified sugar moiety, a 2′-methoxy modified sugar moiety, a 2′-O-alkyl modified sugar moiety, and a bicyclic sugar moiety.

9. The inhibitory nucleic acid of claim 3, wherein the inhibitory nucleic acid comprises at least one modified internucleoside linkage selected from phosphorothioate, phosphorodithioate, alkylphosphonothioate, phosphoramidate, carbamate, carbonate, phosphate triester, acetamidate, carboxymethyl ester, and combinations thereof.

10. The inhibitory nucleic acid of claim 1, wherein the inhibitory nucleic acid is configured such that hybridization of the inhibitory nucleic acid to the INCA1 or HNRNPH1 activates an RNAse H pathway in the cell; and
induces substantial cleavage or degradation of the INCA1 or HNRNPH1 RNA in the cell; or
interferes with interaction of the INCA1 RNA with HNRNPH1 in the cell.

11. A method of treating a subject who has cancer, the method comprising administering to the subject a therapeutically effective amount of the inhibitory nucleic acid of claim 1.

12. The method of claim 11, further comprising administering a therapeutically effective amount of an immunotherapy and/or a chemotherapeutic agent.

13. The method of claim 12, wherein the immunotherapy comprises administration of an immune checkpoint inhibitor and/or chimeric antigen receptor (CAR)-expressing immune effector cells.

14. The method of claim 13, wherein the CAR-expressing immune effector cells are T cells or NK cells.

15. The method of claim 13, wherein the CAR-expressing immune effector cells are autologous to the subject.

16. The method of claim 13, wherein the immune checkpoint inhibitor is an or comprises one or more anti-CD137 antibodies; anti-PD-1 (programmed cell death 1) antibodies; anti-PDL1 (programmed cell death ligand 1) antibodies; anti-PDL2 antibodies; or anti-CTLA-4 antibodies.

17. The method of claim 11, wherein the subject has melanoma, breast, lung, colon, or brain cancer.

18.-24. (canceled)

25. A composition comprising the inhibitory nucleic acid of claim 1, and a pharmaceutically acceptable carrier.
